(19) United States
(12) Patent Application Publication
(10) Pub. No.: US 2025/0345441 A1
(43) Pub. Date: Nov. 13, 2025

(54) ANTICANCER DRUGS AND METHODS OF MAKING AND USING SAME

(71) Applicant: Yale University, New Haven, CT (US)

(72) Inventors: John Deacon, New Haven, CT (US); Venkatareddy Sabbasani, New Haven, CT (US); Denton Hoyer, New Haven, CT (US)

(21) Appl. No.: 19/066,750

(22) Filed: Feb. 28, 2025

Related U.S. Application Data
(63) Continuation of application No. 17/425,629, filed on Jul. 23, 2021, now abandoned.
(60) Provisional application No. 62/796,983, filed on Jan. 25, 2019.

(51) Int. Cl. A61K 47/54; A61K 31/145; A61K 31/196; A61K 31/704

(52) U.S. Cl. CPC A61K 47/555; A61K 31/145; A61K 31/196; A61K 31/704

(57) ABSTRACT

The present invention provides drug modifications for improving biodistribution and/or specificity of an anticancer drug. In certain embodiments, the compound of the invention comprises a drug, a linker and a core acid. The core acid can be varied to tune the properties of the compound within the body such that the compound more selectively distributes to tumors and is, or becomes active in the cytosol.

### CROSS REFERENCE TO RELATED APPLICATIONS

The present application claims priority under 35 U.S.C. § 119(e) to U.S. Provisional Patent Application No. 62/796,983 filed Jan. 25, 2019, which is incorporated herein by reference in its entirety.

### STATEMENT REGARDING FEDERALLY SPONSORED RESEARCH OR DEVELOPMENT

This invention was made with government support under GM073857 awarded by National Institute of Health. The government has certain rights in the invention.

### BACKGROUND OF THE INVENTION

Cancerous solid tumors develop a uniquely acidic microenvironment in the body, through a combination of factors, including ischemia and hypoxia due to rapid growth exceeding their blood supply, their overactive and predominantly glycolytic metabolism, the expression of surface carbonic anhydrases, and the Warburg effect. Although healthy tissues have a slightly basic pH of 7.4, tumors commonly produce a bulk acidic extracellular pH below pH 7, and the pH at cell surfaces is even lower than the bulk extracellular pH values, leading to microenvironments as low as about pH 6. These pH values are known to influence the ionization state of weak ions.

There is a need in the art for chemotherapies that improve on existing drug therapies by exhibiting more selective delivery of chemotherapeutics to tumors in preference to normal tissues. The present invention addresses this need.

### BRIEF SUMMARY OF THE INVENTION

In one aspect, the invention provides a compound of formula (1):

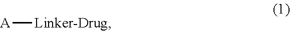

wherein:
A is an acidic group with pK_{A} ranging from about 4.5 to about 7.5,
Linker is a covalent bond or a chemical linker selected such that (1) is selected from the group consisting of:

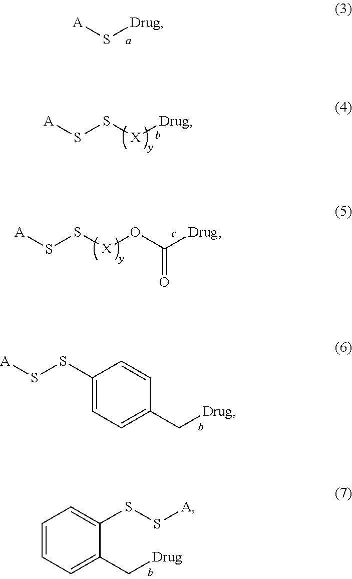

each occurrence of y is independently an integer ranging from 1 to 4;
each occurrence of X is independently selected from the group consisting of CH₂, CH(alkyl) and C(alkyl)₂;
bond a is formed between the sulfur and a substituent on Drug, wherein the substituent is a thiol;
bond b is formed between the carbon and a substituent on Drug, wherein the substituent is selected from the group consisting of hydroxyl, carboxyl, amine, amide, sulfate, sulfonamide, phosphate and phosphoramide;
bond c is formed between the carbonyl and a substituent on Drug, wherein the substituent is selected from the group consisting of primary amine, secondary amine, and hydroxyl; and
Drug is an anticancer drug;
or a salt, solvate, enantiomer, diastereoisomer, geometric isomer or tautomer thereof.

In various embodiments, A is selected from the group consisting of:

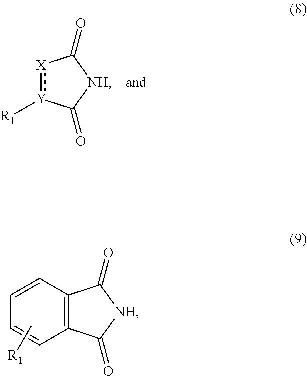

wherein [symbol] is a single or double bond;
wherein each instance of X is independently selected from the group consisting of C, N, S, and O;
wherein each instance of Y is independently selected from the group consisting of C and N; and
wherein R₁ comprises a covalent bond to Linker or Drug.

In various embodiments, A is:

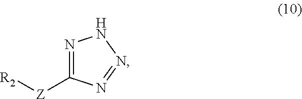

wherein Z is selected from the group consisting of N, C, and aryl, and
wherein R₂ comprises a covalent bond to Linker or Drug.

In various embodiments, A is:

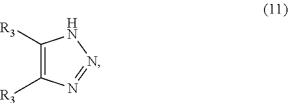

wherein each instance of R₃ is an independently selected electron withdrawing group, or one instance of R₃ is an electron withdrawing group and the other is H, or alkyl; and
wherein at least one instance of R₃ comprises a covalent bond to Linker or Drug either directly or by displacing a hydrogen on an electron withdrawing group, H or alkyl.

In various embodiments, A is:

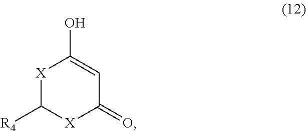

wherein each instance of X is independently selected from the group consisting of C, N, S, and O,
wherein R₄ comprises a covalent bond to Linker or Drug.

In various embodiments, A is:

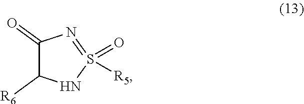

wherein R₆ is selected from the group consisting of an electron withdrawing group, an electron donating group, H, alkyl, and aryl,
wherein R₅ is selected from the group consisting of alkyl and aryl, and
wherein at least one instance of R₆ or R₇ comprises a covalent bond to linker or Drug.

In various embodiments, y is 1 or 2.

In various embodiments, A comprises a carboxylic acid.

In various embodiments, Drug is a pharmaceutically active compound with anticancer, antineoplastic, antimitotic, proapoptotic, antimetastatic, antiangiogenic, cell growth inhibitory, cytostatic, antihormone, immunomodulatory, chemosensitization, and/or radiosensitization activity.

In various embodiments, Drug inhibits topoisomerase II activity.

In various embodiments, the compound is selected from the group consisting of: an anthracycline, an anthraquinone, podophyllotoxin, a quinoline-based compound, naphthalimide, elsamicin A, chartreusin, an acridine, salvicine and derivatives thereof.

In various embodiments, Drug inhibits topoisomerase I activity.

In various embodiments, the compound is selected from the group consisting of: camptothecin, indenoisoquinoline and derivatives thereof.

In various embodiments, the compound is selected from the group consisting of:

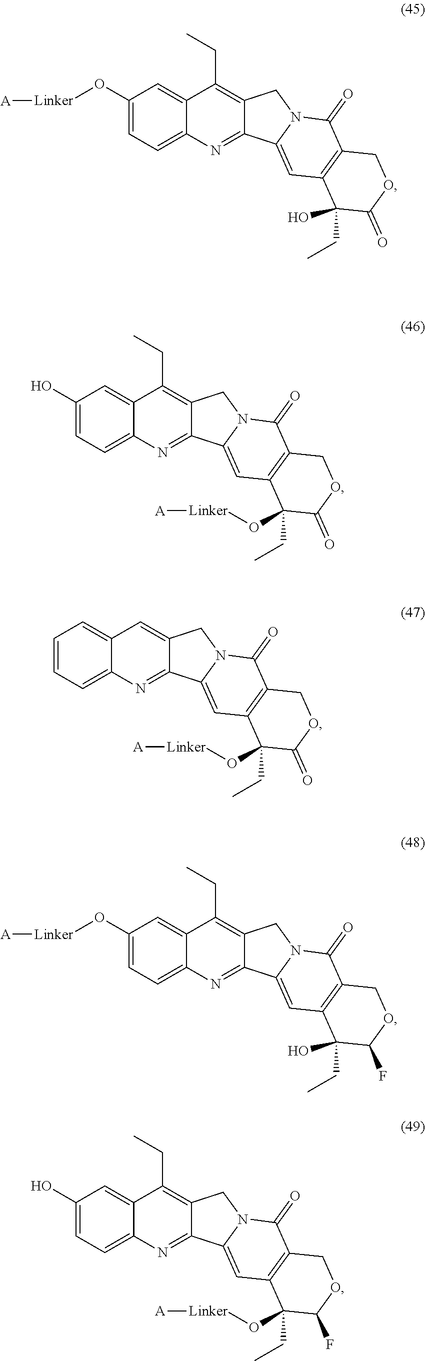

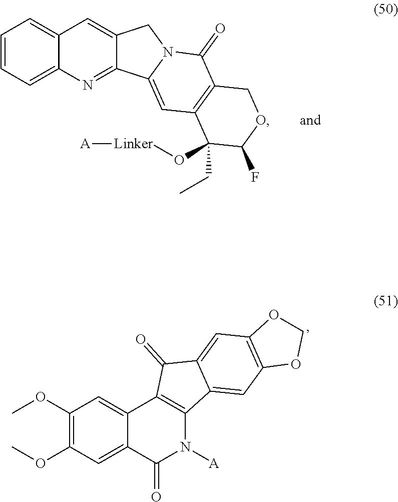

wherein each instance of Linker and A is defined as above.

In various embodiments, Drug inhibits protein kinase activity.

In various embodiments, the compound is an inhibitor of one or more protein kinases selected from the group consisting of: ErbB1, ErbB2, PDGFR, VEGFR, FGFR, ALK, c-Met CDK1, CDK2, CDK4, and CDK6.

In various embodiments, the compound is selected from the group consisting of:

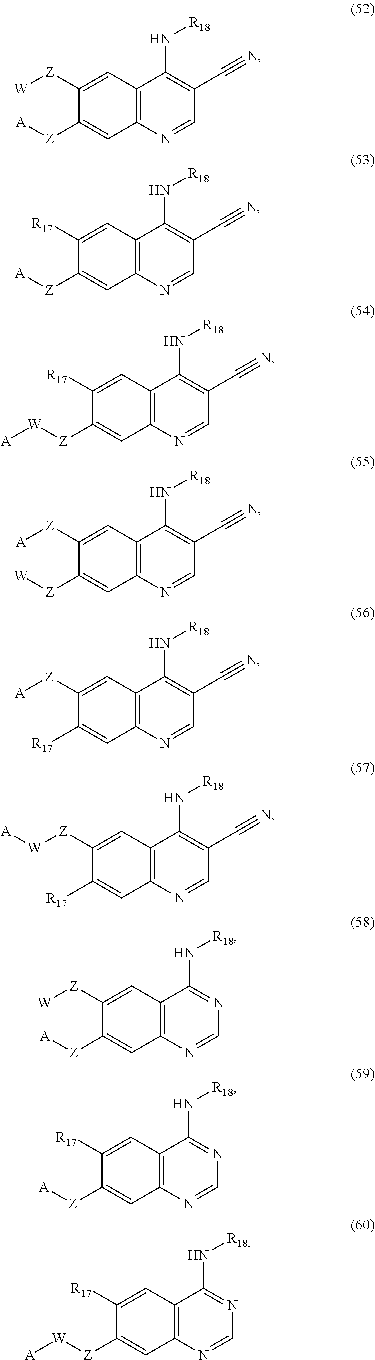

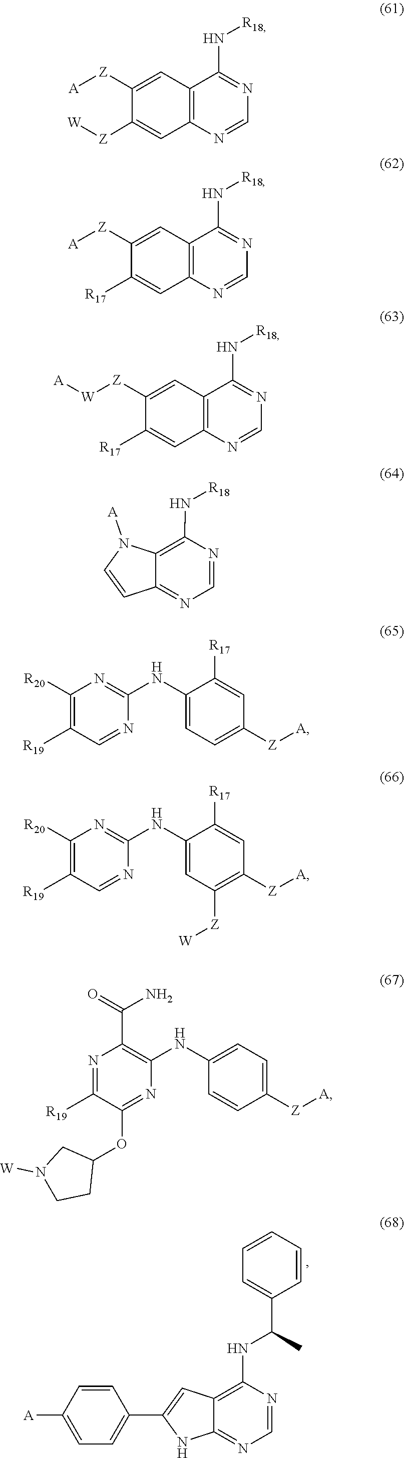

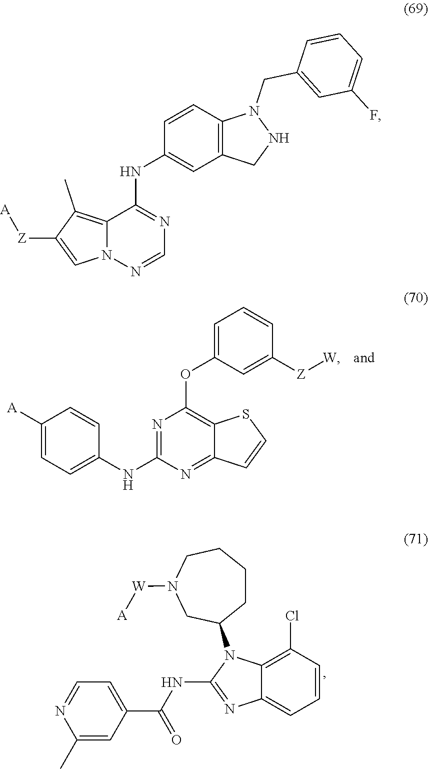

wherein each instance of R₁₇ is independently selected from the group consisting of: H, OH, —O—CH₃, —O—CH₂—CH₃, —O—CH₂—CH₂—O—CH₃, —O—CH₂—CH₂—OH,

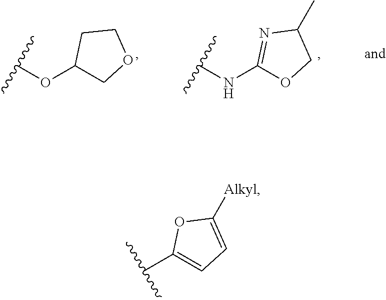

wherein each instance of R₁₈ is independently selected from the group consisting of:

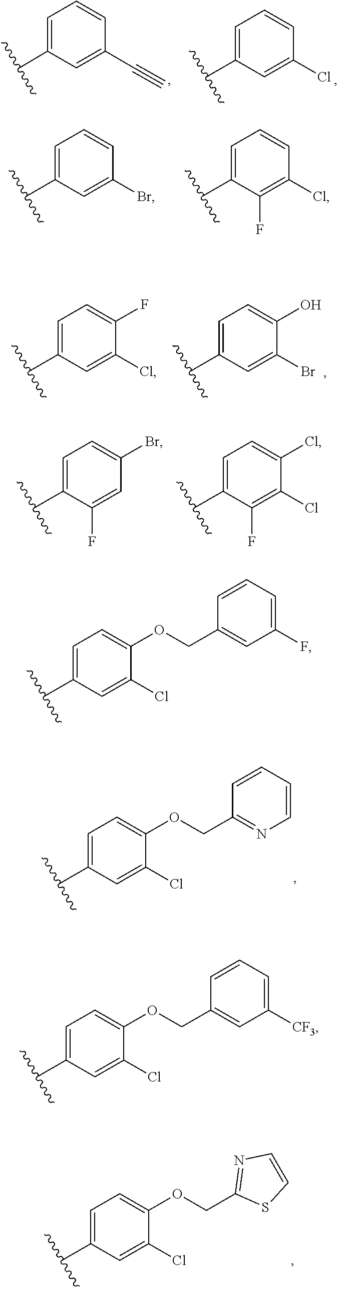

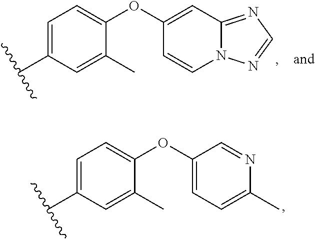 [SMILES: *c1ccc(Oc2ccn3ncnc3c2)c(C)c1 ; *c1ccc(Oc2ccc(C)nc2)c(C)c1]

wherein each instance of R₁₉ is independently selected from the group consisting of: H, F, Cl, Br, I, CF₃, CH₃, ethyl, and alkyl,
wherein each instance of R₂₀ is independently selected from the group consisting of:

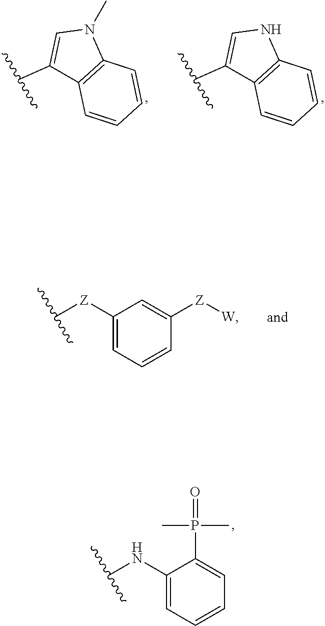

wherein each instance of A is defined as above,
wherein each instance of W is independently selected from the group consisting of:

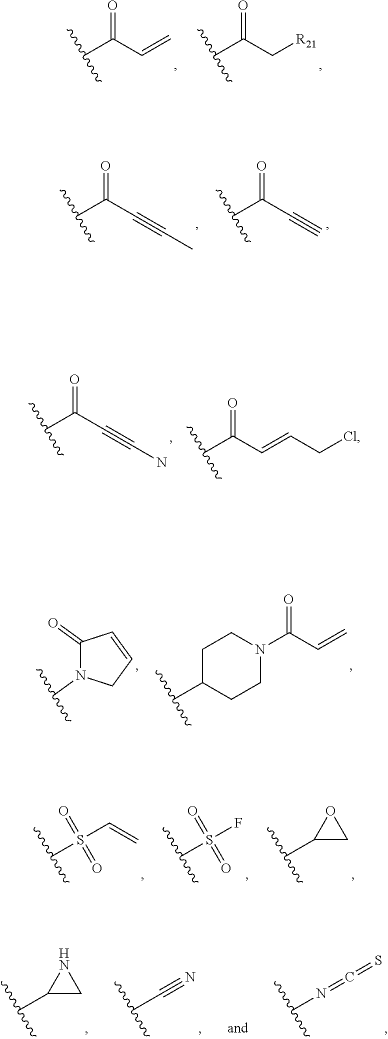

wherein each instance of R₂₁ is independently selected from the group consisting of: F, Cl, Br, I, and N₂;
wherein each instance of Z may be present or absent and where present is independently selected from the group consisting of: O, S, NH, N(methyl), N(alkyl), and CH₂,
wherein the covalent bond between A and W is made in place of a hydrogen on any CH₂ or CH₃ group in W.

In various embodiments, the compound is selected from the group consisting of:

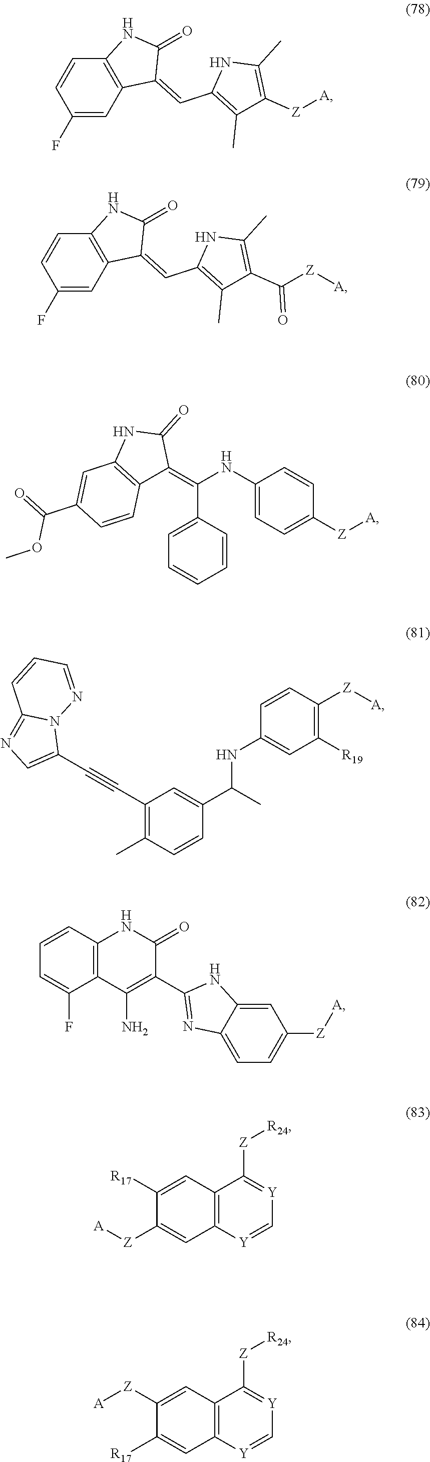

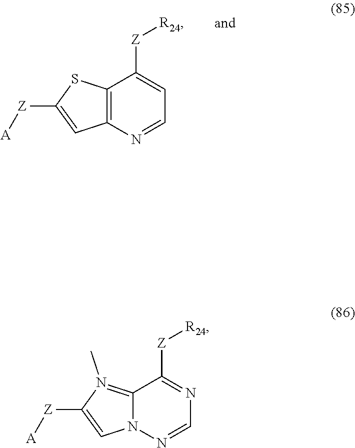

wherein each instance of R₂₄ is independently selected from the group consisting of:

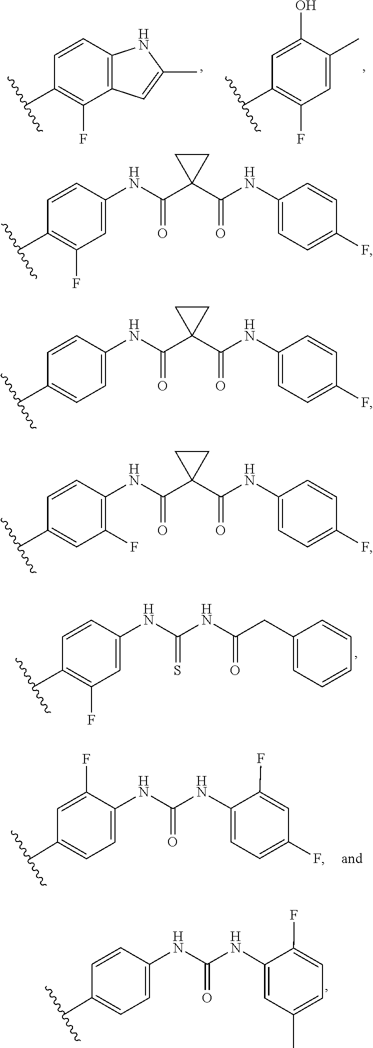

wherein each instance of R₁₇ is independently selected from the group consisting of: H, OH, —O—CH₃, —O—CH₂—CH₃, —O—CH₂—CH₂—O—CH₃, —O—CH₂—CH₂—OH,

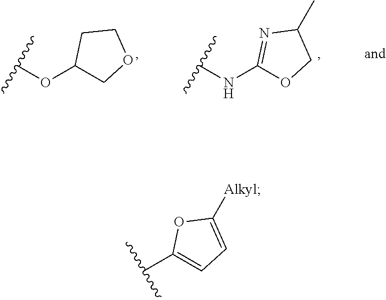

wherein each instance of R₁₉ is independently selected from the group consisting of: H, F, Cl, Br, I, CF₃, CH₃, ethyl, and alkyl,
wherein each instance of Y is independently selected from the group consisting of C and N;
wherein Z may be present or absent and where present is independently selected from the group consisting of O, S, NH, N(methyl), N(alkyl), and CH₂ and
wherein A is defined as above.

In various embodiments, the compound is selected from the group consisting of:

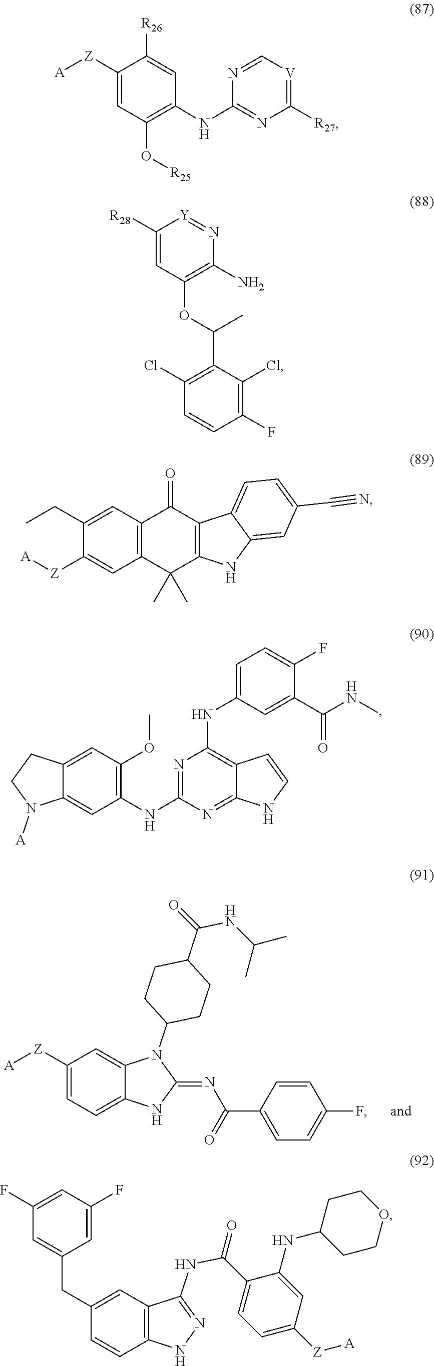

wherein each instance of R₂₅ is independently selected from the group consisting of: methyl and isopropyl,
wherein each instance of R₂₆ is independently selected from the group consisting of: H and methyl,
wherein each instance of R₂₇ is independently selected from the group consisting of:

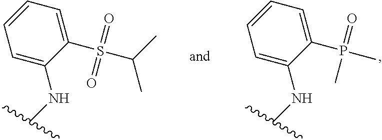 [SMILES: *Nc1ccccc1S(=O)(=O)C(C)C ; *Nc1ccccc1P(C)(C)=O]

wherein each instance of R₂₈ is independently selected from the group consisting of:

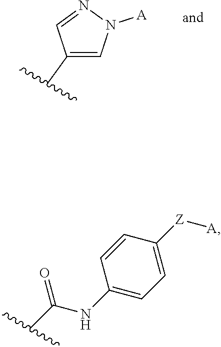

wherein each instance of V is independently selected from the group consisting of: N, CH and CCl;
wherein each instance of Y is independently selected from the group consisting of C and N;
wherein Z may be present or absent and where present is independently selected from the group consisting of: O, S, NH, N(methyl), N(alkyl), and CH₂ and
wherein A is defined as above.

In various embodiments, Drug has PARP inhibition activity.

In various embodiments, the compound is selected from the group consisting of:

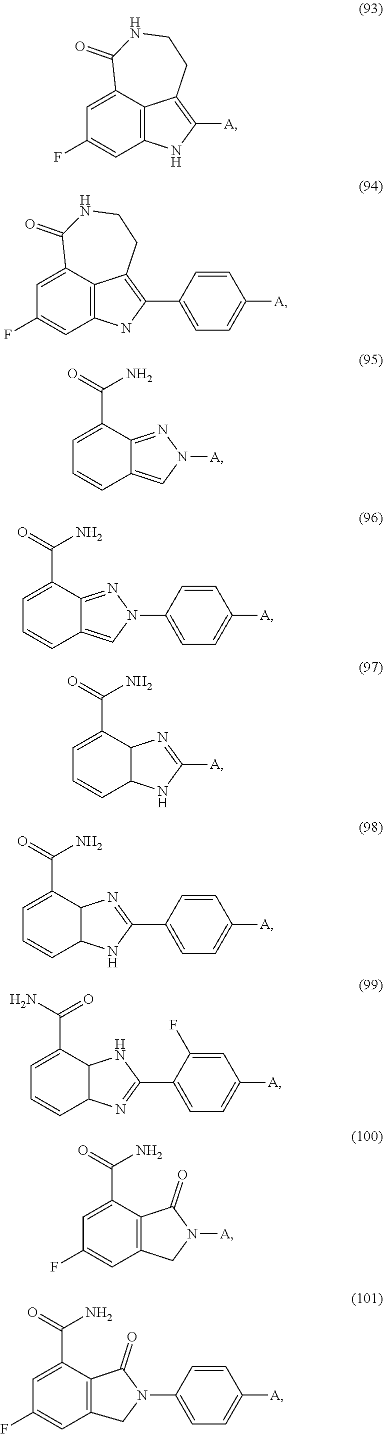

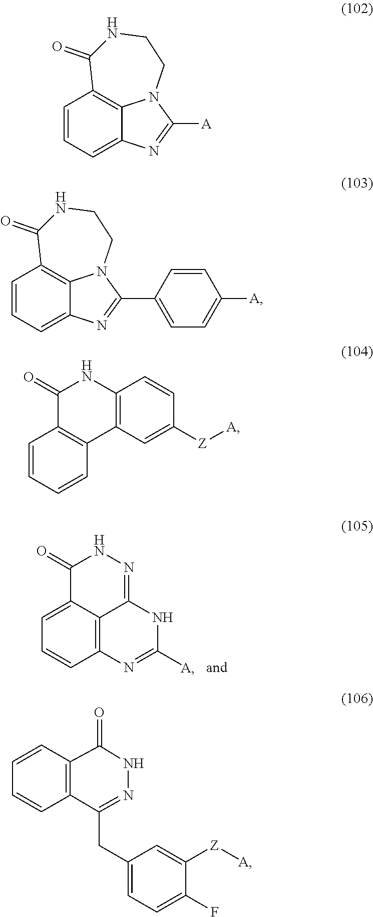

wherein Z may be present or absent and where present is independently selected from the group consisting of: O, S, NH, N(methyl), N(alkyl), and CH₂ and
wherein A is defined as above.

In various embodiments, Drug inhibits estrogen receptor activity.

In various embodiments, the compound is selected from the group consisting of:

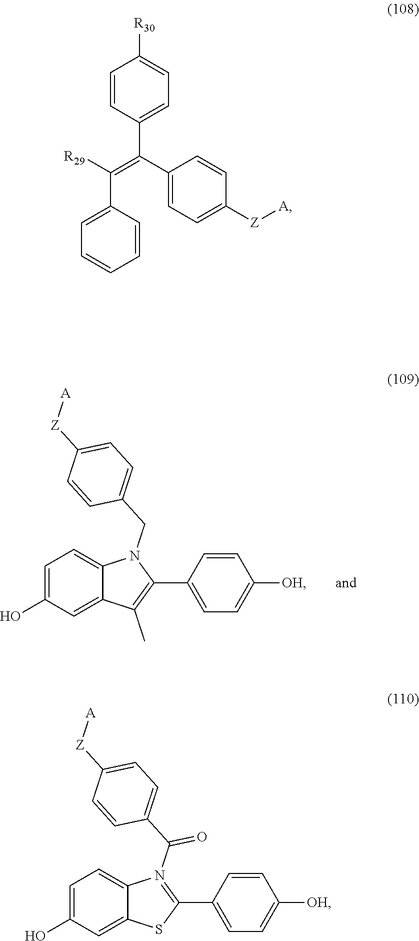

wherein each instance of R₂₉ is independently selected from the group consisting of: ethyl, Cl, and —CH₂—CH₂—Cl, and
wherein each instance of R₃₀ is independently selected from the group consisting of: H and OH
wherein Z may be present or absent and where present is independently selected from the group consisting of: O, S, NH, N(methyl), N(alkyl), and CH₂ and
wherein A is defined as above.

In various embodiments, Drug affects microtubule dynamics.

In various embodiments, the compound is selected from the group consisting of:

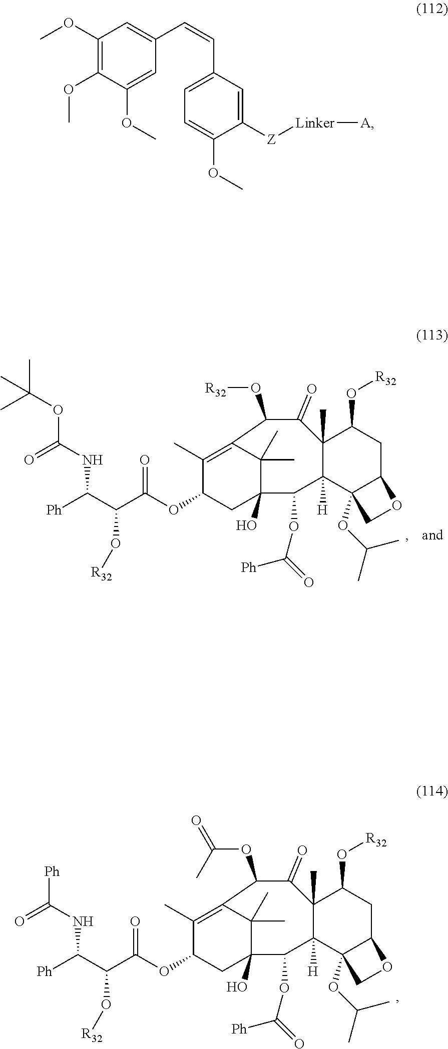

wherein each instance of Linker and A is as defined above,
wherein Z may be present or absent and where present is independently selected from the group consisting of: O, S, NH, N(methyl), N(alkyl), and
wherein each instance of R₃₂, is independently selected from -Linker-A and H, provided that at least one instance of R₃₂ is -Linker-A.

In various embodiments, Drug is a DNA-damaging agent.

In various embodiments, the compound is selected from the group consisting of:

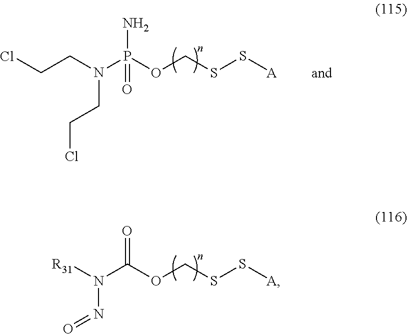

wherein each instance of n is an integer from 1 to 4,
wherein R₃₁ is selected from the group consisting of: methyl, alkyl, and —CH₂—CH₂—Cl, and
wherein each instance of A is defined as above.

In various embodiments, the invention provides a pharmaceutical composition comprising a compound of the invention and a pharmaceutically acceptable carrier.

In various embodiments, the pharmaceutical composition, further comprises at least one additional chemotherapeutic drug.

In various embodiments, the pharmaceutical composition is formulated for nasal, inhalational, topical, oral, buccal, rectal, pleural, peritoneal, vaginal, intramuscular, subcutaneous, transdermal, epidural, intratracheal, otic, intraocular, intrathecal or intravenous administration.

In various embodiments, the invention provides a method for treating a cancer in a subject in need thereof, the method comprising administering to the subject a therapeutically effective amount of a compound or pharmaceutical composition of the invention.

In various embodiments, the compound accumulates in a tumor cell to a greater degree than in a healthy cell in the body.

In various embodiments, the ratio of compound accumulation in the tumor cell with respect to the healthy cell is higher than for Drug alone.

In various embodiments, the cancer is at least one selected from the group consisting of melanoma, breast cancer, prostate cancer, ovarian cancer, uterine cancer, cervical cancer, skin cancer, pancreatic cancer, colorectal cancer, renal cancer, childhood solid tumors, soft-tissue sarcoma, non-hodgkins lymphoma, hepatocellular carcinoma, bladder cancer, testicular cancer, oropharyngeal cancer, head and neck cancer, and lung cancer.

In various embodiments, the invention method of the invention further comprises procuring the compound of or pharmaceutical composition of the invention for the subject.

In various embodiments, the method of the invention further comprising administering to the subject additional cancer treatment.

In various embodiments, the additional cancer treatment is selected from the group consisting of radiation, surgical excision, immunotherapy, and antiproliferative chemotherapy.

In various embodiments, the invention provides a prepackaged pharmaceutical composition comprising a compound or pharmaceutical composition of the invention and an instructional material for use thereof, wherein the instructional material comprises instructions for preventing or treating cancer in a subject.

### BRIEF DESCRIPTION OF THE DRAWINGS

The following detailed description of specific embodiments of the invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIG. 1 depicts a graphical summary of the cellular uptake of ionizable compounds in healthy tissues versus solid tumors. Drugs that exert their effect(s) by interacting with intracellular targets must pass through the cell membrane in order to function. The cell membrane is selectively permeable to small molecules, and highly polar or charged molecules are generally not membrane permeable. As shown, weakly ionic small molecules may exhibit pH-dependent membrane permeability, due to the titration of their ionization state in tissues of different pH. Since solid tumors produce acidic extracellular microenvironments, weakly acidic molecules can gain a tumor-specific enhancement of their membrane permeability, while weak bases can by contrast be less permeable in tumors than in the slightly basic environment around healthy tissues.

FIG. 2 depicts generic and non-limiting examples of cytosolic reductive activation/release of Drug from prodrug, and the probable/predicted side products of linker cleavage and drug release for each type of prodrug linker.

FIGS. 3A-3C depict area under the curve chromatographic data of prodrug and active agent, showing the in vitro kinetics of serum binding and bioavailability in pooled human serum (FIG. 3A), as well as disulfide reduction (FIG. 3B) and drug release in cytosolic conditions (FIG. 3C) of a set of 2-disulfanylethyl carbamate-linked prodrugs of doxorubicin.

FIG. 4A depicts liquid chromatography data showing the stability of YU241528 in serum. While there is an interaction with serum (t_{1/2}>6 hrs), the prodrug is sufficiently stable for its predicted pharmacokinetics, and no active doxorubicin is released.

FIG. 4B depicts representative data showing the release of the active drug, doxorubicin, from YU241528 in conditions simulating the reducing environment in the cytosol. At intracellular reducing conditions (5 mM GSH), doxorubicin is released from the prodrug with a t_{1/2} of ˜2 to 3 hours.

FIGS. 5A-5B depict representative flow cytometry traces of pH-dependent cell treatments. Cultured HeLa cells, treated in suspension for 1 hour at pH 7.4 (X traces) or pH 6.5 (Y traces) with 5 μM doxorubicin or YU241528 prodrug, were washed twice in PBS, pH 7.4, then analyzed by flow cytometry (untreated control cells marked with Z). Normalized cell counts (Y-axes) are plotted vs. doxorubicin fluorescence intensity per cell (X-axes). Doxorubicin exhibits the reported bias of greater uptake into cells at normal physiologic pH 7.4 than into cells at cancerous pH 6.5 (FIG. 5A). YU241528 is preferentially taken up by cells at cancerous pH 6.5 versus cells at normal pH 7.4 (FIG. 5B).

FIG. 5C depicts bar graphs showing the fold bias of doxorubicin and YU241528 towards their respective preferential pH conditions, quantified by flow cytometry above.

FIG. 6 depicts pH-dependent cell growth inhibition of MDA-MB-231 breast cancer cells in culture, treated transiently with Drug (in this case doxorubicin HCl) or various prodrugs of the invention. Cells treated for 6 hours at pH 7.4 (normal physiological pH, X traces) or pH 6.2 (acidic solid tumor pH, Y traces) were subsequently allowed to grow in normal growth medium for 72 hours, then cell viability was evaluated using the CellTiter-Glo assay kit (Promega). Normalized percent cell growth inhibition is plotted on the left side of each panel and IC₅₀ values reported at each pH from non-linear regression analysis. Doxorubicin exhibits the reported bias of greater growth inhibition at basic healthy pH and weakly acidic prodrugs YU244206 and YU241531 exhibit the desired bias of greater growth inhibition at acidic tumor pH, while the non-ionic control prodrug YU245134, which does not significantly change in ionization across the pH range of the assay, exhibits no pH-dependent difference in activity.

FIG. 7 depicts representative in vivo study on the efficacy and toxicity of compounds of the invention. YU241531 (C traces) and YU244206 (B traces) produce similar tumor growth inhibition to doxorubicin (A traces) at its maximum tolerable dose on a once daily for 5 days IV treatment schedule in Balb/c mice with EMT-6 flank tumors, while causing no detectable weight loss compared to sham treated controls (D and E traces). Mean tumor volume and mean body weight graphs are shown for groups of 10 tumor-bearing mice. These data support the core acids' ability to impart selective activity in solid tumors and thus improve upon the parent drug's therapeutic index.

FIG. 8 depicts the dose dependence of YU241531 (bottom panel) and YU244206 (top panel) treatments in the EMT-6 tumor model in Balb/c mice. Mean tumor volume graphs for treatments at 33 mg/kg (B traces) and 100 mg/kg (C traces), IV once daily for 5 days are shown along with sham untreated (D traces) and doxorubicin treated mice (A traces).

FIG. 9 depicts pH-dependent cell growth inhibition of PEO1 ovarian cancer cells in culture, treated transiently with various prodrugs of the invention, as described above. These prodrug exhibit about 3.5 to 10-fold lower IC₅₀ values at tumor pH 6.2 (B traces) than at healthy pH 7.4 (A traces).

FIG. 10 depicts pH-dependent cell growth inhibition of PEO1 ovarian cancer cells in culture, treated transiently with an anticancer kinase inhibitor drug, Osimertinib, or a compound of the invention, based on an active core of that drug, YU253673. While as a weak base, Osimertinib has a slightly lower IC₅₀ value at healthy pH 7.4 (A traces) than at tumor pH 6.2 (B traces), the weakly acidic compound of the invention, YU253673, has far greater activity at tumor pH 6.2, with no observed activity at healthy pH 7.4.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

The term “abnormal”, when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

“About” as used herein when referring to a measurable value such as an amount, a temporal duration, and the like, is meant to encompass variations of 20% or +10%, more preferably +5%, even more preferably 10%, and still more preferably +0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

The term “cancer” refers to the physiological condition in a subject typically characterized by unregulated cell growth. Examples of cancer include, but are not limited to, carcinoma, blastoma, and sarcoma. More particular examples of such cancers include squamous cell cancer (e.g., epithelial squamous cell cancer), melanoma, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, gastrointestinal stromal tumors, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, testicular cancer, hepatic carcinoma, anal carcinoma, penile carcinoma, mouth and throat cancer as well as head and neck cancer.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “core acid” as used herein refers to a small molecule group that can be covalently bonded to a drug or therapeutic molecule, directly or through a linker that can be cleaved inside cells, such as but not limited to, through disulfide reduction in the cancer cell cytosol, thus releasing the drug or therapeutic molecule. In other embodiments, the core acid is not cleaved and remains covalently bonded to the drug or therapeutic molecule. In certain embodiments, the core acid has a pK_{A} between about 4.5 and 7.5 wherein lower pK_{A} values are thought to be more restrictive of drug uptake and to impart more tumor-specific treatment, and wherein higher values are thought to be more permissive of drug uptake and to impart more dose-intensive treatment.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “YU241528” refers to the compound having the structure:

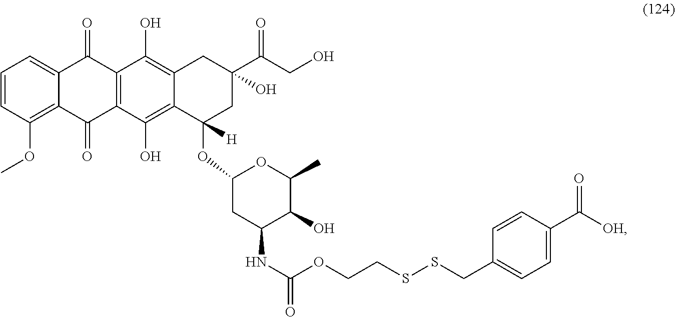 [SMILES: COc1cccc2c1C(=O)c1c(O)c3c(c(O)c1C2=O)C[C@@](O)(C(=O)CO)C[C@@H]3O[C@H]1C[C@H](NC(=O)OCCSSCc2ccc(C(=O)O)cc2)[C@H](O)[C@H](C)O1]

or a salt or solvate thereof.

The term “YU241531” refers to the compound having the structure:

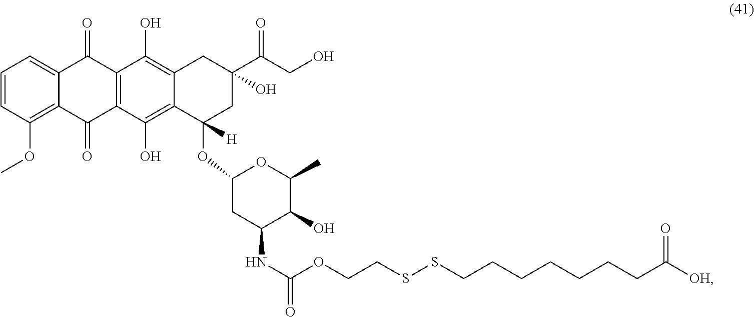 [SMILES: COc1cccc2c1C(=O)c1c(O)c3c(c(O)c1C2=O)C[C@@](O)(C(=O)CO)C[C@@H]3O[C@H]1C[C@H](NC(=O)OCCSSCCCCCCCC(=O)O)[C@H](O)[C@H](C)O1]

or a salt or solvate thereof.

“YU253671” refers to a weakly-acidic prodrug of a mono-chloroethylating agent, having the structure:

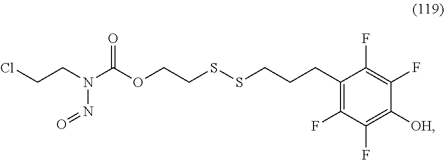 [SMILES: O=NN(CCCl)C(=O)OCCSSCCCc1c(F)c(F)c(O)c(F)c1F]

or a salt or solvate thereof.

“YU252213” refers to a weakly-acidic prodrug of a phosphoramide mustard, having the structure:

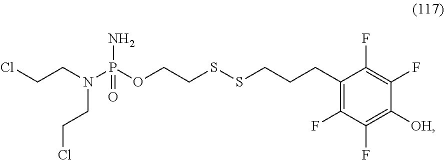 [SMILES: NP(=O)(OCCSSCCCc1c(F)c(F)c(O)c(F)c1F)N(CCCl)CCCl]

or a salt or solvate thereof.

“YU253638” refers to a weakly-acidic prodrug of a mono-methylating agent, having the structure:

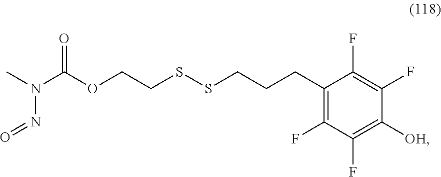 [SMILES: CN(N=O)C(=O)OCCSSCCCc1c(F)c(F)c(O)c(F)c1F]

or a salt or solvate thereof.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

An “electron withdrawing group” as used herein refers to an atom or group of covalently bonded atoms that draws electron density from neighboring atoms towards itself. In certain embodiments, electron withdrawing groups include, but are not limited to, halo, halomethyl, polyhalomethyl, haloalkyl, polyhaloalkyl, aryl, haloaryl, polyhaloaryl, phenyl, benzyl, O-phenyl, cyano, ketone, aldehyde, amido, ester, hydroxy, methoxy, ether, alkene, alkyne, thio, thioether, thioester, nitro, nitroso, sulfonamido (—NH—SO₂-alkyl, —NH—SO₂-aryl, or —SO₂—NH—R where R can be H, alkyl, or aryl) and/or sulfonate (—O—SO₂—R, —SO₂—O—R, or —SO₂—R where R can be alkyl or aryl but not H).

An “electron donating group” as used herein refers to an atom or group that adds electron density to neighboring atoms from itself. In certain embodiments, electron donating groups include, but are not limited to, H, alkyl, cycloalkyl, amino, N-alkyl, N-aryl, O-alkyl, and/or O-aryl.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “procure” or “procuring” as relating to a subject in need of being administered a therapeutically active compound refers to the act of synthesizing, packaging, prescribing, purchasing, providing or otherwise acquiring the compound so that the subject may be administered the compound.

The term “prodrug” refers to a derivatized form of a drug molecule that, while in certain embodiments not pharmacologically active itself, is chemically or enzymatically altered in the body to produce one or more active forms of the drug. A prodrug may in other embodiments be pharmacologically active, but be chemically or enzymatically altered in the body to produce a more active form or a form with different pharmacological activity.

As used herein, the term “small molecule” refers to a molecule of molecular weight equal to or lower than 800 Da, in some embodiments equal to or lower than 700 Da, in some embodiments equal to or lower than 600 Da, in some embodiments equal to or lower than 500 Da, in some embodiments equal to or lower than 400 Da, in some embodiments equal to or lower than 300 Da, in some embodiments equal to or lower than 200 Da, in some embodiments equal to or lower than 100 Da.

A “therapeutic” treatment is a treatment administered to a subject who exhibits signs of pathology, for the purpose of diminishing or eliminating those signs.

The phrase “therapeutically effective amount,” as used herein, refers to an amount that is sufficient or effective to prevent or treat (delay or prevent the onset of, prevent the progression of, inhibit, decrease or reverse) a disease or condition associated with cancer, including alleviating symptoms of such diseases.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

As used herein, “therapeutic index” refers to the ratio of the toxic dose, or dose of a drug that causes adverse effects incompatible with effective treatment of the disease or condition, to the effective dose, or dose of a drug that leads to the desired therapeutic effect in treatment of the disease or condition.

As used herein, “Tumor Activated Permeability” therapy or “TAP” therapy refers to a compound comprising an anticancer drug, a core acid and linker, wherein the linker covalently connects the chemotherapeutic drug and the core acid. The term “TAP group” herein refers to the core acid portion of such a compound.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

In the acidic extracellular environment of a tumor, weakly basic drugs and weakly acidic drugs are affected differently as compared to normal tissues. At physiological pH 7.4, weakly acidic groups, such as carboxyl groups, are predominantly negatively charged, posing an energetic barrier to their diffusion through the cell membrane. However, in acidic environments, such as those that surround cancer cells, acidic groups can become protonated, making them more membrane permeable. This pH-dependent charge favors the biodistribution of weakly acidic drugs into acidic tumors and reduces their permeation of healthy cells. In other words, weakly acidic drugs, or drugs containing titratable weakly acidic groups, can permeate and exert therapeutic effects on cells in acidic tumors to a greater extent than on cells in healthy tissues. However, amine groups are predominantly protonated and positively charged at acidic tumor pH, but deprotonated and uncharged at physiological pH. As a consequence, for weakly basic drugs such as the anthracyclines, or many drugs containing titratable amine groups, this bias is reversed, with such basic drugs more capable of permeating cells in health tissues than acidic tumors. This may help explain the narrow therapeutic index of anthracycline chemotherapy, because these weak base drugs permeate cells in healthy tissues (such as the heart where anthracyclines cause side effects) more easily than in acidic tumors.

Although tumor-targeted treatment methods targeting cell surface receptors overexpressed in certain tumors, such as antibody-based therapeutics, can offer a significant improvement over traditional drug therapies, the rarity of tumor-specific cell-surface biomarkers that differentiate tumor cells from healthy cells sufficiently to facilitate treatment limits the breadth of indications for which they are useful. As a result, the vast majority of patients must rely on non-targeted chemotherapy, and its associated high burden of side effects and lower rate of therapeutic benefit.

In one aspect, the invention includes compounds having the general formula:

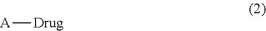

wherein A is a core acid covalently bonded to a Drug. In various embodiments, the core acid is covalently linked to a nitrogen, carbon, oxygen, sulfur, or phosphorus atom within the Drug (wherein a H atom is displaced).

The invention includes a compound comprising a drug that is covalently attached to a core acid through a linker. Non-limiting embodiments of the conjugate are illustrated below, wherein A is the core acid, and Drug is the drug. Formula (1) illustrates a general formula for compounds of the present invention:

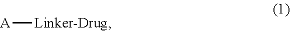

wherein the drug is covalently linked to the Linker through a thiol group within the drug's structure (wherein the H is displaced). The Linker may be a covalent bond, thereby forming compounds as represented by (2).

Linker may be a chemical linker selected such that (1) is selected from the group consisting of:

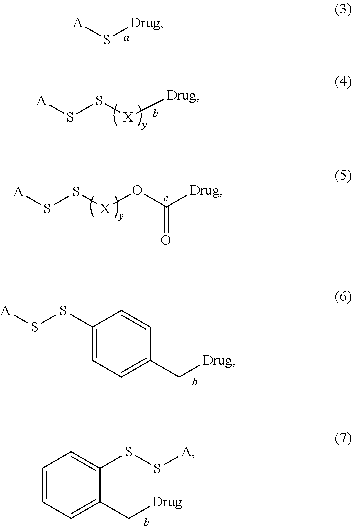

wherein:
each occurrence of y is independently an integer ranging from 1 to 4;
each occurrence of X is independently selected from the group consisting of CH₂, CH(alkyl) and C(alkyl)₂;
bond a is formed between the sulfur and a substituent on Drug, wherein the substituent is a thiol;
bond b is formed between the carbon and a substituent on Drug, wherein the substituent is selected from the group consisting of hydroxyl, carboxyl, amine, amide, sulfate, sulfonamide, phosphate and phosphoramide;
bond c is formed between the carbonyl and a substituent on Drug, wherein the substituent is selected from the group consisting of primary amine, secondary amine, and hydroxyl; and
Drug is an anticancer drug;
or a salt, solvate, enantiomer, diastereoisomer, geometric isomer or tautomer thereof.

In various embodiments, the Linker is a non-cleavable linker selected from the group consisting of alkyl, aryl, polyaromatic, branched alkyl, heteroaryl or polyethylene glycol (PEG).

### Linkers

In certain embodiments, the linker contemplated in the invention forms a covalent bond with a group in the drug (such as an amino, hydroxy and/or thiol group), thus forming a derivative of that group that is not ionized, or where the extent of the group's ionization is reduced, under physiological pH conditions (˜4-9). The covalent bond between the linker and the drug is relatively stable in the bloodstream, but efficiently releases the active drug once inside the targeted cell. In a non-limiting example, to modify an amine group in a drug, a carbamate ethyl disulfide linker can be used. This modification allows for conversion of the amine into a carbamate group, which is two carbons away from a disulfide bond that can be connected to a variety of weakly acidic groups, referred to herein as core acids. Those acidic groups can be tuned to optimize the pH-dependence of the drug's membrane permeability by imparting upon the molecule a predominantly negative charge in pH 7.4 environments and a more neutral charge state in pH 6.2 environments. The disulfide bond acts as a sensor for insertion into the cell, because the concentration of disulfide reducing agents is ˜1,000 times greater in the cytosol of cancer cells than in the blood or interstitial fluid. Upon reduction of the disulfide bond, the freed thiol drives a rearrangement of the linker that forms a thiirane ring and CO₂, releasing the drug with the original amine, now inside the cell.

In certain embodiments, the kinetics of the linker reaction to release the drug have a half-life of between about a few minutes to about a few hours in intracellular reducing conditions. In other embodiments, the kinetics of the linker reaction to release the drug have a half-life of less than a minute in intracellular reducing conditions.

### Core Acids

In certain embodiments, the core acid is a weak acid with an acid dissociation constant (pK_{A}) between about 4.5 and about 7.5 and includes one R group that comprises a covalent bond to the Linker or Drug. In other embodiments, one or more groups with varying electronegative character are appended to the core acid to alter the acid dissociation constant. Electron withdrawing groups can be, but are not limited to, halo, halomethyl, polyhalomethyl, haloalkyl, polyhaloalkyl, aryl, haloaryl, polyhaloaryl, phenyl, benzyl, 0-phenyl, cyano, ketone, aldehyde, amido, ester, hydroxy, methoxy, ether, alkene, alkyne, thio, thioether, thioester, nitro, nitroso, sulfonamido (—NH—SO₂-alkyl, —NH—SO₂-aryl, or —SO₂—NH—R where R can be H, alkyl, or aryl) and/or sulfonate (—O—SO₂—R, —SO₂—O—R, or —SO₂—R where R can be alkyl or aryl but not H), may be present alone or in combinations, including combinations both within the same R-group or combined separately on different R-groups, may be linear, branched or cyclic, and may contact the core acid structure in one or more locations. Electron donating groups can be, but are not limited to, H, alkyl, cycloalkyl, amino, N-alkyl, N-aryl, O-alkyl, and/or O-aryl.

In yet other embodiments, the core acid is selected from the group consisting of:

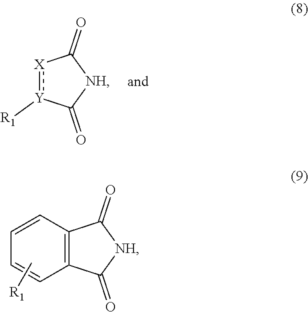

wherein [symbol] is a single or double bond; wherein each instance of X is independently selected from the group consisting of C, N, S, and O; wherein each instance of Y is independently selected from the group consisting of C and N; and wherein R₁ comprises a covalent bond to Linker or Drug.

In various embodiments, A is:

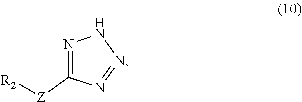

wherein Z is selected from the group consisting of N, C, and aryl, and wherein R₂ comprises a covalent bond to Linker or Drug. In various embodiments, Z is optionally substituted phenyl or optionally substituted naphthyl.

In various embodiments A is:

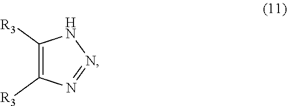

wherein each instance of R₃ is an independently selected electron withdrawing group, or where one instance of R₃ is an electron withdrawing group and the other is an independently selected H, or alkyl group, and wherein at least one instance of R₃ comprises a covalent bond to Linker or Drug either directly or by displacing a hydrogen on an electron withdrawing group, H, or alkyl.

In various embodiments A is:

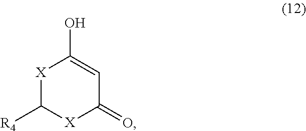

wherein each instance of X is independently selected from the group consisting of C, N, S, and O, wherein R₄ comprises a covalent bond to Linker or Drug.

In various embodiments A is:

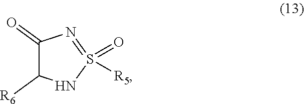

wherein R₆ is selected from the group consisting of an electron withdrawing group, an electron donating group, H, alkyl, and aryl, wherein R₅ is selected from the group consisting of alkyl and aryl, and wherein at least one instance of R₆ or R₇ comprises a covalent bond to linker or Drug.

### Drugs

In certain embodiments, the Drug is a chemotherapeutic drug, which has cytotoxic and/or anticancer activity. In other embodiments, the drug comprises or can be derivatized to comprise a primary amine, secondary amine, a hydroxyl, or a thiol. In yet other embodiments, the drug is a basic or neutral anticancer drug. A person of skill in the art will recognize that the disclosure may be applied to chemotherapeutic drugs of known efficacy, as well as compounds which efficacy has not previously been appreciated.

In various embodiments, the Drug is an anticancer drug. As used herein, the term “anticancer drug” refers to any drug used for its anti-tumor effects including, by way of non-limiting example cytotoxic chemotherapy agents and targeted therapies that interfere with one or more pathways necessary for tumor growth, and/or survival. In various embodiments, the Drug may be the active core or “Effector” covalently linked, either directly or through a linker, to the core acid at a variable group position. A skilled person understands that various drugs contain an active moiety and variable groups, where the active moiety is responsible for exerting the therapeutic effect and the variable groups may be altered to modulate, for example, pharmacokinetic properties of the compound without directly affecting the activity of the active core. In such cases, the term “Drug” as used herein is intended to include both the complete compound with variable groups and the active core as well as the active core alone. By way of non-limiting example, Drug may refer to the active core of a drug and the core acid may replace one or more of the variable groups associated with that active core. Similarly, “anticancer drug” refers to the complete compound or the active core of an anticancer drug.

Without wishing to be limited by theory, a Drug may be considered to be useful as part of a compound of the invention herein if it is a small molecule, exerts antitumor activity via an intracellular target, contains as part of its structure or can be altered into an active derivative or precursor of the active agent that contains as part of its structure one or more variable groups or one or more reactive groups from the list: primary amine, secondary amine, hydroxyl, phosphate, phosphoramide, or thiol, and if the agent in its circulating composition would not contain any strongly ionic groups that would bear a formal charge throughout the range of pH 4 through pH 8, and thereby interfere with the core acid controlling the ionization state of the compound in the body.

The Drug can, but is not limited to, exert its primary antitumor activity through: alkylating activity, by way of non-limiting example, the Drug may be a nitrogen mustard; a cytoskeletal or microtubule disruptor, by way of non-limiting example a taxane; antimetabolic activity, by way of non-limiting example a nucleoside analogue; a drug possessing cytostatic activity, by way of non-limiting example a receptor tyrosine kinase inhibitor; a drug possessing antihormone activity, by way of non-limiting example a selective estrogen receptor modulator; or other mechanisms known in the art to achieve antitumor activity in vivo.

In certain embodiments, the Drug is a basic or neutral chemotherapy drug. In some embodiments, the conjugation of the core acid to the drug improves biodistribution, solubility and/or other developability properties of the drug. In other embodiments, the linker allows for modification of the drug into a prodrug with improved biodistribution, wherein the linker is traceless. In yet other embodiments, the linker converts a basic amine structure to a neutral carbamate structure. In yet other embodiments, a traceless linker covalently connects two chemical species, then releases one or both without any remaining modification to the original structure of the released agent. In yet other embodiments, the linker acts as a sensor for cell insertion, responding to the reductive environment of the cytosolic compartment inside a cell by allowing for traceless release of the drug. In yet other embodiments, use of a linker contemplated within the invention improves biodistribution, solubility and/or other developability properties of the drug. In yet other embodiments, the prodrug is an easier clinical development than the drug itself.

In certain embodiments, a linker contemplated within the invention is used to produce traceless, weakly acidic prodrug modifications of weakly basic drugs. In other embodiments, such modifications improve the therapeutic index and/or therapeutic efficacy of weakly basic drugs, whereby the prodrug enjoys the biodistribution advantage of weakly acidic compounds while in the bloodstream, and then releases the active weakly basic drug inside the cell. In yet other embodiments, the covalent linker is stable in blood, but less stable (more unstable) in the cytosol of a tumor cell and/or undergoes cleavage and/or spontaneously rearrangement in the cytosolic compartment of cells, so as to release the active drug in its original form. In yet other embodiments, the covalent linker is stable outside of cells.

In various embodiments, Drug inhibits topoisomerase I activity. In various embodiments, the compound is selected from the group consisting of: camptothecin, indenoisoquinoline and derivatives thereof. In various embodiments, the compound is selected from the group consisting of:

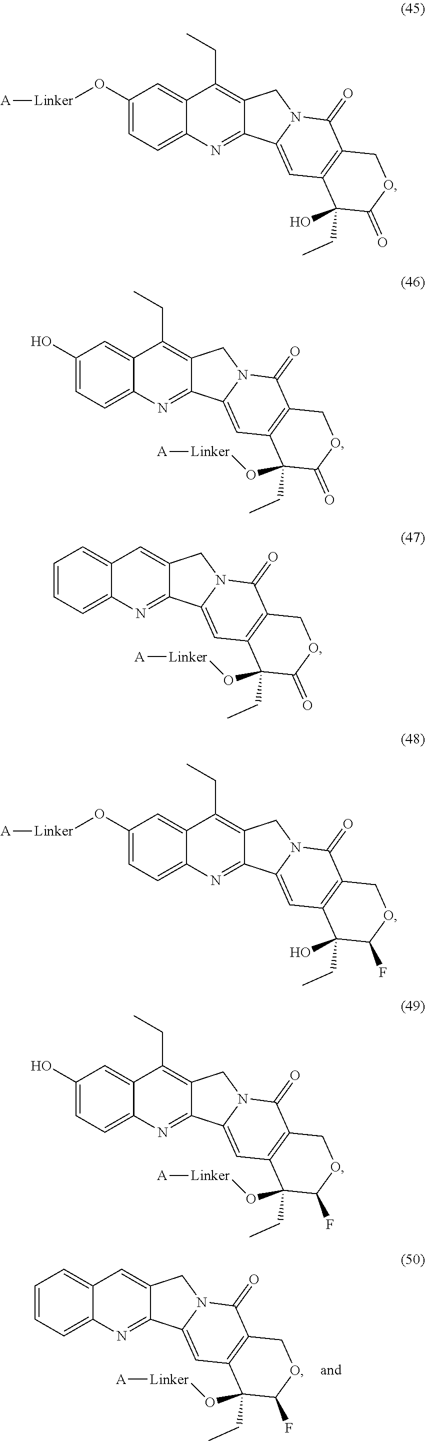

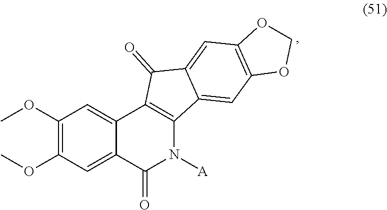

wherein each instance of Linker and A is defined as above.

In various embodiments, Drug inhibits protein kinase activity. In various embodiments, the compound is an inhibitor of one or more protein kinase selected from the group consisting of: ErbB1, ErbB2, PDGFR, VEGFR, FGFR, ALK, c-Met, CDK1, CDK2, CDK4, and CDK6.

In various embodiments, the compound is selected from the group consisting of:

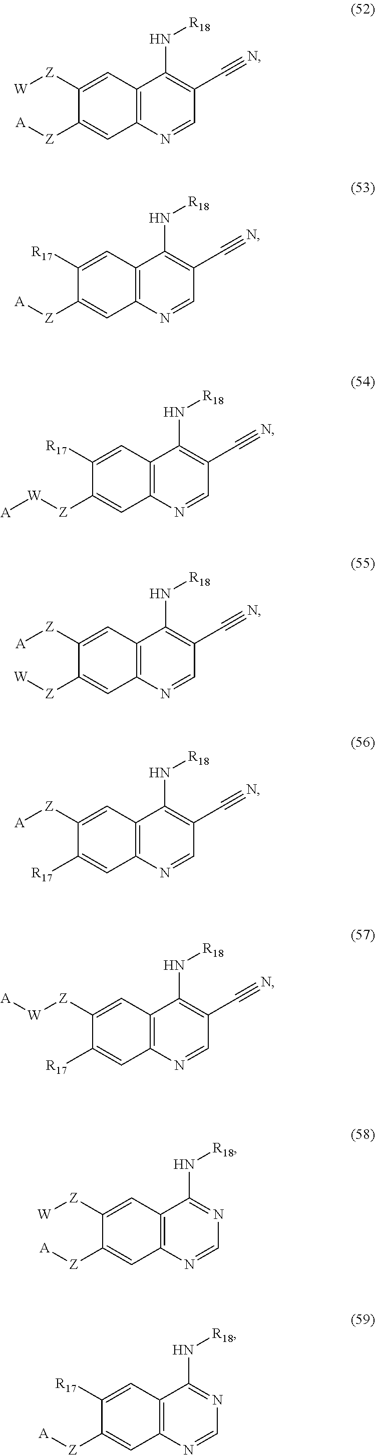

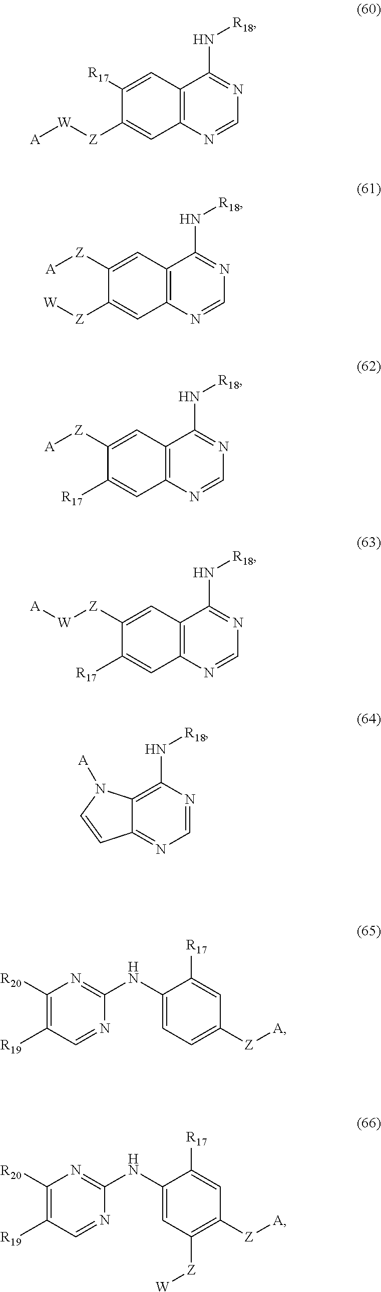

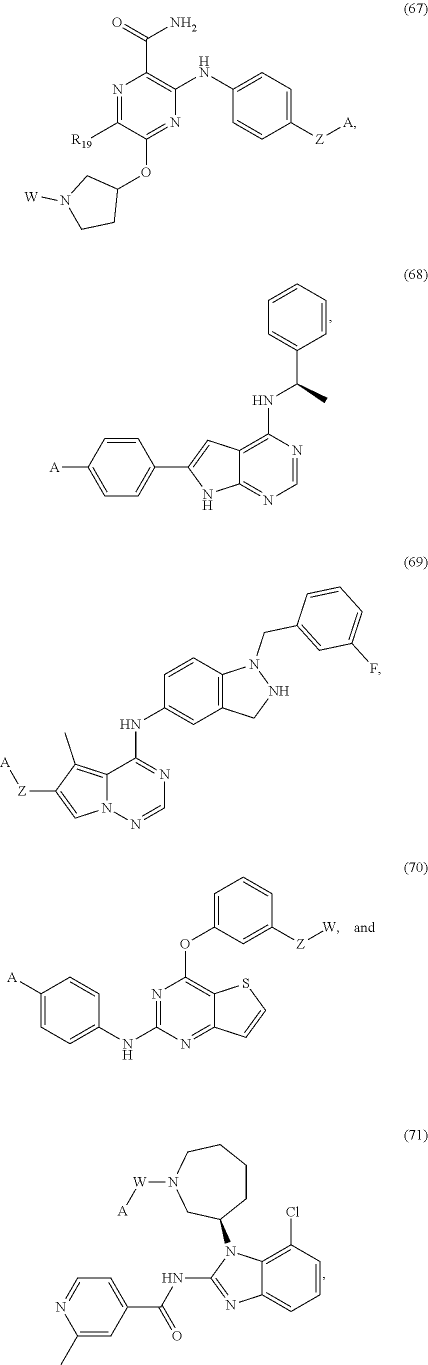

wherein each instance of R₁₇ is independently selected from the group consisting of: H, OH, —O—CH₃, —O—CH₂—CH₃, —O—CH₂—CH₂O—CH₃, —O—CH₂—CH₂—OH,

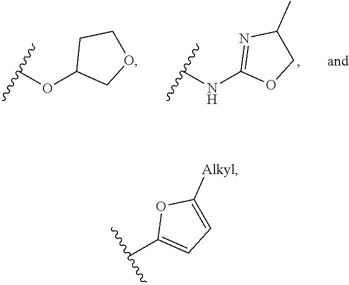

wherein each instance of R₁₈ is independently selected from the group consisting of:

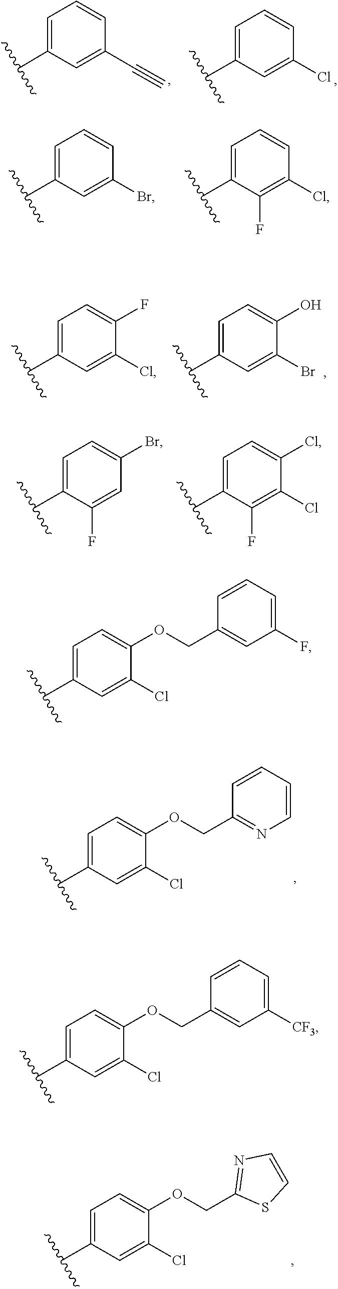

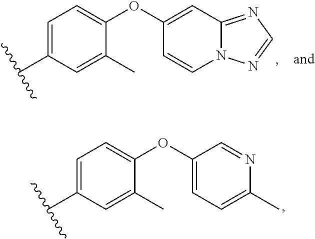 [SMILES: *c1ccc(Oc2ccn3ncnc3c2)c(C)c1 ; *c1ccc(Oc2ccc(C)nc2)c(C)c1]

wherein each instance of R₁₉ is independently selected from the group consisting of: H, F, Cl, Br, I, CF₃, CH₃, ethyl, and alkyl,
wherein each instance of R₂₀ is independently selected from the group consisting of:

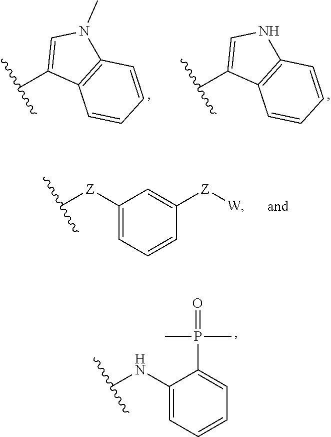

wherein each instance of A is defined as above,
wherein each instance of W is independently selected from the group consisting of:

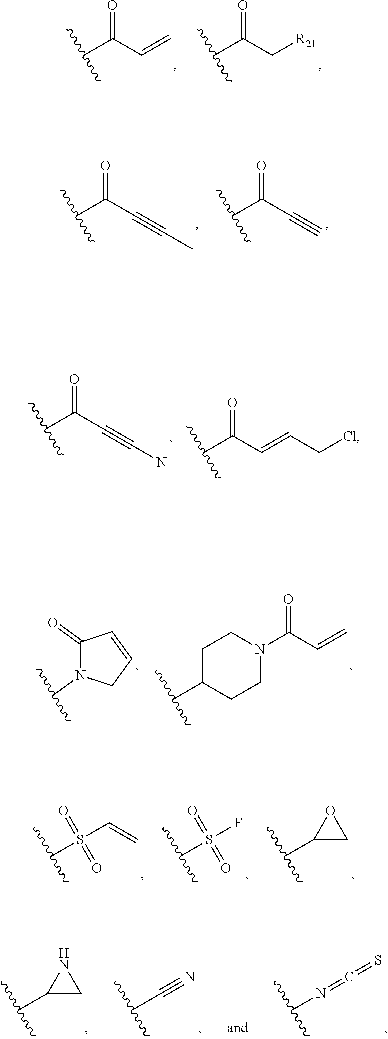

wherein each instance of R₂₁ is independently selected from the group consisting of: F, Cl, Br, I, and N₂;
wherein each instance of Z may be present or absent and where present is independently selected from the group consisting of: O, S, NH, N(methyl), N(alkyl), and CH₂,
wherein the covalent bond between A and W is made in place of a hydrogen on any CH₂ or CH₃ group in W.

In various embodiments, the compound is selected from the group consisting of:

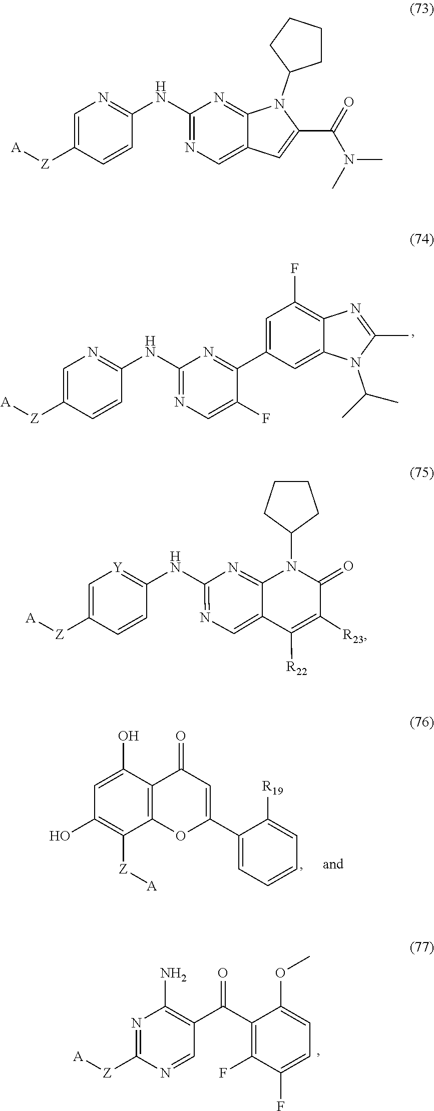

wherein each instance of R₁₉ is independently selected from the group consisting of: H, F, Cl, Br, I, CF₃, CH₃, ethyl, and alkyl,
wherein each instance of R₂₂ is independently selected from H or CH₃,
wherein each instance of R₂₃ is independently selected from acetyl or cyano,
wherein each instance of Y is independently selected from C or N,
wherein Z may be present or absent and where present is independently selected from the group consisting of: O, S, NH, N(methyl), N(alkyl), and CH₂ and
wherein A is defined as above.

In various embodiments, the compound is selected from the group consisting of:

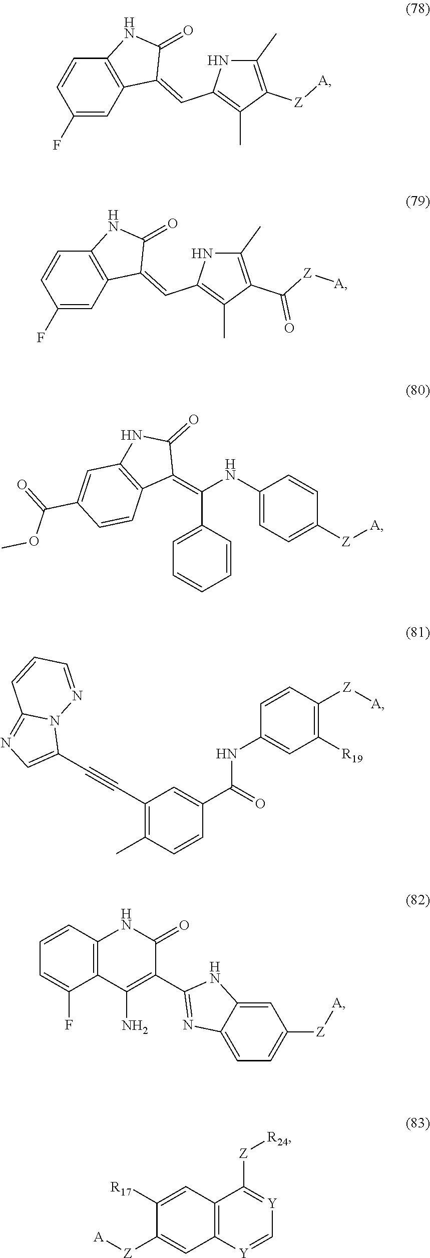

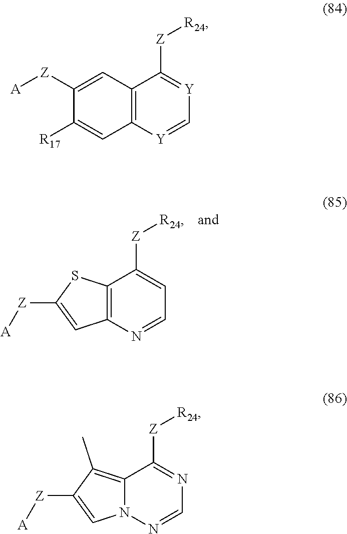

wherein each instance of R₂₄ is independently selected from the group consisting of:

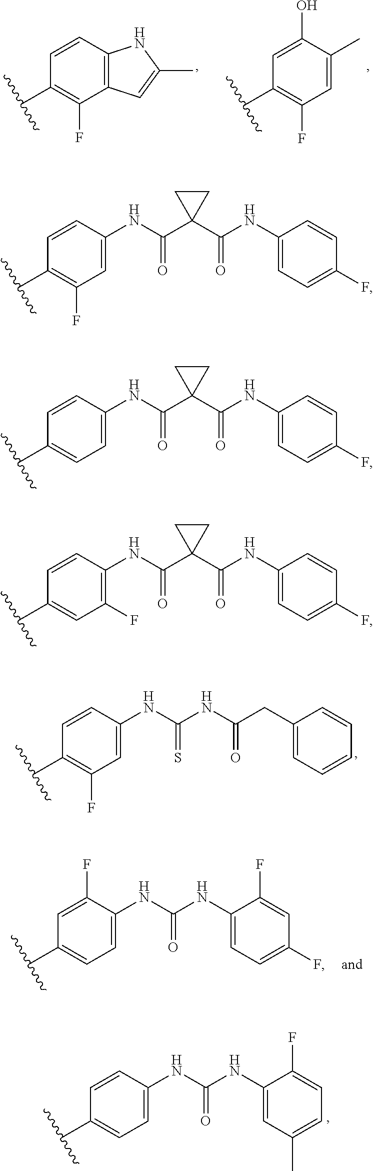

wherein each instance of R₁₇ is independently selected from the group consisting of: H, OH, —O—CH₃, —O—CH₂—CH₃, —O—CH₂—CH₂—O—CH₃, —O—CH₂—CH₂—OH,

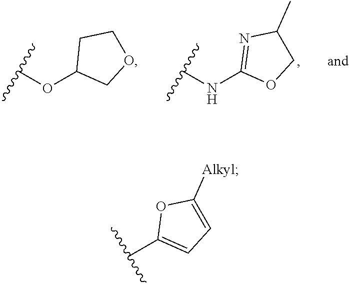

wherein each instance of R₁₉ is independently selected from the group consisting of: H, F, Cl, Br, I, CF₃, CH₃, ethyl, and alkyl,
wherein each instance of Y is independently selected from C or N;
wherein Z may be present or absent and where present is independently selected from the group consisting of: O, S, NH, N(methyl), N(alkyl), and CH₂ and
wherein A is defined as above.

In various embodiments, the compound is selected from the group consisting of:

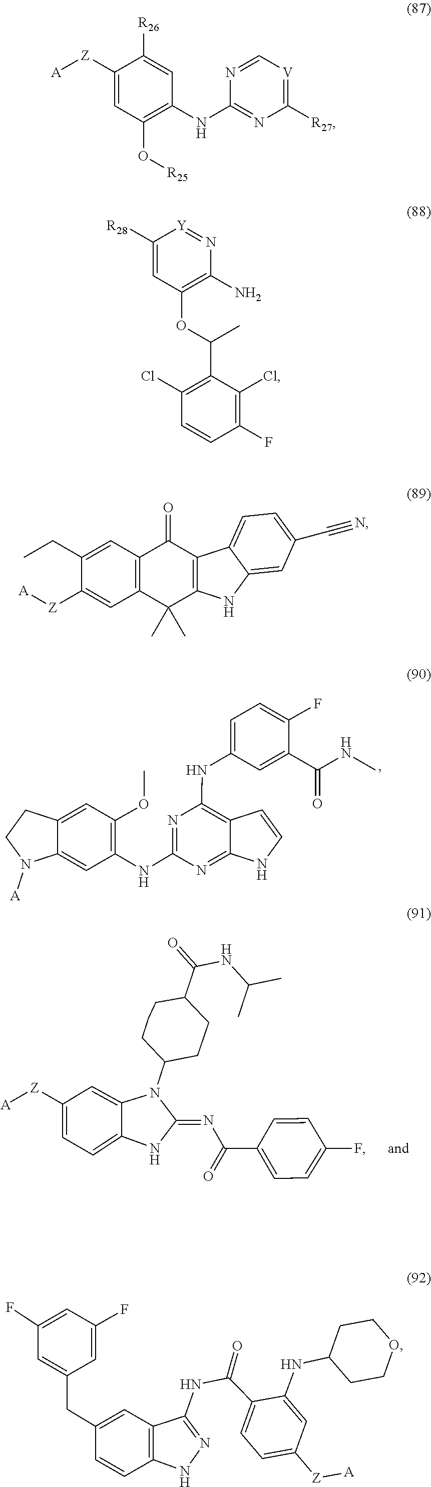

wherein each instance of R₂₅ is independently selected from the group consisting of: methyl and isopropyl,
wherein each instance of R₂₆ is independently selected from the group consisting of: H and methyl,
wherein each instance of R₂₇ is independently selected from the group consisting of:

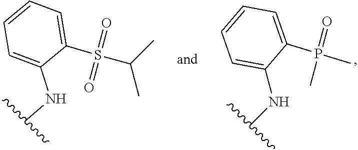

wherein each instance of R₂₈ is independently selected from the group consisting of:

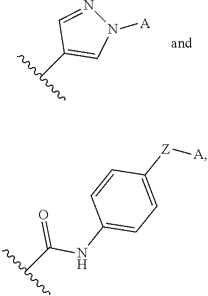

wherein each instance of V is independently selected from the group consisting of: N, CH and CCl;
wherein each instance of Y is independently selected from C or N;
wherein Z may be present or absent and where present is independently selected from the group consisting of: O, S, NH, N(methyl), N(alkyl), and CH₂ and
wherein A is defined as above.

In various embodiments, the Drug has PARP inhibition activity.

In various embodiments, the compound is selected from the group consisting of:

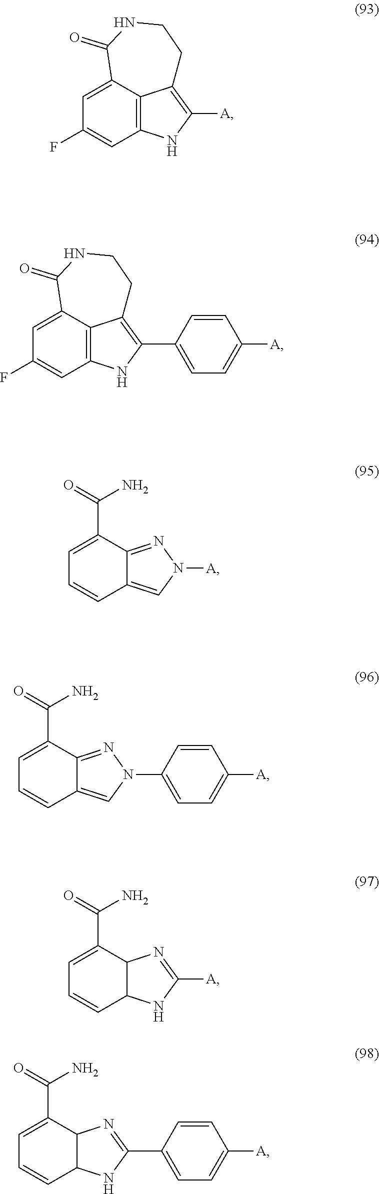

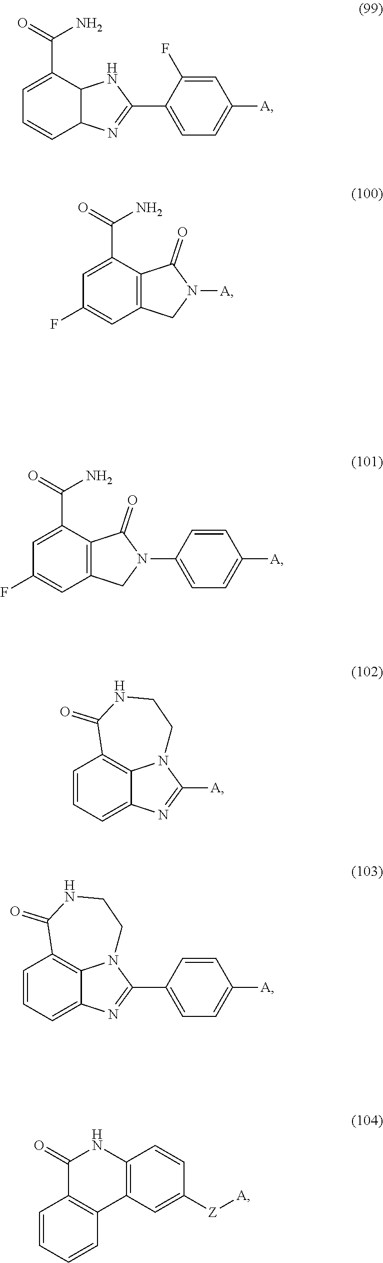

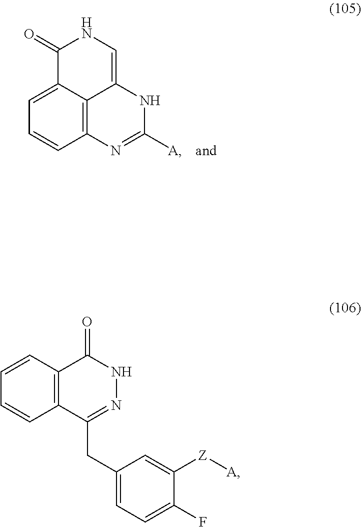

wherein Z may be present or absent and where present is independently selected from the group consisting of: O, S, NH, N(methyl), N(alkyl), and CH₂ and wherein A is defined as above.

In various embodiments, Drug inhibits estrogen receptor activity.

In various embodiments, the compound is selected from the group consisting of:

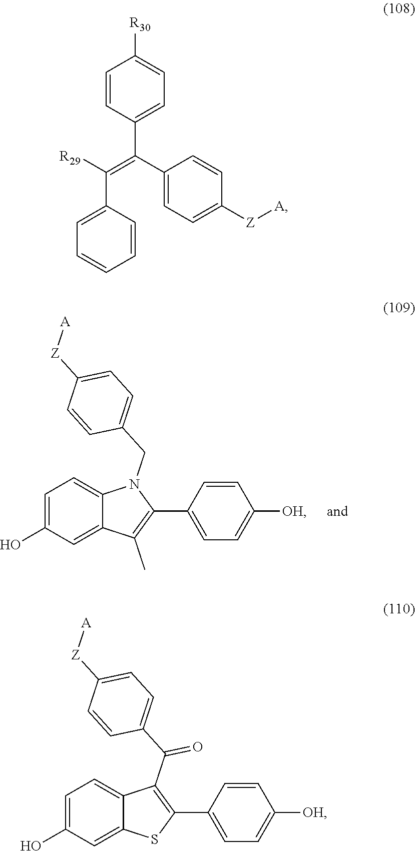

wherein each instance of R₂₉ is independently selected from the group consisting of: ethyl, Cl, and —CH₂—CH₂—Cl, and
wherein each instance of R₃₀ is independently selected from H or OH
wherein Z may be present or absent and where present is independently selected from the group consisting of: O, S, NH, N(methyl), N(alkyl), and CH₂ and
wherein A is defined as above.

In various embodiments, Drug affects microtubule dynamics.

In various embodiments, the compound is selected from the group consisting of:

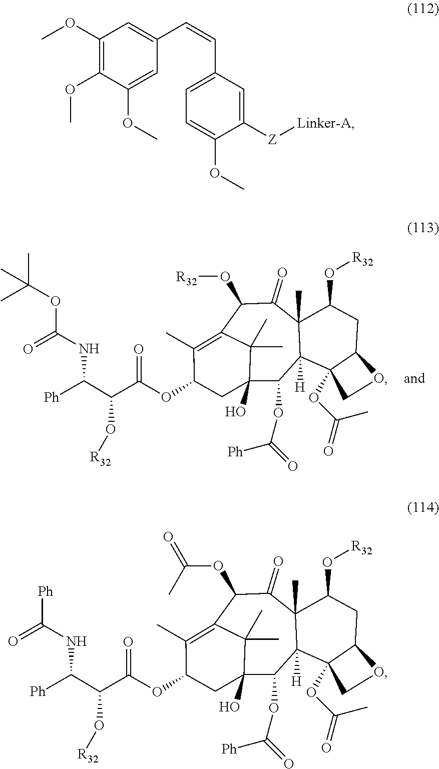

wherein each instance of Linker and A is as defined above,
wherein Z may be present or absent and where present is independently selected from the group consisting of: O, S, NH, N(methyl), N(alkyl), and CH₂, and
wherein each instance of R₃₂, is independently selected from -Linker-A and H, provided that at least one instance of R₃₂ is -Linker-A.

In various embodiments, Drug is a DNA-damaging agent.

In various embodiments, the compound is selected from the group consisting of:

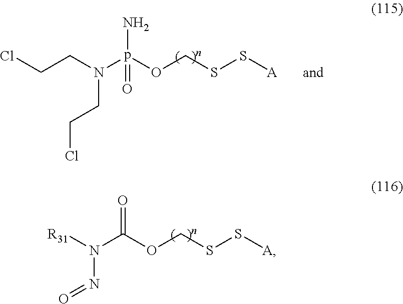

wherein each instance of n is an integer from 1 to 4,
wherein R₃₁ is selected from the group consisting of: methyl, alkyl, and —CH₂—CH₂—Cl, and
wherein each instance of A is defined as above.

The compounds of the invention can possess one or more stereocenters, and each stereocenter may exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol, or buffered solutions thereof. In other embodiments, the compounds described herein exist in unsolvated form.

In certain embodiments, the compounds of the invention may exist as tautomers. All tautomers are included within the scope of the compounds presented herein.

In certain embodiments, compounds described herein are prepared as prodrugs. A “prodrug” refers to an agent that is converted into an active therapeutic compound in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining biodistribution or substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels. In various embodiments, labeled compounds may be used for diagnostic applications wherein the compounds are preferentially absorbed by tumor tissues over healthy tissues and detected using a suitable technique, as appropriate for the label.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

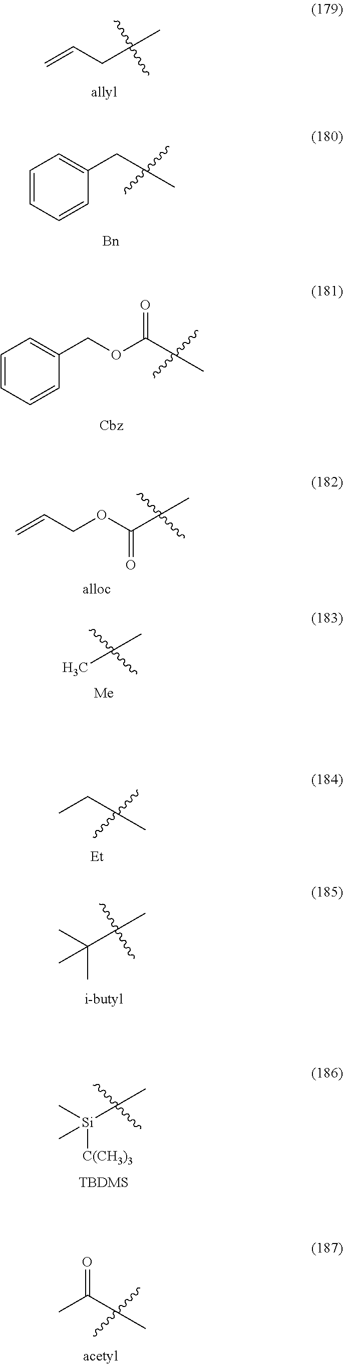

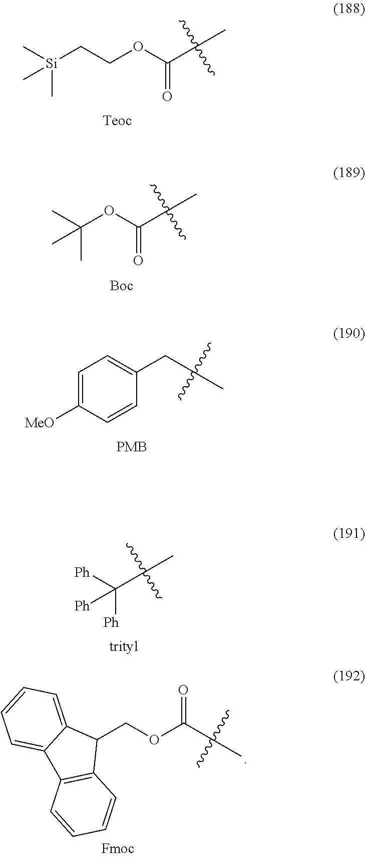

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods

The present invention includes methods for treatment and/or prevention of cancer. As provided herein, compositions comprising a weakly acidic prodrug and a traceless linker can be used to treat or prevent cancer in a patient in need thereof. In certain embodiments, the methods of the present invention comprise administering at least one prodrug compound of the invention alone, or in combination with other agents that modulate a particular pathological process. For example, prodrug compounds of the invention can be administered in combination with one or more additional anticancer agents. As used herein, two agents are said to be administered in combination when the two agents are administered simultaneously or are administered independently in a fashion such that the agents act at the approximately same time.

Examples of the cancer include, but are not limited to, squamous cell cancer (e.g., epithelial squamous cell cancer), melanoma, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, gastrointestinal stromal tumors, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, testicular cancer, hepatic carcinoma, anal carcinoma, penile carcinoma, mouth and throat cancer as well as head and neck cancer. In some embodiments the cancer is a carcinoma or sarcoma. In various embodiments, the cancer is a solid tumor, as these produce the most strongly acidic tumor microenvironment. In various embodiments, solid tumors can be defined to include certain circumstances of otherwise non-solid cancer cell masses, such as lymphoma building up as quasi-solid masses in lymph nodes and similar collection areas in the body.

The dosage administered will be dependent upon the age, health, and weight of the recipient, kind of concurrent treatment, if any, frequency of treatment, and the nature of the effect desired.

### Combination Therapies

The compounds useful within the methods of the invention may be used in combination with one or more additional therapeutic agents useful for treating a cancer. These additional therapeutic agents may comprise compounds that are commercially available or synthetically accessible to those skilled in the art. These additional therapeutic agents are known to treat, prevent, or reduce the symptoms, of a cancer.

In certain embodiments, administering the compound of the invention to the subject allows for administering a lower dose of the additional therapeutic agent as compared to the dose of the additional therapeutic agent alone that is required to achieve similar results in treating or preventing a cancer in the subject. For example, in certain embodiments, the compound of the invention enhances the anticancer activity of the additional therapeutic compound, thereby allowing for a lower dose of the additional therapeutic compound to provide the same effect. In other embodiments, administering the compound of the invention to the subject in addition to administering an additional therapeutic agent achieves superior results in treating or preventing a cancer as compared to the additional therapeutic agent alone.

In certain embodiments, the compounds of the present invention are used in combination with radiation therapy. In other embodiments, the combination of administration of the compounds of the present invention and application of radiation therapy is more effective in treating or preventing cancer than application of radiation therapy by itself. In yet other embodiments, the combination of administration of the compounds of the present invention and application of radiation therapy allows for use of lower amount of radiation therapy in treating the subject.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a cancer in a patient.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a cancer in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present invention also includes a multi-layer tablet comprising a layer providing for the delayed release of one or more compounds of the invention, and a further layer providing for the immediate release of a medication for treatment of G-protein receptor-related diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

In various embodiments, the compounds of the invention may be delivered transdermally. In various embodiments, this may be appropriate when the solid tumor is near or on the surface of the patient's skin, by way of non-limiting example, melanoma and squamous cell skin cancer and head and neck cancers. In various embodiments, the transdermal delivery formulation may contain one or more penetration enhancers.

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer than the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a cancer in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which can be expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### SYNTHETIC EXAMPLES

Starting materials were used as received unless otherwise noted. All moisture sensitive reactions were performed in an inert, dry atmosphere of nitrogen in oven dried glassware. Reagent grade solvents were used for extractions and flash chromatography. 3 Å molecular sieve was activated at 135° C. for 12 hours before use. Reaction progress was monitored by LC-MS analyses performed on a Waters UPLC/MS instrument equipped with a RP-C18 column (1.7 μm particle size, 2.1×50 mm), dual atmospheric pressure chemical ionization (API)/electrospray (ESI) mass spectrometry detector, and photodiode array detector. Flash column chromatography was performed using RediSepRf NP-silica (40-63 μm 60 Å) or Teledyne RediSepRf Gold RP-C18 column (20-40 μm 100 Å) in Teledyne ISCO CombiFlash Rf 200 purification system unless otherwise specified. The solvent compositions reported for all chromatographic separations are on a volume/volume (v/v) basis. Infrared (IR) spectra were recorded on a Thermo Nicolet 6700 FT-IR Spectrometer. ¹H-NMR spectra were recorded on Agilent DD2 400 MHz, 500 MHz, 600 MHz spectrometer and reported in parts per million (ppm) on the 6 scale relative to CDCl₃ (δ 7.26), Methanol-d4 (δ 3.31), ACN-d3 (δ 1.94), D₂O (δ 4.79) as an internal standard. Data are reported as follows: chemical shift, multiplicity (s=singlet, d=doublet, t=triplet, q=quartet, br=broad, m=multiplet), coupling constants (Hz), and integration. ¹³C-NMR spectra were recorded on Agilent DD2 125 MHz, and 150 MHz spectrometers and were reported in parts per million (ppm) on the 6 scale relative to CDCl₃ (δ 77.00), Methanol-d4 (δ 49.00), ACN-d3 (δ 1.32).

Reaction procedures for compound (121), (122) and (123) were synthesized according to the procedure reported in Chem. Eur. J. 2006, 12:3655-3671 2 and European Journal of Medicinal Chemistry 2014, 82, 355 with minor modifications.

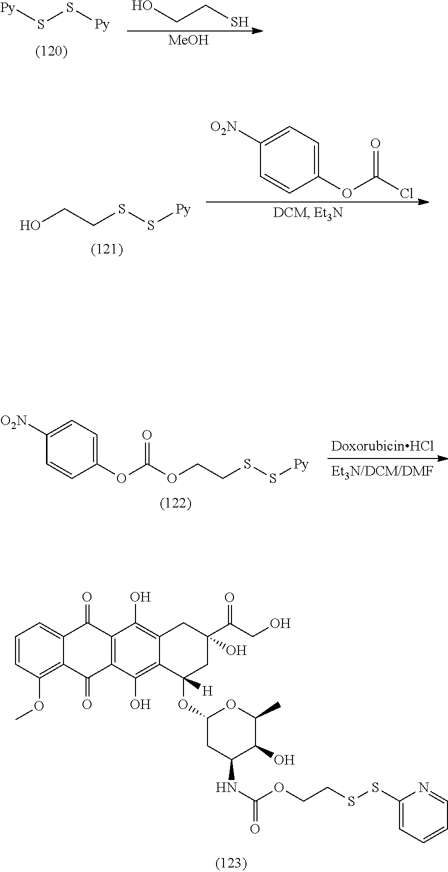

To an oven dried round bottom flask under nitrogen at room temperature equipped with a stir bar was added 2′-aldrithiol (6.4 g, 29.0 mmol) in 25 mL methanol purged with nitrogen. To this mixture was added 2-mercaptoethanol dropwise (757 mg, 9.78 mmol). The solution turned yellow and was allowed to stir for three hours. The solvent was then removed in vacuo and the crude material was purified by column chromatography (SiO₂, DCM:EtOAc=4:1). The excess amount of the 2′-aldrithiol was eluted first with strong 280 nm wavelength absorption, followed by the desired product and then 2-mercaptopyridine. The product was a yellow oil (5.05 g, 27.0 mmol, 93% yield). LCMS (M+1)=188.274. NMR was consistent with the reported number. 2-(2-pyridyldithio)ethanol (121) (3 g, 16 mmol) and Et₃N (4.5 mL, 32 mmol) were dissolved in DCM (60 mL) at 0° C. 4-Nitrophenyl chloroformate (3.55 g, 17.6 mmol) was added to the above solution at the same temperature. The resulting light yellow solution was allowed to warm up to room temperature and stirred for another 4 hours. LCMS analysis for the above mixture after 4 hours stirring indicated complete consumption of the starting material. The light yellow reaction mixture was washed with water (2×50 mL) to remove the precipitated Et₃N hydrochloride. The organic layer was dried with Na₂SO₄, filtered, and concentrated under vacuum. Crude product (122) was purified by column chromatography (SiO₂, Hexane:EtOAc=1:1) as a light yellow oil (5.08 g, 14.4 mmol, 90%). NMR was consistent with the published data. Compound (122) (1.34 g, 3.79 mmol) and Et₃N (0.72 mL, 5.17 mmol) were dissolved in DCM/DMF (1:1, 12 mL, for the poor solubility of doxorubicin hydrochloride in DCM). Doxorubicin hydrochloride (2 g, 3.45 mmol) was then added to the above solution and the overall reaction mixture was stirred at room temperature under dark for 12 hours. LCMS analysis for the above mixture after 12 hours stirring indicated complete consumption of compound (122). The solvent was removed under vacuum and the crude material was re-dissolved in DCM and purified by column chromatography (SiO₂, DCM:MeOH=20:1) to afford product (123) as a red solid (2.27 g, 30.0 mmol, 87%).^{2 1}H NMR (500 MHz, DMSO-d₆) δ 13.88 (s, 1H), 13.11 (s, 1H), 8.56-8.16 (m, 1H), 7.99-7.61 (m, 4H), 7.49 (d, J=8.4 Hz, 1H), 7.25-7.02 (m, 1H), 6.80 (d, J=8.0 Hz, 1H), 5.34 (s, 1H), 5.27-5.08 (m, 1H), 4.86 (t, J=5.9 Hz, 1H), 4.83-4.79 (m, 1H), 4.70 (d, J=5.7 Hz, 1H), 4.57 (d, J=5.9 Hz, 3H), 4.34-4.00 (m, 3H), 3.90 (s, 3H), 3.68 (d, J=13.4 Hz, 1H), 3.42 (dd, J=6.0, 2.6 Hz, 1H), 3.12-2.71 (m, 4H), 2.48 (t, J=1.9 Hz, 1H), 2.18 (d, J=14.1 Hz, 1H), 2.03 (dd, J=14.2, 5.5 Hz, 1H), 1.92-1.73 (m, 1H), 1.46 (d, J=11.1 Hz, 1H), 1.12 (d, J=6.3 Hz, 3H); 13C NMR (126 MHz, DMSO-d₆) δ 214.33, 186.56, 186.44, 161.04, 159.38, 156.48, 155.41, 154.87, 149.92, 138.19, 136.45, 135.70, 134.77, 134.32, 121.60, 120.09, 119.90, 119.62, 119.23, 110.94, 110.81, 100.81, 75.31, 70.20, 68.39, 67.06, 64.20, 61.85, 56.89, 47.58, 37.79, 36.76, 32.39, 30.24, 17.47.

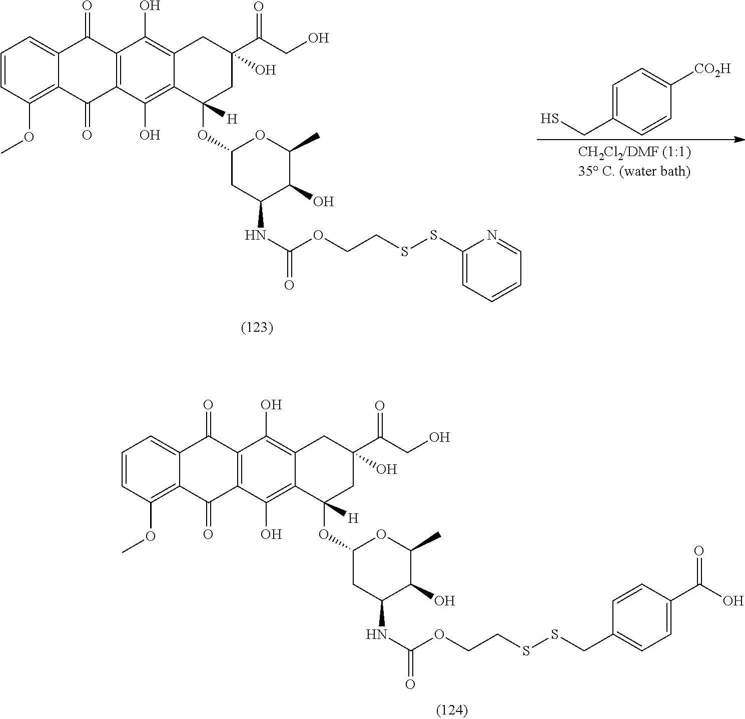 [SMILES: COc1cccc2c1C(=O)c1c(O)c3c(c(O)c1C2=O)C[C@@](O)(C(=O)CO)C[C@@H]3O[C@H]1C[C@H](NC(=O)OCCSSc2ccccn2)[C@H](O)[C@H](C)O1 ; O=C(O)c1ccc(CS)cc1 ; COc1cccc2c1C(=O)c1c(O)c3c(c(O)c1C2=O)C[C@@](O)(C(=O)CO)C[C@@H]3O[C@H]1C[C@H](NC(=O)OCCSSCc2ccc(C(=O)O)cc2)[C@H](O)[C@H](C)O1]

The Dox-SS-Py (123) (336 mg, 0.44 mmol) and 4-(mercaptomethyl)benzoic acid (94 mg, 0.55 mmol) were dissolved in a mixture of DCM and DMF (2 mL+2 mL). The reaction was stirred at 35° C. water bath for 12 hours. After 12 h, LCMS indicated all Dox-SS-Py was consumed. DCM was removed under vacuum and the remaining solution was diluted with 4 mL of MeOH and 1 mL of water. The crude mixture was purified by reverse phase HPLC (20-50% MeCN in H₂O with 0.1% formic acid as buffer over 24 mins) to obtain the pure prodrug (124) (188 mg, 0.23 mmol, 52%) as a red solid. ¹H NMR (600 MHz, DMSO-d₆) δ 13.96 (s, 1H), 13.22 (bs, 2H), 7.95-7.73 (m, 5H), 7.57 (d, J=7.7 Hz, 1H), 7.35 (d, J=7.8 Hz, 2H), 6.79 (d, J=8.0 Hz, 1H), 5.17 (d, J=3.7 Hz, 1H), 4.88 (t, J=4.4 Hz, 1H), 4.54 (s, 2H), 4.12-3.98 (m, 3H), 3.95 (s, 2H), 3.93 (s, 3H), 3.72-3.57 (m, 1H), 3.41 (s, 1H), 3.02-2.79 (m, 2H), 2.65 (t, J=6.6 Hz, 2H), 2.17 (d, J=15.5 Hz, 1H), 2.13-2.02 (m, 1H), 1.80 (td, J=13.1, 3.9 Hz, 1H), 1.44 (dd, J=12.4, 4.4 Hz, 1H), 1.09 (d, J=6.4 Hz, 3H); ¹³C NMR (151 MHz, dmso) δ 214.29, 186.75, 168.34, 165.31, 161.15, 156.54, 155.48, 155.05, 141.31, 136.58, 135.90, 135.08, 134.60, 129.70, 129.42, 120.36, 120.04, 119.38, 111.15, 110.99, 100.70, 75.37, 70.29, 68.37, 67.10, 64.11, 62.03, 56.98, 47.57, 41.97, 37.04, 36.79, 32.51, 30.24, 17.45; HR-MS: (M+Na)⁺=836.1888 (experimental); exact mass=836.1653 (theoretical).

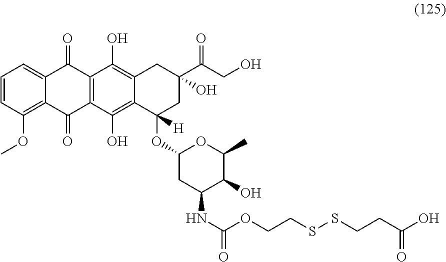 [SMILES: COc1cccc2c1C(=O)c1c(O)c3c(c(O)c1C2=O)C[C@@](O)(C(=O)CO)C[C@@H]3O[C@H]1C[C@H](NC(=O)OCCSSCCC(=O)O)[C@H](O)[C@H](C)O1]

The Dox-SS-Py (123) (594 mg, 0.74 mmol) and 3-mercaptopropanoic acid (100 mg, 0.94 mmol) were dissolved in a mixture of DCM and DMF (3 mL+3 mL). The reaction was stirred at 35° C. water bath for 12 hours. After 12 h, LCMS indicated small amount of Dox-SS-Py remained with almost the same retention time in LCMS as the desired prodrug. Another 50 mg of the 3-mercaptopropanoic acid were added and the reaction was stirred for another 2 h. After 2 h, LCMS indicated all Dox-SS-Py was consumed. DCM was removed under vacuum and the remaining solution was diluted with 5 mL of MeOH and 2 mL of water. The crude mixture was purified by reverse phase HPLC (20-60% MeCN in H₂O with 0.1% formic acid as buffer over 22 mins) to obtain the pure prodrug YU241526 (125) (323 mg, 0.43 mmol, 58%) as a red solid. ¹H NMR (500 MHz, DMSO-d₆) δ 13.91 (s, 1H), 13.15 (s, 1H), 7.93-7.65 (m, 2H), 7.53 (d, J=8.7 Hz, 1H), 6.77 (d, J=7.9 Hz, 1H), 5.32-5.10 (m, 1H), 4.85 (s, 1H), 4.57 (s, 2H), 4.34-4.01 (m, 3H), 3.92 (s, 3H), 3.78-3.65 (m, 1H), 3.43 (s, 1H), 3.12-2.73 (m, 6H), 2.56 (t, J=6.9 Hz, 2H), 2.18 (d, J=14.2 Hz, 1H), 2.05 (dd, J=14.4, 5.3 Hz, 1H), 1.91-1.76 (m, 1H), 1.46 (dd, J=12.8, 4.3 Hz, 1H), 1.11 (d, J=6.4 Hz, 3H); 13C NMR (126 MHz, DMSO-d₆) δ 214.26, 186.70, 186.57, 173.10, 161.11, 156.49, 155.50, 154.90, 136.50, 135.80, 134.89, 134.40, 120.23, 119.99, 119.30, 111.04, 110.91, 100.76, 75.34, 70.20, 68.38, 67.09, 64.17, 62.16, 56.94, 47.58, 37.33, 36.86, 34.05, 33.58, 32.45, 30.23, 17.46; HR-MS: (M+Na)⁺=774.1706 (experimental); exact mass=774.1706 (theoretical).

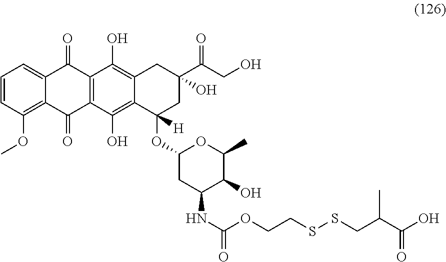 [SMILES: COc1cccc2c1C(=O)c1c(O)c3c(c(O)c1C2=O)C[C@@](O)(C(=O)CO)C[C@@H]3O[C@H]1C[C@H](NC(=O)OCCSSCC(C)C(=O)O)[C@H](O)[C@H](C)O1]

The Dox-SS-Py (123) (535 mg, 0.70 mmol) and 3-mercapto-2-methylpropanoic acid (102 mg, 0.84 mmol) were dissolved in a mixture of DCM and DMF (3 mL+3 mL). The reaction was stirred at 35° C. water bath for 12 hours. After 12 h, another 60 mg of the 3-mercapto-2-methylpropanoic acid were added and the reaction was stirred for another 4 h. After 4 h, LCMS indicated all Dox-SS-Py was consumed. DCM was removed under vacuum, and the remaining solution was diluted with 5 mL of MeOH and 2 mL of water. The crude mixture was purified by reverse phase HPLC (20-40% MeCN in H₂O with 0.1% formic acid as buffer over 31 mins) to obtain the pure prodrug YU241527 (126) (306 mg, 0.40 mmol, 57%) as a red solid. ¹H NMR (500 MHz, DMSO-d₆) δ 13.90 (s, 1H), 13.14 (s, 1H), 7.78 (dt, J=15.6, 7.6 Hz, 2H), 7.52 (d, J=8.2 Hz, 1H), 6.77 (d, J=7.9 Hz, 1H), 5.35 (s, 1H), 5.18 (d, J=3.6 Hz, 1H), 4.85 (t, J=4.1 Hz, 1H), 4.57 (s, 2H), 4.23-4.01 (m, 3H), 3.92 (s, 3H), 3.75-3.59 (m, 1H), 3.43 (s, 1H), 3.02-2.77 (m, 5H), 2.76-2.56 (m, 2H), 2.18 (d, J=14.0 Hz, 1H), 2.05 (dd, J=14.4, 5.5 Hz, 1H), 1.82 (td, J=13.0, 3.9 Hz, 1H), 1.46 (dd, J=12.7, 4.4 Hz, 1H), 1.26-1.01 (m, 6H); ¹³C NMR (126 MHz, DMSO-d₆) δ 214.26, 186.68, 186.55, 176.12, 161.11, 156.49, 155.50, 154.89, 136.50, 135.79, 134.86, 134.39, 120.20, 119.99, 119.29, 111.02, 110.89, 100.77, 75.34, 70.20, 68.38, 67.10, 64.17, 62.14, 56.93, 47.58, 42.10, 39.34, 37.31, 36.84, 32.45, 30.24, 17.46, 16.81; HR-MS: (M+Na)⁺=788.1888 (experimental); exact mass=788.1653 (theoretical).

The Dox-SS-Py (123) (350 mg, 0.46 mmol) and 8-mercaptooctanoic acid (103 mg, 0.58 mmol) were dissolved in a mixture of DCM and DMF (3 mL+3 mL). The reaction was stirred at 35° C. water bath for 12 hours. After 12 h, another 30 mg of the 8-mercaptooctanoic acid was added and the reaction was stirred for another 4 h. After 4 h, small amount of the Dox-SS-Py was still observed. Another 30 mg of the 8-mercaptooctanoic acid were added. Reaction mixture was stirred for another 6 h, DCM was then removed under vacuum and the remaining solution was diluted with 5 mL of MeOH and 1.5 mL of water. The crude mixture was purified by reverse phase HPLC (30-60% MeCN in H₂O with 0.1% formic acid as buffer over 32 mins) to obtain the pure prodrug YU241531 (41):

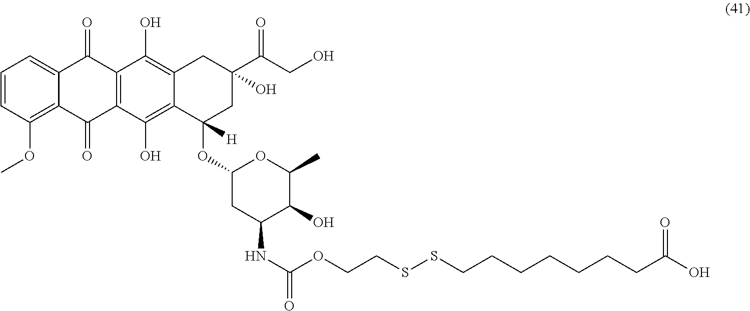 [SMILES: COc1cccc2c1C(=O)c1c(O)c3c(c(O)c1C2=O)C[C@@](O)(C(=O)CO)C[C@@H]3O[C@H]1C[C@H](NC(=O)OCCSSCCCCCCCC(=O)O)[C@H](O)[C@H](C)O1]

(183 mg, 0.22 mmol, 48%) as a red solid. ¹H NMR (500 MHz, CDCl₃) δ 13.88 (s, 1H), 13.12 (s, 1H), 8.21-7.82 (m, 2H), 7.82-7.60 (m, 1H), 7.51-7.30 (m, 1H), 5.88-4.93 (m, 3H), 4.75 (s, 2H), 4.22-4.12 (m, 3H), 4.05 (s, 3H), 3.83 (bs, 1H), 3.70-3.63 (m, 1H), 3.20-3.11 (m, 1H), 2.97-2.73 (m, 2H), 2.66 (t, J=7.3 Hz, 2H), 2.39-2.19 (m, 2H), 2.17-2.08 (m, 1H), 1.89-1.75 (m, 1H), 1.71-1.55 (m, 5H), 1.44-1.17 (m, 11H); 13C NMR (126 MHz, CDCl₃) δ 213.81, 186.83, 186.45, 179.31, 178.71, 171.26, 164.43, 160.94, 156.14, 155.47, 135.74, 135.30, 133.56, 120.65, 119.77, 118.47, 111.43, 100.67, 69.57, 67.34, 65.47, 63.01, 60.43, 56.61, 39.07, 33.92, 33.83, 29.67, 29.05, 28.83, 28.76, 28.71, 28.63, 28.22, 24.57, 24.51, 22.66, 16.84, 16.79; HR-MS: (M+H)⁺=822.1586 (experimental); exact mass=822.2460 (theoretical).

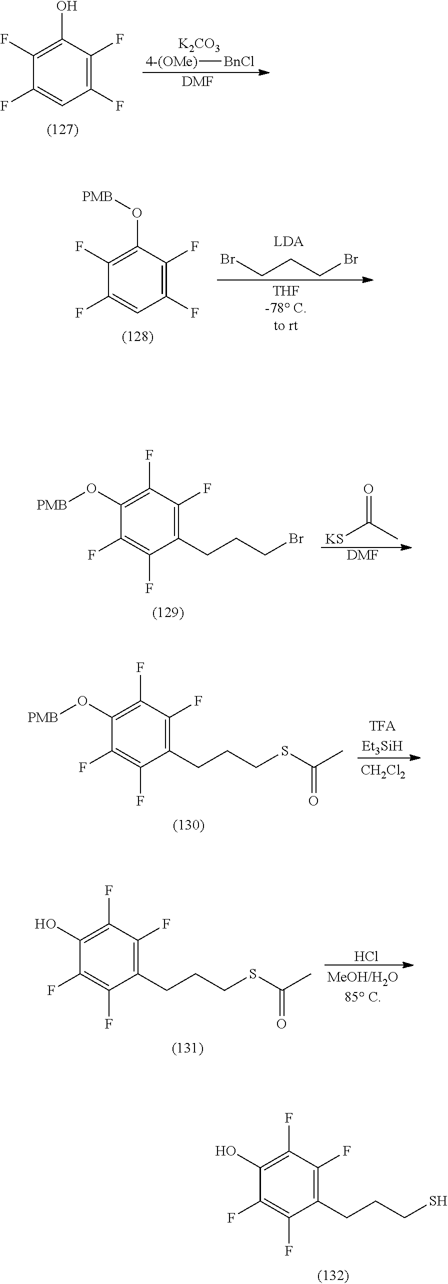

Step 1: Synthesis of 1,2,4,5-tetrafluoro-3-((4-methoxybenzyl)oxy)benzene (128). To a 50 mL round bottom flask equipped with magnetic stir bar is added 2,3,5,6-tetrafluorophenol (2 g, 12.04 mmol), K₂CO₃ (3.33 g, 24.09 mmol), and DMF (15 mL). To this stirring mixture is added 4-methoxy-benzoyl chloride (1.89 g, 12.04 mmol, 1.63 mL) dropwise and the reaction mixture is stirred overnight at room temperature under nitrogen. The solvent is removed under reduced pressure to give a solid residue that is then partitioned between layers of EtOAc and water. The aqueous layer is separated and extracted with EtOAc (3×). The combined organic layer is washed with brine and dried with MgSO₄, and the drying agent is removed by vacuum filtration. The filtrate is concentrated to dryness to afford crude material as yellow oil. For purification, the crude material is loaded onto the SNAP Ultra 50 g silica gel column and the product is eluted with 0-20% EtOAc in hexanes gradient. Removal of solvent under reduced pressure provided 4-methoxybenzyl ether product (128) (2.76 g, 80%) as crystalline white solid. ¹H NMR (400 MHz, Chloroform-d₃) δ 7.39-7.30 (m, 2H), 6.93-6.85 (m, 2H), 6.74 (tt, J=10.0, 7.0 Hz, 1H), 5.19 (s, 2H), 3.81 (s, 3H); ¹⁹F NMR (376 MHz, Chloroform-d) 6-140.13 to −140.27 (m, 2F), −155.87 to −155.98 (m, 2F).

Step 2: Synthesis of 1-(3-bromopropyl)-2,3,5,6-tetrafluoro-4-((4-methoxybenzyl)oxy)benzene (129). To a 25 mL round bottom flask flushed with nitrogen and equipped with a magnetic stir bar is weighed out 1,2,4,5-tetrafluoro-3-[(4-methoxyphenyl)methoxy]benzene (128) (1 g, 3.49 mmol) and dissolved with THF (20 mL). The solution is stirred under nitrogen and then cooled down to −78° C. over 15 minutes. To this solution at −78° C. with stirring is then added LDA (1 M, 4.19 mL) as a solution in THF and stirred for 1 h at −78° C. After 1 h, a solution of 1,3-dibromopropane (1.41 g, 6.99 mmol, 694.93 uL) in 1 mL THF is added to the mixture at −78° C. and warmed up to room temperature with stirring over 1 h. Reaction is monitored by ¹⁹F NMR, and after stirring for addition 3 h at room temperature the reaction is quenched by addition of water. The reaction mixture is then partitioned between layers of EtOAc and water. The aqueous layer is separated and extracted with EtOAc (3×). Combined organic layer is washed with brine and dried with sodium sulfate, and the drying agent is removed by vacuum filtration. The filtrate is concentrated down before loaded onto the SNAP Ultra 50 g silica gel column and the product is eluted with 0-10% EtOAc in hexanes gradient. Removal of solvents under reduced pressure afforded alkyl bromide product (129) (710 mg, 50%) as off-white solid. ¹H NMR (400 MHz, Chloroform-d) δ 7.37-7.32 (m, 2H), 6.92-6.86 (m, 2H), 5.15 (s, 2H), 3.81 (s, 3H), 3.39 (t, J=6.7 Hz, 2H), 2.82 (t, J=7.5 Hz, 2H), 2.13 (p, J=13.9, 6.8 Hz, 2H); ¹⁹F NMR (376 MHz, Chloroform-d) 6-145.56 to −145.70 (m, 2F), −156.43 to −156.53 (m, 2F).

Step 3: Synthesis of S-(3-(2,3,5,6-tetrafluoro-4-((4-methoxybenzyl)oxy)phenyl)propyl) ethanethioate (130). To a 50 mL round bottom flask equipped with a magnetic stir bar is added 1-(3-bromopropyl)-2,3,5,6-tetrafluoro-4-[(4-methoxyphenyl)methoxy]benzene (129) (300 mg, 736.74 umol) then dissolved with DMF (5 mL). To this stirring solution is added potassium thioacetate (168.28 mg, 1.47 mmol) and the reaction mixture is stirred at room temperature for 4 h. Monitoring with TLC (10% EtOAc in hexanes) shows complete consumption of starting material. The reaction mixture is then partitioned between layers of EtOAc and. The aqueous layer is separated and extracted with EtOAc (3×). Combined organic layer is washed with brine (3×) to remove DMF and dried with MgSO₄ before the drying agent is removed via vacuum filtration. The filtrate is concentrated to dryness to give crude material as an oil that is then directly loaded onto a SNAP Ultra 25 g silica gel column. The product is eluted with 0-15% EtOAc in hexanes gradient and the solvents are removed under reduced pressure to afford the thioacetate product (130) (269 mg, 91%) as white solid. ¹H NMR (400 MHz, Chloroform-d) δ 7.38-7.30 (m, 2H), 6.93-6.84 (m, 2H), 5.14 (s, 2H), 3.81 (s, 3H), 2.88 (t, J=7.3 Hz, 2H), 2.73 (t, J=7.6 Hz, 2H), 2.33 (s, 3H), 1.85 (p, J=7.5 Hz, 2H).

Step 4: S-(3-(2,3,5,6-tetrafluoro-4-hydroxyphenyl)propyl) ethanethioate (131). To a 25 mL round bottom flask is added S-[3-[2,3,5,6-tetrafluoro-4-[(4-methoxyphenyl)methoxy]phenyl]propyl] ethanethioate (130) (269 mg, 668.49 umol), Triethylsilane (93.28 mg, 802.18 umol, 128.13 uL), and CH₂Cl₂ (3 mL). To this stirring solution at room temperature is added TFA (1.48 g, 12.98 mmol, 1 mL) and the reaction mixture is stirred for 1 h at ambient temperature. The reaction is monitored by TLC for consumption of starting material, and after 3 h the reaction was concentrated under reduced pressure and loaded onto a SNAP Ultra 10 g silica gel column. The product is eluted with 0-30% EtOAc in hexanes gradient and the solvents are removed under reduced pressure to afford phenol product (131) (146 mg, 77%) as white solid. ¹H NMR (400 MHz, Chloroform-d) δ 5.36 (bs, 1H), 2.89 (t, J=7.3 Hz, 2H), 2.73 (t, J=7.6 Hz, 2H), 2.34 (s, 3H), 1.86 (p, J=7.5 Hz, 2H).

Step 5: 2,3,5,6-tetrafluoro-4-(3-mercaptopropyl)phenol (132). To a 25 mL round bottom flask equipped with magnetic stir bar is added S-[3-(2,3,5,6-tetrafluoro-4-hydroxy-phenyl)propyl] ethanethioate (131) (40 mg, 141.72 umol) and dissolved with degassed (bubbled with N₂) MeOH (2 mL). To this stirring solution is added an aqueous solution of HCl (1 M, 1 mL) and the flask is then equipped with a reflux condenser and placed under nitrogen atmosphere. The reaction mixture is heated to 85° C. and monitored by LC-MS for consumption of starting material. After stirring at 85° C. overnight under nitrogen the reaction is cooled down to room temperature before partitioned between layers of water and CH₂Cl₂. The aqueous layer is separated and extracted with CH₂Cl₂ (3×). The combined organic layer is dried with MgSO₄ and the drying agent is removed by vacuum filtration. The filtrate is concentrated under reduced pressure to afford crude thiol product (132) (32.8 mg, 96%) as colorless oil that was sufficiently clean by ¹H NMR for use in next step without further purifications. ¹H NMR (400 MHz, Chloroform-d) δ 5.49 (bs, 1H), 2.80-2.75 (m, 2H), 2.54 (q, J=7.5 Hz, 2H), 1.89 (p, J=7.3 Hz, 2H), 1.41 (t, J=8.0 Hz, 1H).

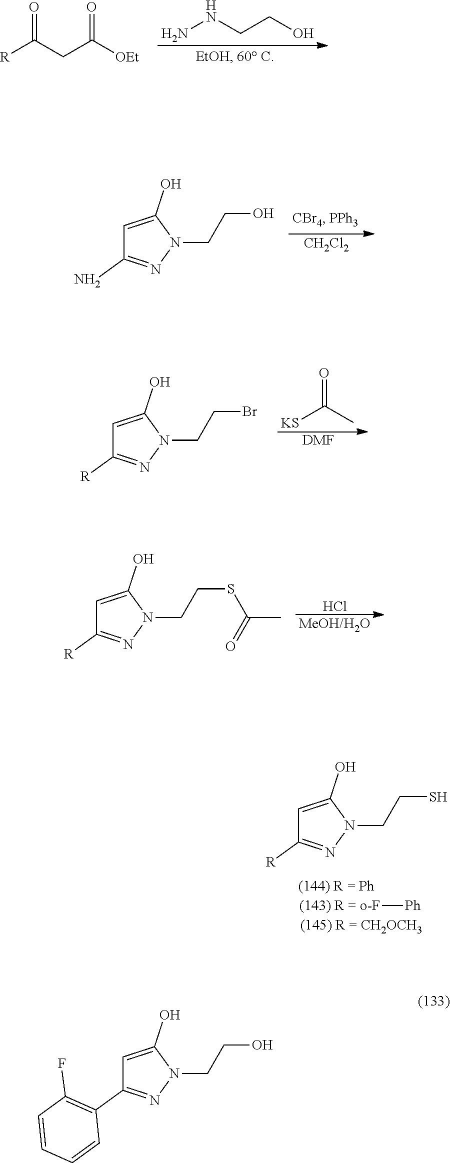

Step 1: Synthesis of 3-(2-fluorophenyl)-1-(2-hydroxyethyl)-1H-pyrazol-5-ol (133). To a 100 mL round bottom flask equipped with magnetic stir bar is added reagent grade EtOH (20 mL) followed by ethyl 3-(2-fluorophenyl)-3-oxo-propanoate (134) (2 g, 9.51 mmol, 1.72 mL) with stirring. To this stirring solution at room temperature is added 2-Hydroxyethylhydrazine (760.24 mg, 9.99 mmol, 678.79 uL) before an air condenser is attached and the solution heated to 60° C. for 2 h where the reaction is monitored by LC-MS. After cooling down to room temperature, the reaction mixture is partitioned between layers of EtOAc and water. The aqueous layer is separated and acidified with minimal amounts of 1N HCl aqueous solution until pH=1 before extracted with EtOAc (3×). Combined organic layer is washed with brine and dried with MgSO₄. Drying agent is removed by vacuum filtration and the filtrate is concentrated under reduced pressure before being loaded onto a SNAP Ultra 50 g silical gel column. The product is eluted with 0-20% MeOH in CH₂Cl₂ gradient and the solvents are removed under reduced pressure to afford pyrazolone product (133) (1.12 g, 53%) as light yellow solid. LC-MS: t_{R}=2.9 min, m/z=223.7 (ESI/[M+H]⁺).

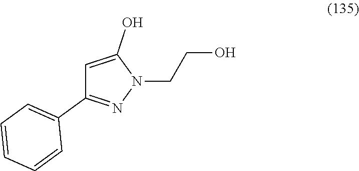 [SMILES: OCCn1nc(-c2ccccc2)cc1O]

Synthesis of 1-(2-hydroxyethyl)-3-phenyl-1H-pyrazol-5-ol (68). The general procedure for (133) is followed using ethyl 3-oxo-3-phenylpropanoate (1 g, 5.20 mmol, 0.9 mL). Flash column chromatography purification using SNAP Ultra 25 g silica column and 0-15% MeOH in CH₂Cl₂ gradient provided pyrazolone product (135) (250 mg, 24%) as light yellow solid. LC-MS: t_{R}=3.0 min, m/z=205.1 (ESI/[M+H]⁺).

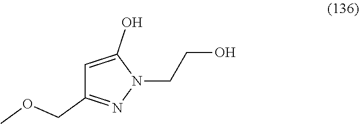

Synthesis of 1-(2-hydroxyethyl)-3-(methoxymethyl)-1H-pyrazol-5-ol (136). The general procedure of (133) is followed using ethyl 4-methoxy-3-oxobutanoate (1 g, 6.24 mmol). Flash column chromatography purification using SNAP Ultra 50 g silical column and 0-30% MeOH in CH₂Cl₂ gradient provided pyrazolone product (136) (300 mg, 28%). LC-MS: t_{R}=1.5 min, m/z=173.1 (ESI/[M+H]⁺).

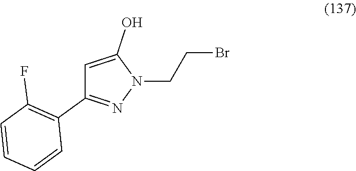 [SMILES: Oc1cc(-c2ccccc2F)nn1CCBr]

Step 2: Synthesis of 1-(2-bromoethyl)-3-(2-fluorophenyl)-1H-pyrazol-5-ol (137). To a 25 mL round bottom flask containing a solution of 5-(2-fluorophenyl)-2-(2-hydroxyethyl)pyrazol-3-ol (133) (1.12 g, 5.04 mmol) in CH₂Cl₂ (10 mL) is added CBr₄ (1.84 g, 5.54 mmol) followed by PPh₃ (1.45 g, 5.54 mmol) with stirring. The reaction is warmed up with water bath to 35° C. and stirred under nitrogen overnight. After stirring overnight, the reaction is monitored by LC-MS before it is concentrated down to dryness. The resulting crude material residue is solubilized with minimal CH₂Cl₂ before being loaded onto a SNAP Ultra 50 g silica gel column. The product is eluted with 0-10% MeOH in CH₂Cl₂ gradient and the solvents are removed under reduced pressure to provide alkyl bromide (137) (378 mg, 26%) with >90% purity. LC-MS: t_{R}=3.4 min, m/z=285.0 (ESI/[M+H]⁺).

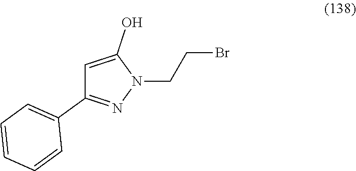 [SMILES: Oc1cc(-c2ccccc2)nn1CCBr]

Synthesis of 1-(2-bromoethyl)-3-phenyl-1H-pyrazol-5-ol (138). The general procedure for (137) is followed using (135) (300 mg, 1.47 mmol). Flash chromatography purification using SNAP Ultra 25 g silica gel column and 0-10% MeOH in CH₂Cl₂ gradient delivered alkyl bromide (138) as desired product (93 mg, 24%). LC-MS: t_{R}=3.4 min, m/z=267.0 (ESI/[M+H]⁺).

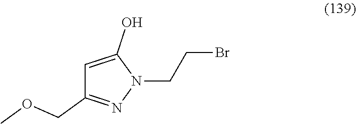

Synthesis of 1-(2-bromoethyl)-3-(methoxymethyl)-1H-pyrazol-5-ol (139). The general procedure for (137) is followed using (136) (300 mg, 1.74 mmol). Flash chromatography purification using SNAP Ultra 25 g silica gel column delivered a mixture of desired product (139) with some impurities. This mixture was taken to next step without further purifications. LC-MS: t_{R}=2.7 min, m/z=235.0 (ESI/[M+H]⁺).

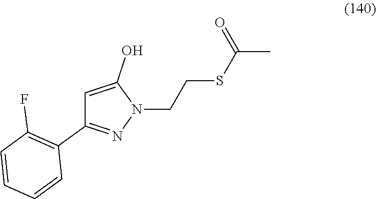 [SMILES: CC(=O)SCCn1nc(-c2ccccc2F)cc1O]

Step 3: Synthesis of S-(2-(3-(2-fluorophenyl)-5-hydroxy-1H-pyrazol-1-yl)ethyl) ethanethioate (140). To a 100 mL round bottom flask equipped with magnetic stir bar and containing 2-(2-bromoethyl)-5-(2-fluorophenyl)pyrazol-3-ol (137) (378 mg, 1.33 mmol) is added DMF (5 mL). To this stirring solution is then added potassium thioacetate (303.79 mg, 2.66 mmol). The reaction mixture is stirred at room temperature with reaction progress monitored by LC-MS. After 1 h of stirring at room temperature, all starting material (137) has been consumed. The reaction mixture is then suspended between layers of EtOAc and water. The aqueous layer is separated and acidified with minimal amounts of 1N HCl aqueous solution to pH=1 before extracted with EtOAc (3×). Combined organic layer is washed with brine and dried with MgSO₄. The drying agent is removed by vacuum filtration and the filtrate is concentrated down before loaded onto a SNAP Ultra 10 g silica gel column. The product is eluted with 20-100% EtOAc in hexanes gradient and the solvents are removed under reduced pressure to afford thioacetate product (140) (199 mg, 53%) as white solid. ¹H NMR (400 MHz, Chloroform-d) δ 7.97 (td, J=7.7, 1.8 Hz, 1H), 7.43-7.36 (m, 1H), 7.21 (td, J=7.6, 1.2 Hz, 1H), 7.12 (ddd, J=11.5, 8.3, 1.2 Hz, 1H), 3.98 (t, J=6.6 Hz, 2H), 3.74 (d, J=2.9 Hz, 2H), 3.27 (t, J=6.6 Hz, 2H), 2.34 (s, 3H). LC-MS: t_{R}=3.4 min, m/z=281.0 (ESI/[M+H]⁺).

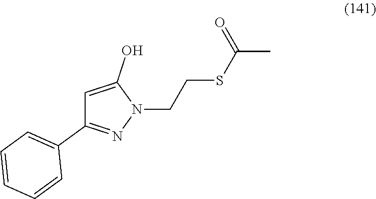 [SMILES: CC(=O)SCCn1nc(-c2ccccc2)cc1O]

Synthesis of S-(2-(5-hydroxy-3-phenyl-1H-pyrazol-1-yl)ethyl) ethanethioate (141). The general procedure for (140) is followed using (138) (93 mg, 348 umol). Flash column chromatography purification using a SNAP Ultra 10 g silica gel column provided the desired thioacetate product (141) (47 mg, 51%) as white solid. LC-MS: t_{R}=3.3 min, m/z=263.0 (ESI/[M+H]⁺).

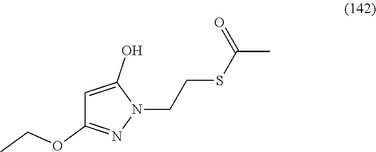

Synthesis of S-(2-(5-hydroxy-3-(methoxymethyl)-1H-pyrazol-1-yl)ethyl) ethanethioate (142). The general procedure for (140) is followed using (139) mixture (300 mg, 1.28 mmol). Flash column chromatography purification using a SNAP Ultra C18 30 g (reverse phase) column and 10-100% MeCN in water (0.1% formic acid) gradient provided the desired thioacetate product (142) (54 mg, 18%) as white solid. LC-MS: t_{R}=2.8 min, m/z=231.1 (ESI/[M+H]⁺).

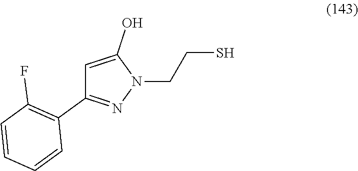 [SMILES: Oc1cc(-c2ccccc2F)nn1CCS]

Step 4: Synthesis of 3-(2-fluorophenyl)-1-(2-mercaptoethyl)-1H-pyrazol-5-ol (143). To a 50 mL round bottom flask equipped with magnetic stir bar is weighed out S-[2-[3-(2-fluorophenyl)-5-hydroxy-pyrazol-1-yl]ethyl] ethanethioate (140) (51 mg, 181.94 umol) before dissolved with degassed (bubbled with nitrogen) MeOH (3 mL). To this stirring solution is added 1N HCl aqueous solution (1 M, 1 mL) via syringe and needle before the flask is equipped with condenser and placed under nitrogen atmosphere. The reaction is heated to 75° C. with stirring overnight under nitrogen. After the reaction mixture is cooled back down to room temperature, it is suspended between layers of water and CH₂Cl₂. The aqueous layer is extracted with CH₂Cl₂ (3×) and the combined organic layer is dried with MgSO₄. The drying agent is removed by vacuum filtration and the filtrate is concentrated to dryness under reduced pressure to afford white solid thiol product (143) (42 mg, 97%) that was sufficiently pure for use in next step without further purifications. LC-MS: t_{R}=3.3 min, m/z=239.0 (ESI/[M+H]⁺).

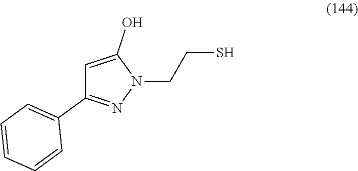 [SMILES: Oc1cc(-c2ccccc2)nn1CCS]

Synthesis of 1-(2-mercaptoethyl)-3-phenyl-1H-pyrazol-5-ol (144). The general procedure for (143) is followed using (141) (21.6 mg, 82.1 umol) to deliver thiol product (144) (17.4 mg, 96%) as colorless oil. LC-MS: 3.3 min, m/z=221.1 (ESI/[M+H]⁺).

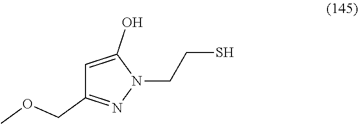

Synthesis of 1-(2-mercaptoethyl)-3-(methoxymethyl)-1H-pyrazol-5-ol (145). The general procedure for (143) is followed using (142) (54 mg, 234 umol) to deliver thiol product (145) (42 mg, 95%) as colorless oil. LC-MS: 2.7 min, m/z=189.5 (ESI/[M+H]⁺).

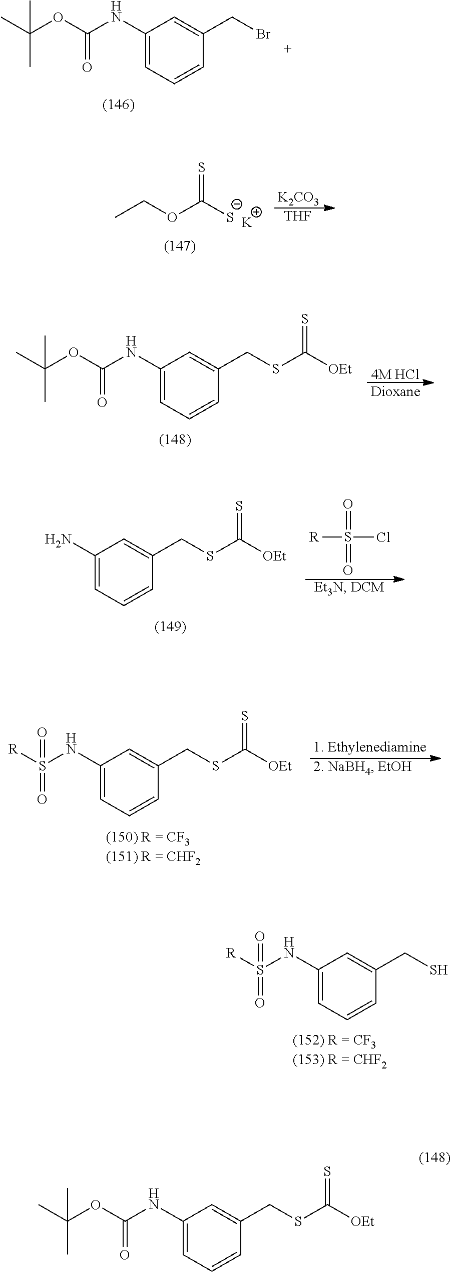

Step 1: Synthesis of O-ethyl [3-(tert-butoxycarbonylamino)phenyl] methylsulfanylmethanethioate (148). tert-butyl N-[3-(bromomethyl)phenyl]carbamate (2.0 g, 6.99 mmol) is solubilized in anhydrous THF (40 mL) and was added Potassium carbonate (2.90 g, 20.97 mmol, 3.0 equiv) and Potassium ethyl xanthate (2.80 g, 17.47 mmol, 2.5 equiv) at room temperature and stirred for 8 h. TLC/LCMS monitor suggests loss of starting material. Quenched with saturated aqueous ammonia chloride (50 mL). Extract with EtOAc (2×50 mL), and then wash water (2×50 mL) and washed organics with brine (50 mL) to give a yellow oil (2.32 g). Dry under high vac. ¹HNMR/¹³CNMR confirm desired product in acceptable purity. Carry forward without need for additional purification. LC-MS (ES+): RT 10.128 min, m/z 328.2 [M+H]⁺. ¹H NMR (400 MHz, CD₃CN) δ 7.51 (t, J=2.0 Hz, 1H), 7.32-7.23 (m, 2H), 7.04 (dt, J=7.1, 1.6 Hz, 1H), 4.68 (q, J=7.1 Hz, 2H), 4.36 (s, 2H), 1.51 (s, 9H), 1.42 (t, J=7.1 Hz, 3H).

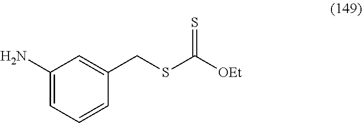 [SMILES: CCOC(=S)SCc1cccc(N)c1]

Step 2: Synthesis of O-ethyl (3-aminophenyl)methylsulfanylmethanethioate (149). To O-ethyl [3-(tert-butoxycarbonylamino)phenyl]methylsulfanylmethanethioate (2.20 g, 6.71 mmol) was added 28 mL of 4 M HCl in Dioxane at 0° C. and allowed to warm to room temperature. Stir as such for 2 hours. TLC/LCMS confirm loss of starting material and formation of desired product. Evaporate to dryness and dry under high vac overnight. (1.5 g, 98.7%). LC-MS (ES+): RT 7.763 min, m/z 228.0 [M+H]⁺. ¹H NMR (400 MHz, CD₃CN) δ 7.51 (t, J=2.0 Hz, 1H), 7.32-7.23 (m, 2H), 7.04 (dt, J=7.1, 1.6 Hz, 1H), 4.68 (q, J=7.1 Hz, 2H), 4.36 (s, 2H), 1.42 (t, J=7.1 Hz, 3H).

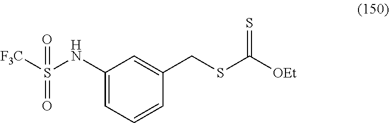 [SMILES: CCOC(=S)SCc1cccc(NS(=O)(=O)C(F)(F)F)c1]

Step 3: Synthesis of O-ethyl [3-(trifluoromethylsulfonylamino)phenyl] methylsulfanylmethanethioate (150). O-ethyl (3-aminophenyl)methylsulfanylmethanethioate (263.81 mg, 1 mmol) and Triethylamine (303.57 mg, 3.00 mmol, 418.43 uL) are combined in 3 mL of CH₂Cl₂ and stirred under N₂ in a brine ice bath. Add a 2 mL solution of CH₂Cl₂ and trifluoromethylsulfonyl trifluoromethanesulfonate (423.21 mg, 1.50 mmol, 251.91 uL) slowly and stir as such overnight allowing the yellow reaction solution to return to ambient temperature. TLC in 20% EtOAc/Hexanes confirms loss of starting material. Rotovap to dryness. Purify by flash chromatography (0-20% EtOAc/Hexanes). Pool dominant component. Dry under high vac overnight. Pale yellow solids (175.6 mg). LCMS/¹HNMR/¹³CNMR/¹⁹FNMR suggest desired product>90% purity. LC-MS (ES−): RT 10.330 min, m/z 357.9 [M−H]⁺. ¹H NMR (400 MHz, MeOD) δ 7.55 (ddd, J=7.8, 1.7, 1.0 Hz, 2H), 7.50-7.36 (m, 4H), 7.36-7.25 (m, 2H), 4.70 (s, 1H), 4.53 (q, J=7.1 Hz, 4H), 4.34 (s, 4H), 1.25 (t, J=7.1 Hz, 6H).

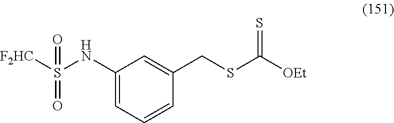 [SMILES: CCOC(=S)SCc1cccc(NS(=O)(=O)C(F)F)c1]

O-ethyl[3-(difluoromethylsulfonyl amino)phenyl]methylsulfanylmethanethioate (151). The title compound was prepared by a similar procedure as that used for (150) and purified by flash chromatography from 0-20% EtOAc/Hexanes to give (151) (236.7 mg, 63%). LC-MS (ES+): RT 3.6 min, m/z 357.4 [M+H]⁺. ¹H NMR (700 MHz, DMSO-d₆+D₂O) δ 7.32 (d, J=8.4 Hz, 2H), 7.21 (d, J=8.4 Hz, 2H), 3.82 (d, J=12.6 Hz, 2H), 3.56-3.40 (m, 1H), 3.30 (td, J=12.1, 5.0 Hz, 1H), 3.18 (td, J=12.1, 5.3 Hz, 1H), 3.09-2.80 (m, 4H), 2.75 (s, 3H), 2.04-1.98 (m, 2H), 1.68 (dtd, J=32.9, 12.1, 8.4 Hz, 2H), 1.22 (s, 9H).

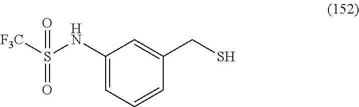 [SMILES: O=S(=O)(Nc1cccc(CS)c1)C(F)(F)F]

Step 4: Synthesis of 1,1,1-trifluoro-N-[3-(sulfanylmethyl)phenyl]methanesulfonamide (152). O-ethyl [3-(trifluoromethylsulfonylamino)phenyl]methylsulfanyl-methanethioate (175.6 mg, 488.58 umol) was dissolved in Ethylenediamine (2.25 g, 37.44 mmol, 2.5 mL) under N₂ and stirred at room temperature for 6 h. LC/MS and TLC suggest loss of starting material with dominant component ionizing as the desired product as a disulfide dimer (LC-MS (ES−) 540). The reaction was quenched by the addition of 1M H₂SO₄, and extracted with Et₂O at a pH of 11 after addition of only 10 mL 1 M H₂SO₄, and then again at a pH of 1 after addition of a total of 45 mL 1M H₂SO₄. Wash with Brine, dry over MgSO₄ and rotovap to dryness. ¹HNMR/LCMS confirmed formation of the disulfide of the desired product. Solubilize in 4 mL EtOH and heat to 70° C. Add dropwise a solution of 4 eq of sodium borohydride (73.94 mg, 1.95 mmol) in 2 mL of EtOH and continue heating for 1 h. TLC suggests loss of starting material disulfide. To the room temperature solution was added 10 mL cold water and then acidified using 1N HCl (2.0 mL) from pH 11 to ˜pH 1. MeOH evaporated from the milky white solution and extracted with EtOAc (3×25 mL). Dry over MgSO₄ and evaporate to dryness to give a yellow solid (88.0 mg, 66.3%) in >90% purity. LC-MS (ES−): RT 9.178 min, m/z 270.28 [M−H]⁺. ¹H NMR (400 MHz, CDCl₃) δ 7.26 (d, J=9.0 Hz, 6H), 7.21-7.16 (m, 1H) 7.12 (d, J=8.1 Hz, 6H), 7.05 (s, 3H), 3.92 (q, J=7.2 Hz, 5H), 3.49 (s, 6H), 1.21-1.06 (m, 11H).

Synthesis of 1,1-difluoro-N-[3-(sulfanylmethyl)phenyl]methanesulfonamide (153) was performed as above for compound (152) from starting material (151).

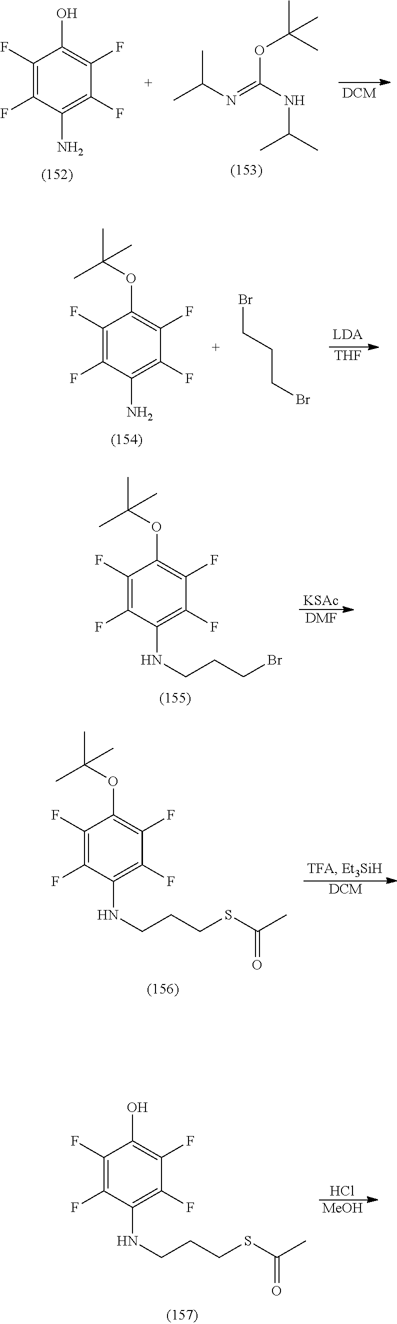

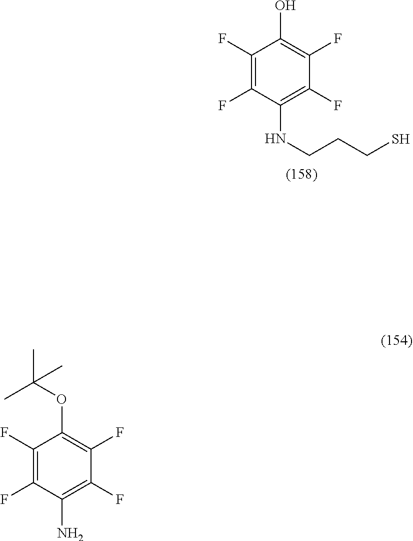 [SMILES: Oc1c(F)c(F)c(NCCCS)c(F)c1F ; CC(C)(C)Oc1c(F)c(F)c(N)c(F)c1F]

Step 1: Synthesis of 4-tert-butoxy-2,3,5,6-tetrafluoroaniline (154). To a stirred solution of 4-amino-2,3,5,6-tetrafluorophenol (152) (0.445 g, 2.46 mmol) in CH₂Cl₂ at room temperature under N₂ was added 1 molar equivalent of 2-tert-butyl-1,3-diisopropylisourea (2.63 g, 13.13 mmol, 2.95 mL) every 2 hours until the total 5 equiv was added and the reaction is allowed to stir overnight. LC-MS monitor suggests no starting material retention. TLC in 20% EtOAc/Hexanes suggests complete conversion from starting material. One component of interest ˜0.5 rF. Rotovap CH₂Cl₂ and triturate in Hexanes, overnight Filter. Set insoluble diisopropylurea byproduct aside. Rotovap organics to give a dark brown oil that is dried under high vac to give (0.51 g, 87.5%) desired product as a dark brown gum in >90% purity. LC-MS (ES+): RT 8.856 min, m/z 238.0 [M−H]⁺. ¹H NMR (400 MHz, CD₃CN) δ 4.27 (s, 1H), 1.25-1.18 (m, 6H).

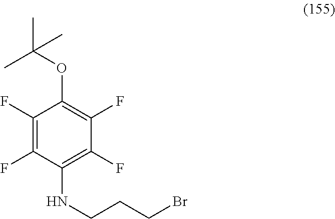 [SMILES: CC(C)(C)Oc1c(F)c(F)c(NCCCBr)c(F)c1F]

Step 2: Synthesis of N-(3-bromopropyl)-4-tert-butoxy-2,3,5,6-tetrafluoroaniline (155). To a 20 mL sealed round bottom flushed with nitrogen and equipped with a magnetic stir bar containing 4-tert-butoxy-2,3,5,6-tetrafluoroaniline (154) (0.2586 g, 1.09 mmol) was dissolved with THF (2 mL). The solution was stirred under nitrogen and cooled down to −78° C. over 15 min. To this solution at −78° C. was then added LDA (1 M, 1.31 mL) as a solution in THE and stirred for 1 hour at the same temperature. After 1 h, a solution of 1,3-dibromopropane (662.80 mg, 3.28 mmol, 334.75 uL) was added to the mixture at −78° C. and warmed up to room temperature with stirring over 1 h. Stirring at room temperature overnight. TLC/LCMS suggests ˜80% conversion. Concentrated to dryness. (0.5219 g) Dark brown oil/gum. Column chromatography 0-20% EtOac/Hexanes eluted out product in >90% purity (239.7 mg, 61.4%). LC-MS (ES+): RT 10.318 min, m/z 359.0 [M−H]⁺. ¹H NMR (400 MHz, CDCl₃) δ 3.57 (d, J=5.7 Hz, 1H), 3.47-3.36 (m, 4H), 2.06 (p, J=6.5 Hz, 2H), 1.29 (t, J=1.2 Hz, 9H).

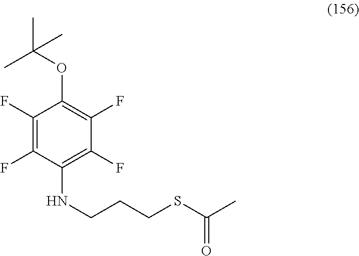 [SMILES: CC(=O)SCCCNc1c(F)c(F)c(OC(C)(C)C)c(F)c1F]

Step 3: Synthesis of S-[3-(4-tert-butoxy-2,3,5,6-tetrafluoroanilino)propyl] ethanethioate (156) To a stirred solution of N-(3-bromopropyl)-4-tert-butoxy-2,3,5,6-tetrafluoroaniline (155) (0.2397 g, 669.24 umol) in DMF (3.6 mL) was added 1 eq of potassium thioacetate (76.43 mg, 669.24 umol) and stirred at room temperature overnight. Starting material was consumed as observed by TLC (10% EtOAc in hexanes). The reaction mixture was concentrated to dryness. Crude material was purified using 0-20% EtOAc in hexanes gradient. (177.8 mg, 75.2%). LC-MS (ES+): RT 10.385, m/z 354.0 [M−H]⁺. ¹H NMR (400 MHz, MeOD) δ 3.23 (tt, J=6.8, 1.5 Hz, 2H), 2.83 (t, J=7.1 Hz, 2H), 2.20 (s, 3H), 1.71 (p, J=6.9 Hz, 2H), 1.23 (t, J=1.2 Hz, 9H).

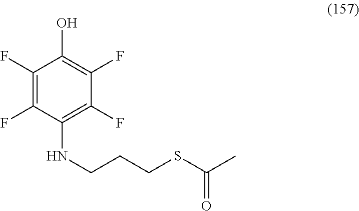 [SMILES: CC(=O)SCCCNc1c(F)c(F)c(O)c(F)c1F]

Step 4: Synthesis of S-[3-(2,3,5,6-tetrafluoro-4-hydroxy-anilino)propyl] ethanethioate (157) To a 100 mL RBF containing the S-[3-(4-tert-butoxy-2,3,5,6-tetrafluoro-anilino)propyl] ethanethioate (156) (177.8 mg, 503.15 umol) was dissolved with CH₂Cl₂ (5 mL). To this stirring mixture was added triethylsilane (70.62 mg, 607.31 umol, 97 uL) followed by TFA (1.27 g, 11.11 mmol, 850 uL) before stirring at room temperature under N₂. LCMS of white slurry confirms that the starting material has been consumed and formation of desired product after 4 hours. The reaction is then concentrated to dryness and purified by flash column chromatography using a gradient of 0-20% EtOAc in hexanes. Desired product is obtained as white solids in >90% purity. (77.5 mg, 51.8%) LC-MS (ES+): RT 7.928, m/z 298.0 [M−H]⁺. ¹H NMR (400 MHz, CD₃CN) δ 3.26 (tt, J=6.8, 1.4 Hz, 2H), 2.93 (t, J=7.1 Hz, 2H), 2.32 (s, 3H), 1.79 (p, J=6.9 Hz, 2H).

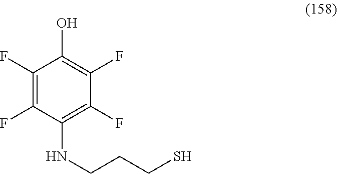 [SMILES: Oc1c(F)c(F)c(NCCCS)c(F)c1F]

Step 5: Synthesis of 2,3,5,6-tetrafluoro-4-(3-sulfanylpropylamino)phenol (158). After heating S-[3-(2,3,5,6-tetrafluoro-4-hydroxy-anilino)propyl]ethanethioate (77.5 mg, 260.71 umol) in a solution of 1M aqueous HCl (6 mL, 6 mmol) and MeOH (6 mL) to 90° C. for 4 h, loss of starting material observed by LCMS. The reaction was cooled down to room temperature before diluted with CH₂Cl₂ and water. The aqueous layer was extracted with CH₂Cl₂ (3×) and the combined organic layer was dried with magnesium sulfate before filtering the drying reagent and concentrated to dryness to give the desired product (65 mg, 97.7%) as a colorless oil in >90% purity. LC-MS (ES+): RT 6.223, m/z 256.0 [M−H]⁺. ¹H NMR (400 MHz, CD₃CN) δ 3.33 (ddt, J=9.4, 5.0, 1.8 Hz, 2H), 2.59 (q, J=7.3 Hz, 2H), 1.91-1.76 (m, 2H).

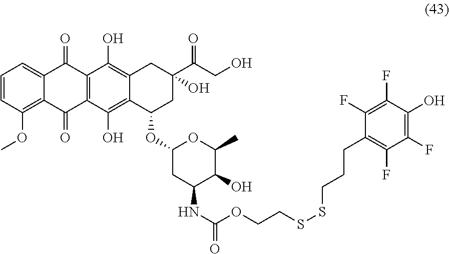 [SMILES: COc1cccc2c1C(=O)c1c(O)c3c(c(O)c1C2=O)C[C@@](O)(C(=O)CO)C[C@@H]3O[C@H]1C[C@H](NC(=O)OCCSSCCCc2c(F)c(F)c(O)c(F)c2F)[C@H](O)[C@H](C)O1]

Synthesis of YU244206 (43). To a nitrogen flushed 10 mL RBF equipped with magnetic stir bar was added Dox-SS-Py ((123), 52 mg, 68.7 umol) followed by 2,3,5,6-tetrafluoro-4-(3-sulfanylpropyl)phenol (132) (16.5 mg, 68.7 umol, 1.0 equiv) before dissolving with CH₂Cl₂ (2 mL) and DMF (2 mL). The mixture was stirred away from light by wrapping the flask in aluminum foil and at room temperature for 24 h where the reaction is monitored by LC-MS. After (123) has been consumed, the reaction mixture is then concentrated down before loaded onto a SNAP Ultra 10 g silica gel column. The product is eluted with 0-10% MeOH in CH₂Cl₂ gradient and the solvents are removed under reduced pressure (with toluene added) to deliver (43) (52.5 mg, 86%) as bright red solid. ¹⁹F NMR (376 MHz, Chloroform-d) δ −146.59 (dd, J=21.7, 8.3 Hz), −163.15 (dd, J=21.7, 8.1 Hz); LC-MS: t_{R}=3.7 min, m/z=487.9 (ESI/[M−H]⁻).

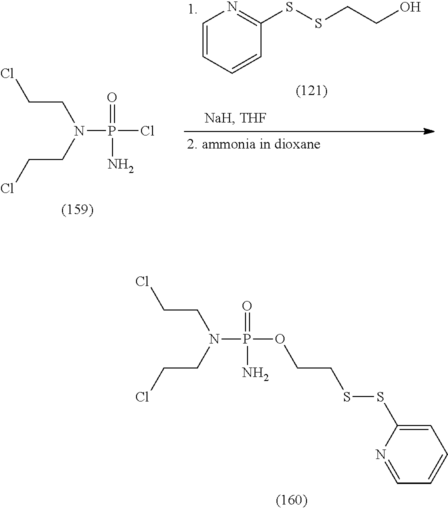 [SMILES: NP(=O)(Cl)N(CCCl)CCCl ; OCCSSc1ccccn1 ; "(121)" ; "(159)" ; NP(=O)(OCCSSc1ccccn1)N(CCCl)CCCl ; "(160)"]

Compound (121) (200 mg, 1.07 mmol) in THF (3 mL) was added NaH (60% in mineral oil, 51 mg, 1.30 mmol) at 0° C. The suspension stirred for 30 min at 0° C. and added (159) (276 mg, 1.06 mmol) in 2 mL THF. The reaction mixture warmed to room temperature and stirring was continued for 4 h at room temperature. The solvent was removed by rotary evaporator, the crude oil was dissolved in dioxane (2 mL) and added the excess of ammonia in dioxane (0.5 M) at room temperature. The stirring was continued for overnight, the dioxane was evaporated and purified by silica gel column chromatography (CH₂Cl₂:MeOH=9:1) to provide compound (160) (29 mg, 7% yield). ¹H NMR (500 MHz, Chloroform-d) δ 8.46-8.40 (m, 1H), 7.69-7.58 (m, 2H), 7.07 (ddd, J=6.7, 4.8, 2.0 Hz, 1H), 4.30-4.07 (m, 2H), 3.58 (t, J=7.1 Hz, 4H), 3.46-3.35 (m, 4H), 3.03 (t, J=6.3 Hz, 2H); ¹³C NMR (126 MHz, cdcl₃) δ 159.34, 149.69, 137.17, 121.02, 120.08, 63.21, 63.17, 49.22, 49.18, 42.51, 39.06, 39.01; ³¹P NMR (202 MHz, cdcl₃) δ 16.16; LC-MS: (M+H)⁺=390.31 (experimental), 390.00 (calculated).

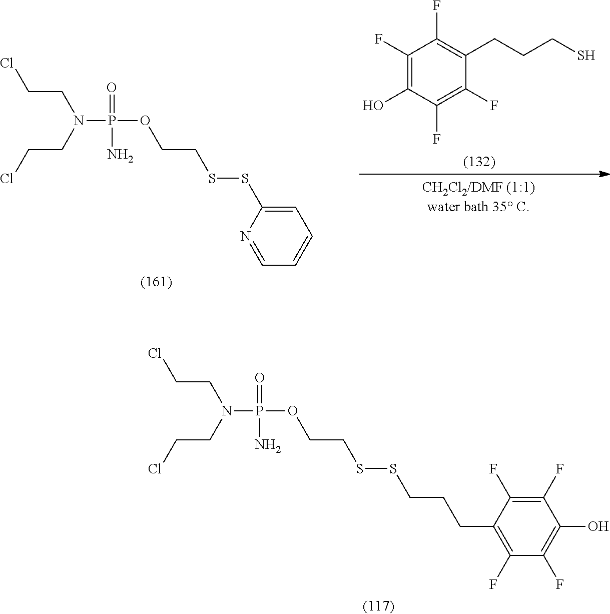

Compound (161) (25 mg, 0.06 mmol) and compound (132) (31 mg, 0.13 mmol) were dissolved in CH₂Cl₂/DMF (1:1) 1 mL. The reaction was stirred at 35° C. water bath for 24 hours. The solvent was removed under vacuum and purified by silica gel column chromatography (Hexanes:EtOAC=2:3) to provide compound (117) (13 mg, 42% yield). ¹H NMR (500 MHz, Chloroform-d) δ 4.29-4.07 (m, 2H), 3.72-3.57 (m, 4H), 3.54-3.38 (m, 4H), 2.90 (td, J=6.6, 2.2 Hz, 2H), 2.78-2.63 (m, 4H), 1.95 (t, J=7.4 Hz, 2H); ³¹P NMR (202 MHz, Chloroform-d) δ 15.77; ¹⁹F NMR (471 MHz, Chloroform-d) 6-146.84 (dd, J=21.6, 8.2 Hz, 2F), −161.33-−164.94 (m, 2F); HRMS: (M+H)⁺=519.0028 (experimental), 519.0117 (calculated).

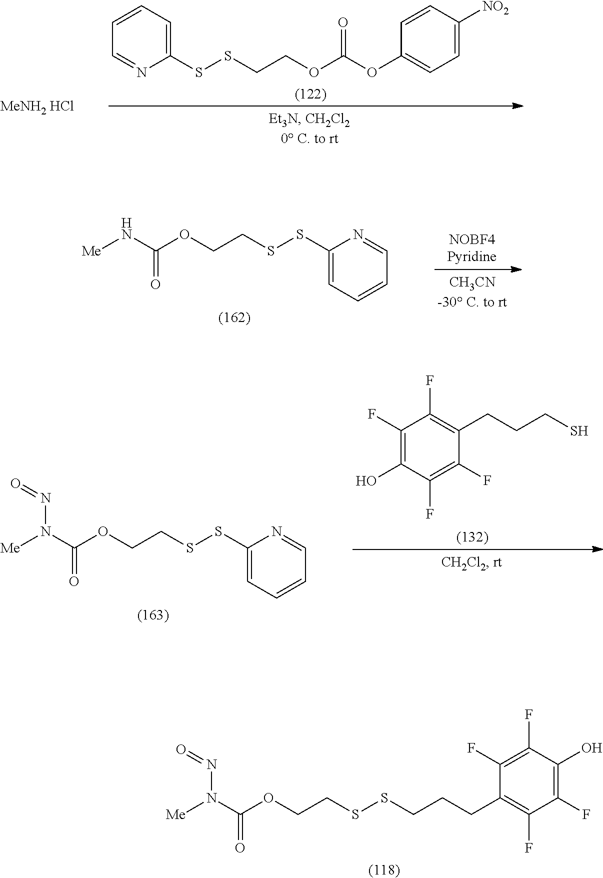

Methylamine hydrochloride (200 mg, 3 mmol) and carbonate (122) (500 mg, 1.42 mmol) in CH₂Cl₂ (10 mL) was added Et₃N (0.7 mmol, 5 mmol) at 0° C. The reaction mixture warmed to room temperature and stirred for 12 h. The solvent was evaporated purified through silica gel to provide product (162). Compound (162) (87 mg, 0.36 mmol) dissolved in acetonitrile (3 mL) were added nitroso tetrafluoroborate (96 mg, 0.82 mmol) and pyridine (57 μL, 0.712 mmol) at −30° C. under nitrogen. The reaction mixture stirred for 2 h at same temperature. The solvent was evaporated and purified by silica gel column chromatography to provide (163) (LCMS: calculated (M+H)⁺ 274.03, experimental 274.57). Compound (163) (11 mg, 0.04 mmol) in CH₂Cl₂ (2 mL) was added (132) under nitrogen at room temperature. The reaction mixture was stirred for 24 h at room temperature and the crude material was purified on silica gel column chromatography to provide (118). ¹H NMR (400 MHz, Chloroform-d) δ 4.72 (t, J=6.7 Hz, 2H), 3.16 (s, 3H), 3.07 (t, J=6.8 Hz, 2H), 2.87-2.61 (m, 4H), 1.97 (p, J=7.4 Hz, 2H); ¹⁹F NMR (376 MHz, Chloroform-d) δ −146.01-−146.16 (m, 2F), −163.61-−163.73 (m, 2F). LCMS: (M−H)⁺=401.0 (calculated), 400.9 (experimental).

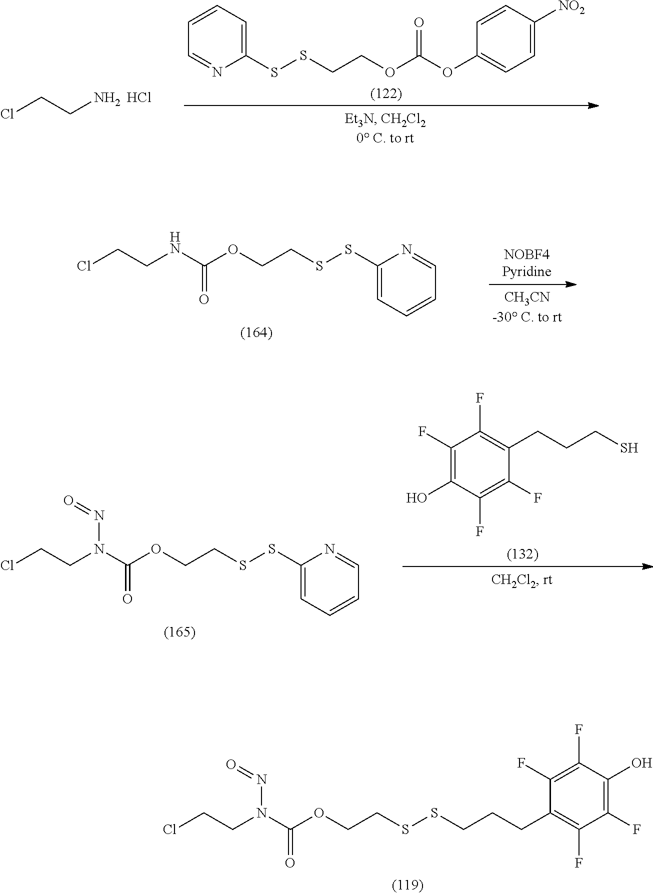

Synthesis of (164) is similar to that of (162). Characterization data of (164): ¹H NMR (400 MHz, Chloroform-d) δ 8.35 (dt, J=4.8, 1.3 Hz, 1H), 7.70-7.48 (m, 2H), 6.99 (ddd, J=7.2, 4.9, 1.3 Hz, 1H), 5.57 (t, J=5.9 Hz, 1H), 4.21 (d, J=6.4 Hz, 2H), 3.48 (t, J=5.9 Hz, 2H), 3.38 (q, J=5.9 Hz, 2H), 2.92 (t, J=6.4 Hz, 2H); ¹³C NMR (101 MHz, cdcl₃) δ 159.61, 155.96, 149.54, 137.09, 120.81, 119.70, 62.68, 43.71, 42.73, 37.71. LCMS: calculated for C₁₀H₁₄ClN₂O₂S₂ 293.02, found 293.03.

Synthesis of (165) is similar to that of (163). Characterization data of (165): ¹H NMR (500 MHz, Chloroform-d) δ 8.47 (d, J=4.8 Hz, 1H), 7.76-7.57 (m, 2H), 7.10 (dd, J=7.1, 4.9 Hz, 1H), 4.75 (t, J=6.5 Hz, 2H), 4.07 (t, J=6.7 Hz, 2H), 3.45 (t, J=6.6 Hz, 2H), 3.22 (t, J=6.5 Hz, 2H); ¹³C NMR (126 MHz, cdcl₃) δ 159.10, 153.40, 149.80, 137.10, 121.08, 120.11, 65.86, 41.36, 38.82, 36.82. LCMS: calculated for C₁₀H₁₃ClN₃O₃S₂ 322.01, found 322.06.

Synthesis of (119) is similar to that of (118): Characterization data of S14: ¹H NMR (500 MHz, Chloroform-d) δ 4.75 (t, J=6.7 Hz, 2H), 4.10 (t, J=6.6 Hz, 2H), 3.47 (t, J=6.6 Hz, 2H), 3.07 (t, J=6.7 Hz, 2H), 2.80-2.70 (m, 4H), 2.00-1.93 (m, 2H). ¹⁹F NMR (470 MHz, Chloroform-d) 6-146.18-−146.27 (m, 2F), −160.32-−165.35 (m, 2F). LCMS: calculated for C₁₄H₁₆ClF₄N₂O₄S₂(M+H)⁺449.00, found 448.91.

Compounds of Claim 32 are generally synthesized similar to published synthetic routes for other derivatives of the pharmacophores, for example using the synthetic approaches in Zhang, et al. Design, synthesis, SAR discussion, in vitro and in vivo evaluation of novel selective EGFR modulator to inhibit L858R/T790M double mutants. European Journal of Medicinal Chemistry 135 (2017) 12-23, which is incorporated herein by reference.

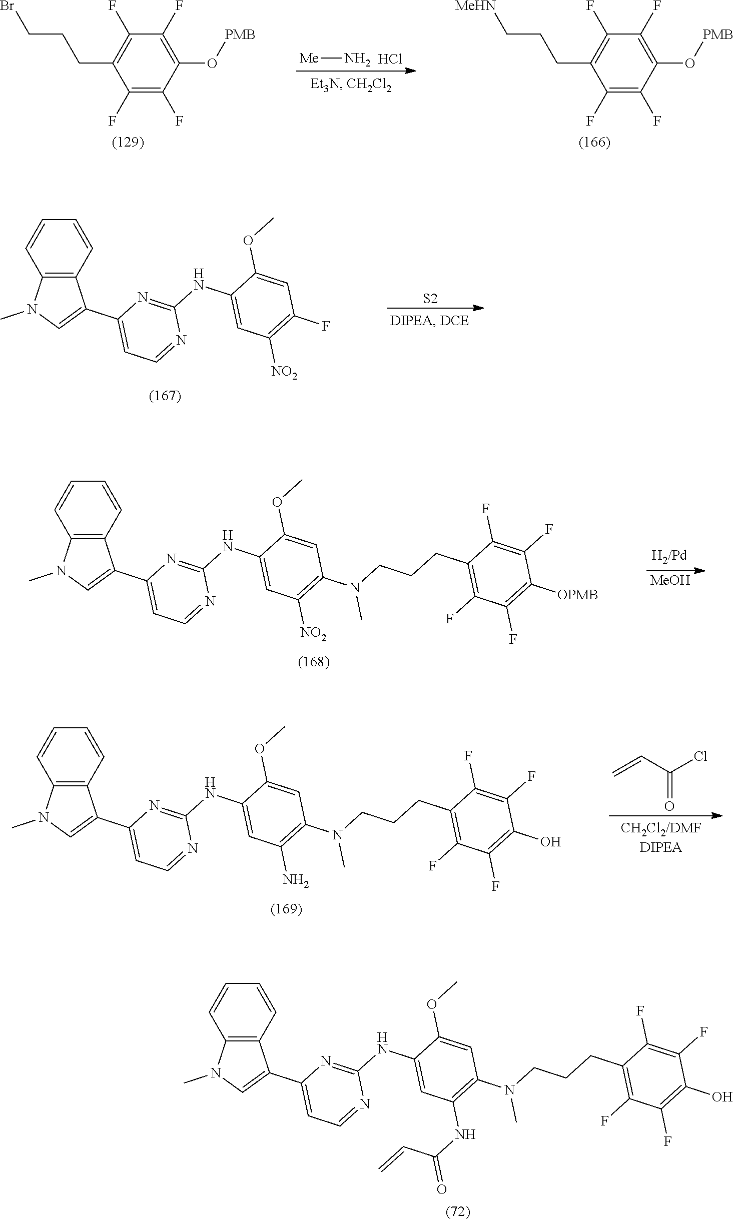

Compound (72) was synthesized as show above. Characterization data of Compound (72): ¹H NMR (400 MHz, DMSO-d₆) δ 9.12 (s, 1H), 8.78 (s, 1H), 8.53 (s, 1H), 8.40-8.07 (m, 2H), 7.85 (s, 1H), 7.48 (d, J=8.1 Hz, 1H), 7.32-7.02 (m, 3H), 6.86 (s, 1H), 6.58 (dd, J=17.0, 10.2 Hz, 1H), 6.17 (d, J=17.0 Hz, 1H), 5.66 (d, J=10.3 Hz, 1H), 3.86 (s, 3H), 3.81 (s, 3H), 2.82 (t, J=7.6 Hz, 2H), 2.70-2.54 (m, 5H), 1.69 (t, J=7.6 Hz, 2H). LCMS: calculated for C₃₃H₃₁F₄N₆O₃ [M+H]⁺ 635.2, observed 635.6.

CDK family kinase inhibitors are generally synthesized according to published synthetic routes for other derivatives of the pharmacophores, for example using the synthetic approaches in Tadesse, et al. Highly Potent, Selective, and Orally Bioavailable 4-Thiazol-N-(pyridin-2-yl)pyrimidin-2-amine Cyclin-Dependent Kinases 4 and 6 Inhibitors as Anticancer Drug Candidates: Design, Synthesis, and Evaluation. Journal of Medicinal Chemistry (2017) 60, 1892-1915, which is incorporated herein by reference.

VEGFR and related kinase inhibitors are generally synthesized similar to published synthetic routes for other derivatives of the pharmacophores, for example using the synthetic approaches in Jin, et al. Synthesis and Biological Evaluation of 3-Substituted-indolin-2-one Derivatives Containing Chloropyrrole Moieties. Molecules (2011) 16, 9368-9385, which is incorporated herein by reference.

Anaplastic lymphoma kinase (ALK) inhibitors are generally synthesized similar to published synthetic routes for other derivatives of the pharmacophores, for example using the synthetic approaches in Marsilje, et al. Synthesis, Structure-Activity Relationships, and in Vivo Efficacy of the Novel Potent and Selective Anaplastic Lymphoma Kinase (ALK) Inhibitor 5-Chloro-N2-(2-isopropoxy-5-methyl-4-(piperidin-4-yl)phenyl)-N4-(2-(isopropylsulfonyl)phenyl)pyrimidine-2,4-diamine (LDK378) Currently in Phase 1 and Phase 2 Clinical Trials. Journal of Medicinal Chemistry (2013) 56(14), 5675-5690, which is incorporated herein by reference.

PARP inhibitors are generally synthesized similar to published synthetic routes for other derivatives of the pharmacophores, for example using the synthetic approaches in Wang, et al. Design, Synthesis, and Biological Evaluation of Novel PARP-1 Inhibitors Based on a 1H-Thieno[3,4-d] Imidazole-4-Carboxamide Scaffold. Molecules (2016) 21, 772, which is incorporated herein by reference.

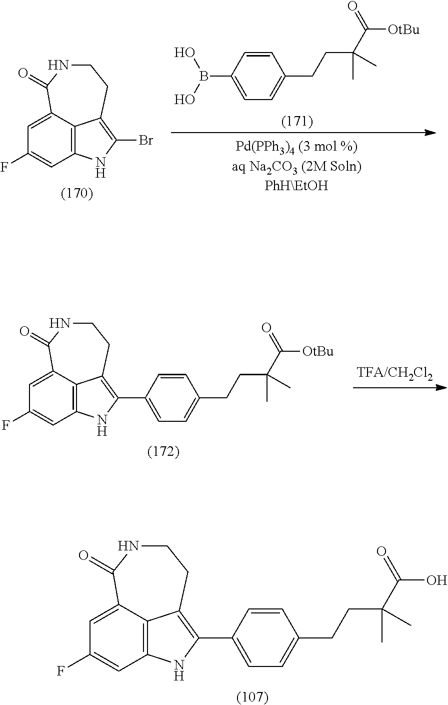

Synthesis of Compound (107). Heteroaryl bromide (170) (50 mg, 0.18 mmol) and Pd(PPh₃)₄ (6 mg, 0.05 mmol) in benzene (3.6 mL) were added 2 M aq. Na₂CO₃ (1.8 mL), boronic acid (171) (63 mg, 0.22 mmol), and ethanol (0.9 mL) under nitrogen at room temperature. The reaction mixture was then heated to 80° C. and stirred for overnight. The cooled mixture dissolved in water and washed with EtOAc (2×5 mL), the combined organic layers were dried over anhydrous sodium bicarbonate. The concerted crude material was purified by column chromatography (Hexanes:EtOAc) to provide product (172). ¹H NMR (500 MHz, Chloroform-d) δ 10.03 (s, 1H), 7.71-7.65 (m, 1H), 7.55-7.51 (m, 1H), 7.49-7.37 (m, 3H), 7.24-7.17 (m, 2H), 3.52 (d, J=7.2 Hz, 2H), 3.19-3.03 (m, 2H), 3.00-2.92 (m, 1H), 2.64-2.44 (m, 2H), 1.77 (dd, J=11.4, 6.1 Hz, 2H), 1.48 (s, 9H), 1.19 (s, 6H). Compound (172) was dissolved in CH₂Cl₂:TFA (2:1) and stirred for 1 h at room temperature. The solvent was removed under rotary evaporator and purified by column chromatography (CH₂Cl₂:MeOH) to provide the acid (107). ¹H NMR (400 MHz, Methanol-d₄) δ 11.13 (s, 1H), 7.68-7.44 (m, 3H), 7.32 (t, J=8.9 Hz, 3H), 5.51 (s, 1H), 3.71-3.44 (m, 2H), 3.13 (dd, J=6.2, 3.4 Hz, 2H), 2.81-2.49 (m, 2H), 2.02-1.78 (m, 2H), 1.28 (d, J=2.7 Hz, 6H); ¹³C NMR (101 MHz, cd₃od) δ 180.14, 171.23, 160.19, 157.85, 142.29, 137.12, 137.00, 136.16, 129.47, 128.35, 127.73, 124.22, 124.12, 123.64, 111.08, 109.83, 109.57, 100.94, 100.68, 42.50, 42.41, 41.77, 31.03, 28.56, 24.28. ¹⁹F NMR (376 MHz, cd₃od) δ −123.56. LC-MS: calculated for C₂₃H₂₄FN₂O₃[M+H]⁺ 395.17, found 395.12.

Estrogen receptor modulators are generally synthesized similar to published synthetic routes for other derivatives of the pharmacophores, for example using the synthetic approaches in Shoda, et al. Synthesis and evaluation of raloxifene derivatives as a selective estrogen receptor down-regulator. Bioorganic Medicinal Chemistry (2016) 24(13), 2914-2919, which is incorporated herein by reference.

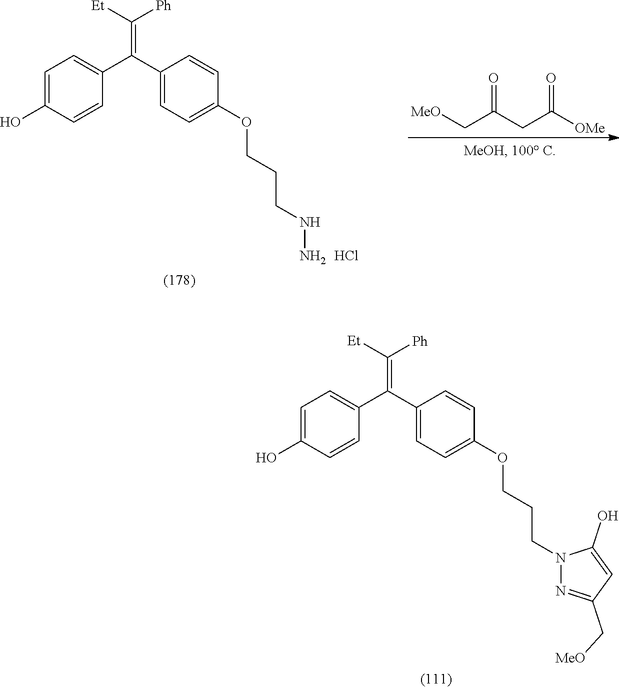 [SMILES: CC/C(=C(\c1ccc(O)cc1)c1ccc(OCCCNN)cc1)c1ccccc1 ; Cl ; COCC(=O)CC(=O)OC ; "(178)" ; CC/C(=C(\c1ccc(O)cc1)c1ccc(OCCCn2nc(COC)cc2O)cc1)c1ccccc1 ; "(111)"]

Synthesis of Compound (111). In a sealed tube tamoxifen hydrazine (178) (21 mg, 0.05 mmol) in 2 mL methanol was added methyl 4-methoxyacetoacetate (15 mg, 0.1 mmol) under nitrogen. The reaction mixture than heated to 100° C. for overnight and cooled to room temperature. The solvent was evaporated and purified by silica gel column chromatography (CH₂Cl₂:MeOH) to provide the tautomeric product (111). ¹H NMR (600 MHz, Methanol-d₄) δ 7.17-7.01 (m, 7H), 6.99 (d, J=8.1 Hz, 1H), 6.85 (d, J=8.1 Hz, 1H), 6.74 (d, J=8.0 Hz, 1H), 6.71 (d, J=8.4 Hz, 1H), 6.63 (d, J=8.3 Hz, 1H), 6.48 (d, J=8.3 Hz, 1H), 6.38 (d, J=8.1 Hz, 1H), 4.27 (s, 1H), 4.23 (s, 1H), 4.09 (t, J=6.9 Hz, 1H), 4.00 (t, J=6.9 Hz, 1H), 3.95 (t, J=6.0 Hz, 1H), 3.79 (t, J=6.1 Hz, 1H), 3.32-3.25 (m, 3H), 2.45 (dq, J=14.8, 7.7 Hz, 2H), 2.19 (p, J=6.6 Hz, 1H), 2.08 (p, J=6.6 Hz, 1H), 0.88 (t, J=7.4 Hz, 3H); ¹³C NMR (151 MHz, Methanol-d₄) δ 207.53, 157.62, 156.78, 155.83, 154.94, 147.59, 147.51, 142.68, 140.48, 140.32, 138.32, 136.29, 135.91, 134.94, 134.56, 131.61, 131.56, 131.53, 131.30, 131.11, 130.15, 130.09, 129.84, 129.46, 127.51, 127.44, 127.42, 125.53, 125.50, 114.43, 114.41, 113.69, 113.66, 113.56, 112.90, 67.50, 64.61, 64.41, 56.87, 56.85, 48.43, 42.31, 28.90, 28.82, 28.49, 28.43, 12.54, 12.52. LC-MS calculated for C₃₀H₃₃N₂O₄ [M+H]⁺ 485.24, observed 485.36.

### In Vitro Studies

Doxorubicin is widely used in anticancer chemotherapy. However, it produces a high incidence of side-effects, including lifetime dose-limiting irreversible cardiotoxicity. These side-effects have been to some extent attributed to doxorubicin's weakly-basic nature, which contributes to poor cell permeability in acidic tumors as well as being correlated with its cardiotoxicity. Because of the severity of these side-effects, formulations and derivatizations that improve drug tolerance have been extensively pursued. Liposomal forms of doxorubicin showed decreased cardiotoxicity. These formulations slightly improve tumor specificity through the enhanced-permeation/retention (EPR) effect, but primarily act as slow-release encapsulations, lowering and broadening the blood plasma concentration curve following administration. However, while improved in tolerance, the encapsulated formulation does not address its weakly-basic nature, so its uptake bias and dose-limiting cardiotoxicity persist. Anthracycline cardiotoxicity is thought to come at least in part from active uptake by cardiomyocytes, due to recognition of the amine-bearing sugar functionality in the anthracycline structure. In certain embodiments, the prodrugs of the invention decrease cardiac uptake as evidenced by the published protective effects of amidization of the amine.

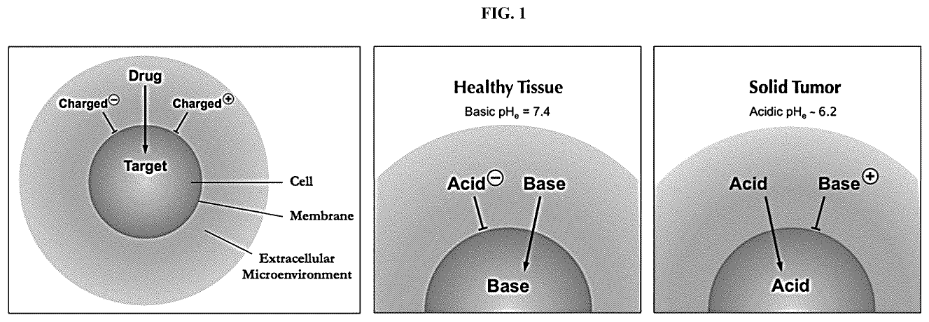

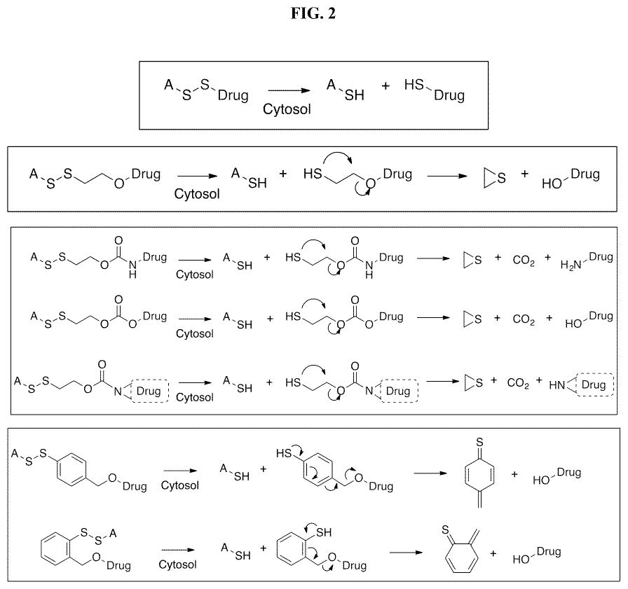

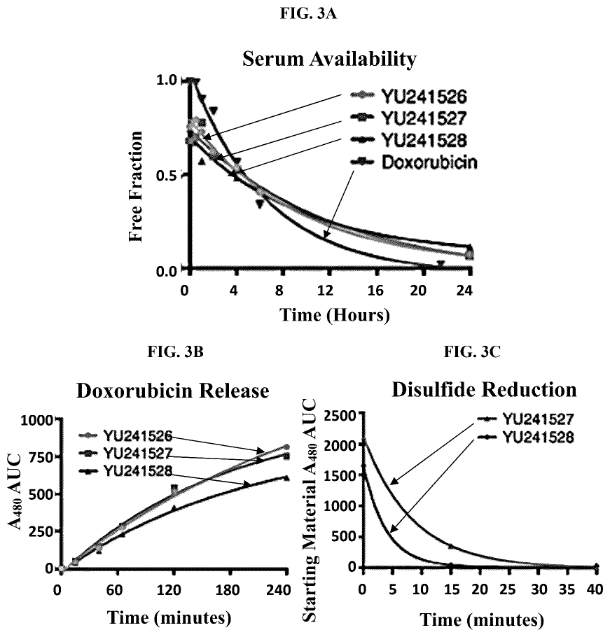

Doxorubicin uptake in tumors occurs by a process of passive diffusion through the cell membrane, based on a concentration gradient from the blood or extracellular fluid into the cytosol. The pH of the extracellular environment influences the potential for a weakly ionic drug, such as doxorubicin, to permeate a cell (FIG. 1). The Henderson-Hasselbalch equation allows for calculating the neutral fraction of an ionizable drug at healthy tissue vs. tumor extracellular pH, and thus predicting the associated cell-permeable fractions. Doxorubicin's amine group has a basic pK_{A} of ˜8, and so the fraction predicted to be non-ionized and membrane permeable is ˜7-fold greater at a pH of 7.4 than at 6.5. The carboxyl group of YU241528 has an acidic pK_{A} of ˜4.4, so the predicted membrane permeable fraction is ˜8-fold less at a pH of 7.4 than at 6.5. This accurately predicts the orientation, if not the scale of doxorubicin's uptake bias, in vitro, towards cells in healthy tissues, and predicts that YU241528 is favored to a similar degree to permeate cells in tumors rather than healthy tissues.

### Serum Stability and Reduction of Prodrugs

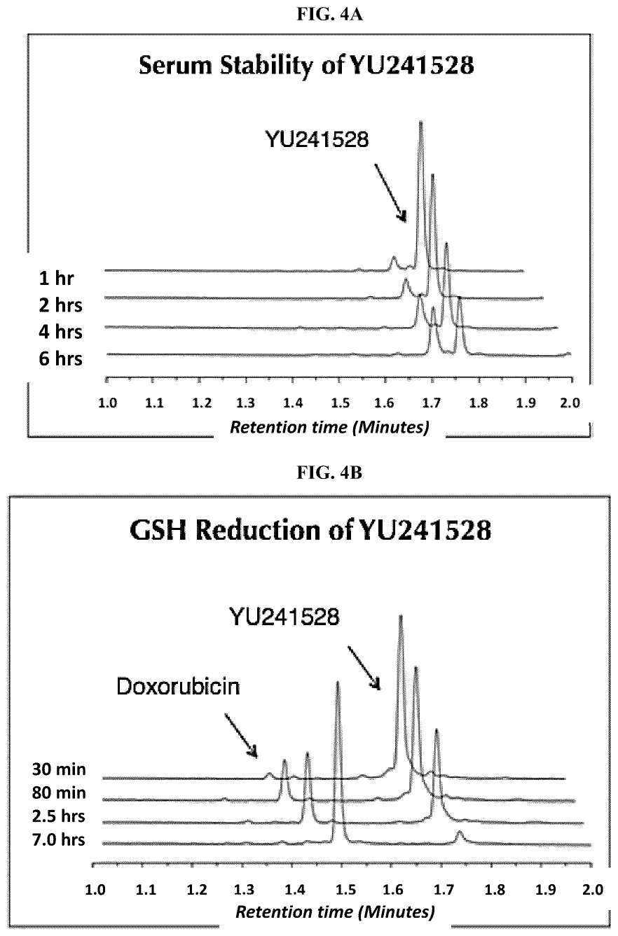

The prodrug strategies of this invention are thought to impart their benefits during the distribution and cell uptake steps of anticancer therapy. In certain embodiments, a prodrug is stable in the blood for a period sufficient to allow for cell uptake and systemic clearance. To test this, 50 μM YU241528 in a solution of PBS, pH 7.4, containing 20% mouse serum was incubated at 37° C. At regular intervals, aliquots were taken and mixed with 2 volumes of ethanol to precipitate serum proteins, then centrifuged. Supernatants were analyzed by LCMS. Doxorubicin is a chromophore with an absorbance peak of around 490 nm (EC₄₉₀-11300 M⁻¹ cm⁻¹), which is also observable for prodrugs of doxorubicin. Chromatograms of 490 nm absorbance were compared over the range of incubation times to track changes in the retention of products containing doxorubicin. The predominant peak was identified as YU241528 by mass spectrometry. While the free fraction of YU241528 diminished over time, likely due to protein binding, and some interaction with serum was observed with a t_{1/2} greater than 6 hours, no free doxorubicin was detected over the 6 hours of incubation (FIG. 4A).

In certain embodiments, the prodrug releases the active form of the drug once inside a cancer cell. Once inside the cell, drug release can be triggered by reduction of the disulfide bond in the linker. To evaluate the reductive activation of drug release, 50 μM YU241528 in a solution of PBS, pH 7.4, containing 5 mM GSH (a typical intracellular concentration of the predominant biological reducing agent) was incubated at 37° C. Aliquots were taken at regular intervals and analyzed by LCMS. Chromatograms of 490 nm absorbance were compared over the range of incubation times to track doxorubicin release. Reduction of the disulfide, detected by peak shift and change in molecular mass, was detected with a t_{1/2} of ˜2.7 minutes. The prodrug and its reduced intermediate were converted to doxorubicin with a t_{1/2} of ˜3 hours (FIG. 4B).

Doxorubicin exhibits rapid distribution from the blood into tissues, with a t_{1/2} on the order of ˜5 minutes following intravenous injection, and is eliminated by the hepatobiliary route, as well as by catabolism, with a t_{1/2} of ˜20 to 48 hours. YU241528 is stable in serum, resisting release of the active doxorubicin structure and is bioavailable past the 6 hours measured in vitro, however it interacts with serum to produce an unidentified metabolite with a t_{1/2} of >8 hours. Given that the drug's distribution and clearance can be expected to be substantially complete by about 6 hours after administration, in certain embodiments the peak intracellular dose is achieved long before this process might interfere to a significant degree, even assuming the altered metabolite is indeed compromised in its activity. In adult female balb/c mice, YU241528 injected into the tail vein remains at detectable levels in the blood at 6 hours post injection, with a non-compartmental half-life of about 1 hour, then is undetectable at 24 hours post injection. In certain embodiments, the serum stability of YU241528 allows for effective therapy. Without wishing to be limited by any theory, because there is no evidence of doxorubicin release over that same time, the observed degradation (or augmentation) of the prodrug in serum does not contribute to side effects. In certain embodiments, the prodrug undergoes a different elimination path, as hepatobiliary elimination of weakly-ionic organic substances is sensitive to charge, size, and lipophilicity.

The cytotoxic effects of doxorubicin at therapeutic doses occur in the nucleus, where doxorubicin acts primarily as a topoisomerase-II inhibitor. This effect is observed after a significant delay following uptake of the drug into the cell. YU241528 was processed in intracellular conditions (5 mM GSH) to restore the active doxorubicin structure with a t_{1/2} of ˜3 hours. Since the appearance of cytotoxic response to doxorubicin occurs on the order of hours to days after exposure, this timescale allows for near-maximal effect of the delivered dose. Without wishing to be limited by any theory, mass spectroscopic evidence suggests that the rate-limiting step of doxorubicin release from the prodrug is the elimination of the 2-thioethylcarbamate linker from doxorubicin's amine group. The reductive strength within cancer cells is significantly greater than in average healthy cells, and this step can occur more rapidly in cancerous cells than in healthy cells. The more restrictive condition of 5 mM GSH was used as a proof-of-principle. Cancer's greater capacity to reductively trigger drug release and activation may further bias the tumor-specific activity of the prodrug. This may be of particular consequence to the side-effect of cardiotoxicity, as cardiomyocytes have lower than average intracellular GSH. If reductive activation is necessary for the prodrug to become toxic, the combination of the limited uptake of the prodrug into cardiac cells and the limited reductive capacity of the cells further supports the amelioration of anthracycline cardiotoxicity.

In certain embodiments, doxorubicin elimination by drug efflux transporters in the cell membrane is considered when assessing the kinetics of doxorubicin release from the prodrug. Doxorubicin is a substrate of the efflux transporter P-glycoprotein (Pgp). In multidrug-resistant tumors overexpressing Pgp and/or other efflux transporters the rate of drug uptake in the tumor can be drastically reduced by the reverse rate of elimination, leading to effective drug resistance at tolerable doses. The prodrug form of doxorubicin has several advantages over doxorubicin alone. In one aspect, the amine functionality of doxorubicin plays a role in Pgp recognition and efflux, and modification of the amine has been reported to decrease Pgp efflux of doxorubicin. Since the prodrug form hides the amine group, it should have lower Pgp efflux than doxorubicin alone. In another aspect, drug efflux via membrane transporters occurs from within or immediately adjacent to the cell membrane, and doxorubicin release from the prodrug occurs in the cytosol, after partitioning out of the membrane. Therefore, once doxorubicin is released in the cytosol, it is likely to be far from the membrane and less likely to come in contact with Pgp.

pH-Dependent Cell Uptake

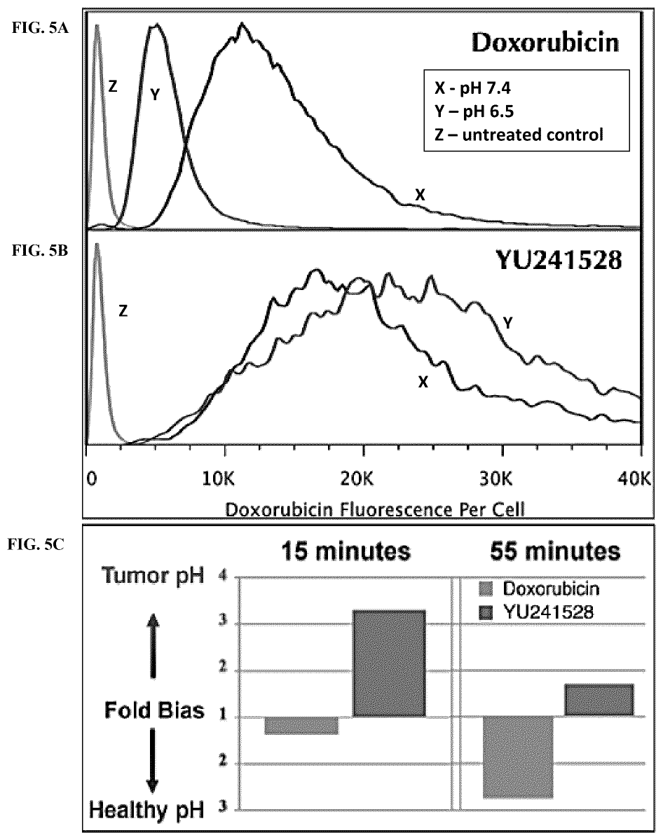

Doxorubicin and other weakly-ionic agents can exhibit pH-dependent cell uptake activity, due to their ionization differing in tissues with different pH environments. To test the pH-dependence of cell uptake for doxorubicin vs. the weakly acidic prodrug YU241528, tests were performed at pH 7.4 to represent healthy tissue extracellular pH and at pH 6.5 to represent tumor extracellular pH. HeLa cells treated in suspension for 15 or 60 minutes at the peak blood concentration of intravenous doxorubicin chemotherapy (5 μM) were washed, then analyzed by flow cytometry. In doxorubicin treated cells, the reported bias of the weakly-basic drug was readily apparent, as average doxorubicin fluorescence per cell was up to ˜3-fold higher in cells treated at pH 7.4 than at 6.5. In YU241528 treated cells, this bias was reversed, as average doxorubicin fluorescence per cell was up to ˜3-fold higher in cells treated at pH 6.5 than at 7.4 (FIG. 5).

One benefit of the present method is that it imparts a favorable pH-dependent selectivity to cell uptake of the prodrug. To assess the pH-dependence of prodrug versus free doxorubicin uptake in cells, cultured cells were treated in suspension. While the degree of uptake into cells in suspension likely differs from uptake in the more complex environment of a tumor, it allows assessment of relative cell uptake among drugs and between pH conditions.

The fold-biases for pH-dependent cell uptake were found to be equal and opposite between doxorubicin and YU241528, each resulting in ˜3-fold bias between pH conditions. This result agrees with the predicted fractions of non-ionized, and thus membrane permeable species predicted for each agent using the Henderson-Hasselbalch equation: the relative degree and orientation of the bias are equal and opposite for doxorubicin vs. the weakly-acidic prodrug. While in keeping with the orientation and relative scale of the biases, the absolute scale is not held, as the fraction of non-ionized prodrug is predicted to be far less for YU241528 than for doxorubicin. Without wishing to be limited by any theory, other properties such as Log P and molecular weight can also play a role in cell uptake and can be considered in addition to ionization in weakly-acidic drug and prodrug design.

pH-Dependent Cell Growth Inhibition

In order to evaluate pH-dependent activity, treatments are performed at atypical cell culture conditions. In order for different pH treatments to be comparable to one another without alteration of growth rate influences from the culture conditions, treatments are performed transiently in pH-controlled conditions, while at all other times during the assay, both before treatment and after the transient treatment period, cells are grown in normal culture conditions at pH 7.4. Several cell lines have been evaluated using this method and for each, cell seeding density and treatment duration is independently experimentally determined. Each experiment is normalized to sham and complete activity controls for each pH condition.

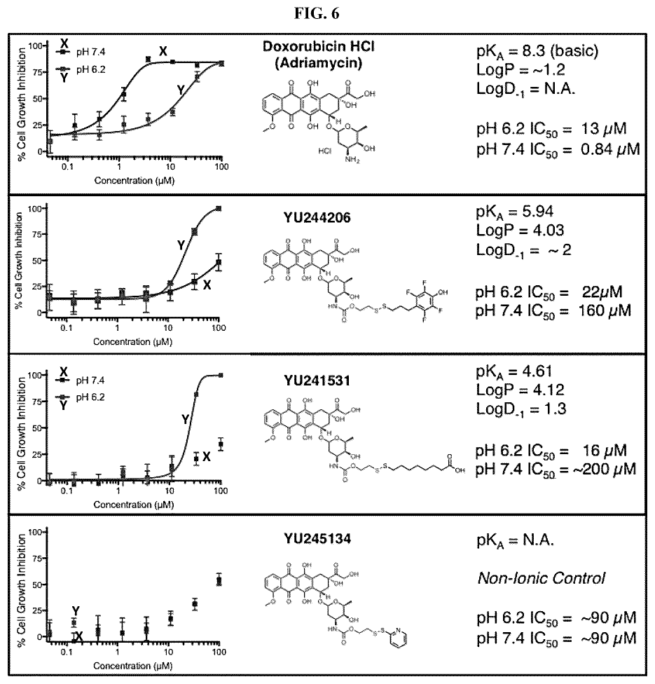

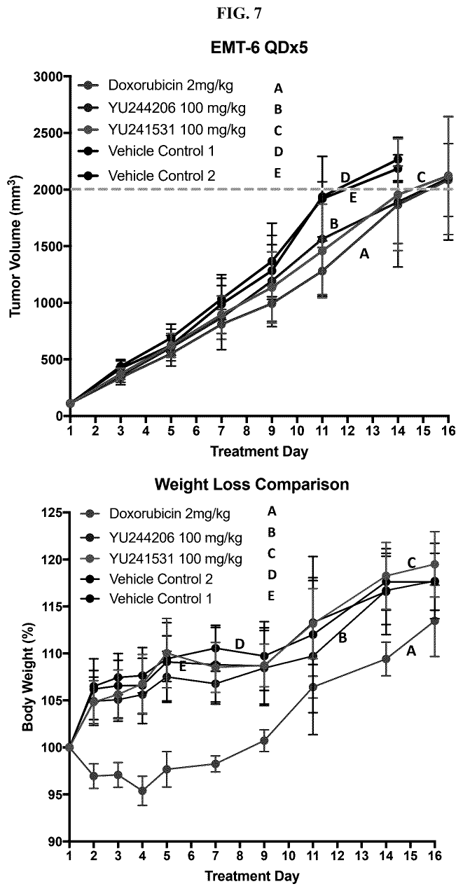

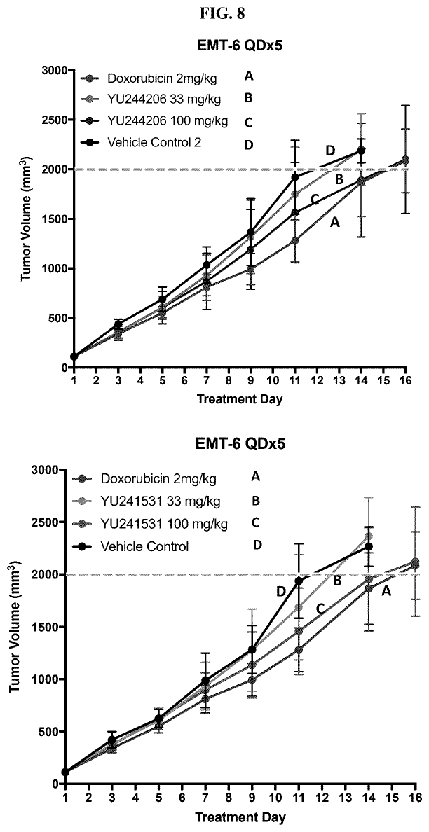

Human MDA-MB-231 breast cancer cells seeded at low density on tissue culture treated 384-well plates, allowed to adhere and grow in serum-supplemented growth medium for 24 hours, then treated under pH-controlled conditions for 6 hours, and finally washed and grown in fresh growth media for 72 hours were assessed for cell survival using the CellTiter-Glo assay system (Promega) (FIG. 6). The drug, doxorubicin HCl, is a weak base and so preferentially permeates cells at basic pH 7.4 compared with cells at acidic tumor cell surface pH 6.2, resulting in >15-fold lower IC₅₀ value in cells treated at pH 7.4 than at pH 6.2. Prodrugs bearing core acids, YU244206 and YU241531, are engineered to preferentially permeate cells at acidic tumor pH rather than pH 7.4, resulting in about 7 to 12-fold lower IC₅₀ values in cells treated at pH 6.2 than at pH 7.4. YU245134, which can be expected to be neutral in charge at both pH 6.2 and pH 7.4, showed no difference in cytotoxicity between the two treatment conditions.

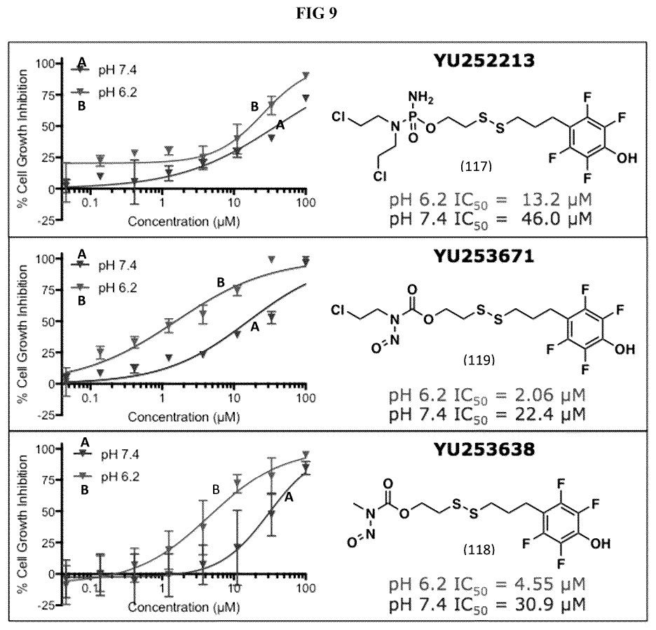

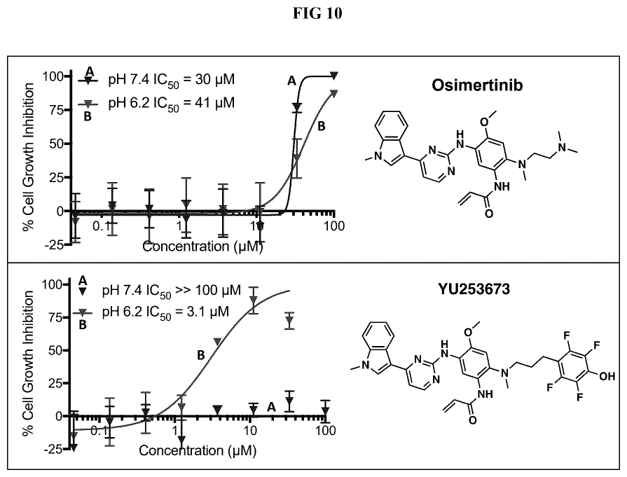

Similar results have been obtained from pH-dependent cell growth inhibition assays with other classes of Drug using the prodrug approach. In PEO1 cells, three prodrug compounds of the invention with DNA alkylating activity, YU252213, YU253671 and YU253638, exhibit 3.5 to 10-fold greater cell growth inhibition to cells treated at pH 6.2 than to cells treated at pH 7.4 (FIG. 9). Additionally, another compound of the invention, a weakly acidic derivative of an active core from the kinase inhibitor Osimertinib, YU253673, exhibits potent cell growth inhibition at pH 6.2 and no observed activity at pH 7.4, which reflects significantly more than 30-fold greater activity in tumor pH conditions compared with healthy pH conditions.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (1): 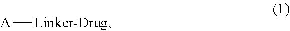
wherein:
A is an acidic group with pK_{A} ranging from about 4.5 to about 7.5,
Linker is a covalent bond or a chemical linker selected such that (1) is selected from the group consisting of: 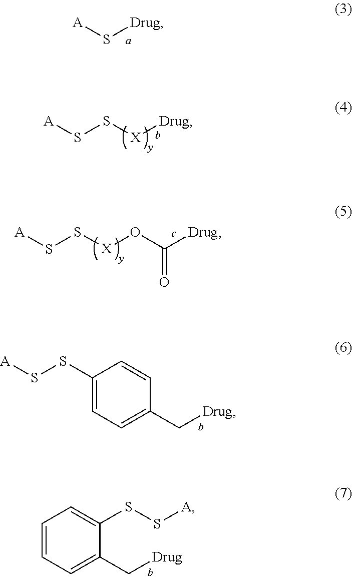
each occurrence of y is independently an integer ranging from 1 to 4;
each occurrence of X is independently selected from the group consisting of CH₂, CH(alkyl) and C(alkyl)₂;
bond a is formed between the sulfur and a substituent on Drug, wherein the substituent is a thiol;
bond b is formed between the carbon and a substituent on Drug, wherein the substituent is selected from the group consisting of hydroxyl, carboxyl, amine, amide, sulfate, sulfonamide, phosphate and phosphoramide;
bond c is formed between the carbonyl and a substituent on Drug, wherein the substituent is selected from the group consisting of primary amine, secondary amine, and hydroxyl; and
Drug is an anticancer drug;
or a salt, solvate, enantiomer, diastereoisomer, geometric isomer or tautomer thereof.

2. The compound of claim 1, wherein A is at least one of the following:
A is selected from the group consisting of: 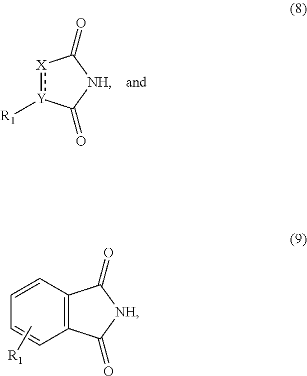
wherein [symbol] is a single or double bond;
wherein each instance of X is independently selected from the group consisting of C, N, S, and O;
wherein each instance of Y is independently selected from the group consisting of C and N; and
wherein R₁ comprises a covalent bond to Linker or Drug;
A is: 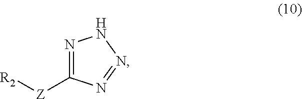
wherein Z is selected from the group consisting of N, C, and aryl, and
wherein R₂ comprises a covalent bond to Linker or Drug;
A is. 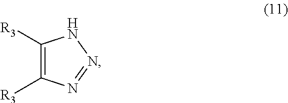
wherein each instance of R₃ is an independently selected electron withdrawing group, or one instance of R₃ is an electron withdrawing group and the other is H, or alkyl; and
wherein at least one instance of R₃ comprises a covalent bond to Linker or Drug either directly or by displacing a hydrogen on an electron withdrawing group, H or alkyl;
A is: 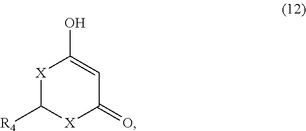
wherein each instance of X is independently selected from the group consisting of C, N, S, and O,
wherein R₄ comprises a covalent bond to Linker or Drug;
A is: 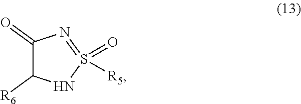
wherein R₆ is selected from the group consisting of an electron withdrawing group, an electron donating group, H, alkyl, and aryl,
wherein R₅ is selected from the group consisting of alkyl and aryl, and
wherein at least one instance of R₆ or R₇ comprises a covalent bond to linker or Drug.

3-6. (canceled)

7. The compound of claim 1, wherein y is 1 or 2.

8. The compound of claim 1, wherein A comprises a carboxylic acid.

9. The compound of claim 1, wherein Drug is a pharmaceutically active compound with anticancer, antineoplastic, antimitotic, proapoptotic, antimetastatic, antiangiogenic, cell growth inhibitory, cytostatic, antihormone, immunomodulatory, chemosensitization, or radiosensitization activity.

10. The compound of claim 1, wherein at least one of the following applies:
(a) Drug inhibits topoisomerase II activity;
(b) Drug inhibits topoisomerase I activity;
(c) Drug inhibits protein kinase activity;
(d) Drug has PARP inhibition activity;
(e) Drug inhibits estrogen receptor activity;
(f) Drug affects microtubule dynamics;
(g) Drug is a DNA-damaging agent.

11. The compound of claim 10, wherein at least one of the following applies:
(i) in (a) Drug is selected from the group consisting of: an anthracycline, an anthraquinone, podophyllotoxin, a quinoline-based compound, naphthalimide, elsamicin A, chartreusin, an acridine, salvicine, and derivatives thereof;
(ii) in (b) Drug is selected from the group consisting of: camptothecin, indenoisoquinoline and derivatives thereof;
(iii) in (c) Drug is an inhibitor of one or more protein kinases selected from the group consisting of: ErbB1, ErbB2, PDGFR, VEGFR, FGFR, ALK, c-Met CDK1, CDK2, CDK4, and CDK6;
(iv) in (d) the compound is selected from the group consisting of: 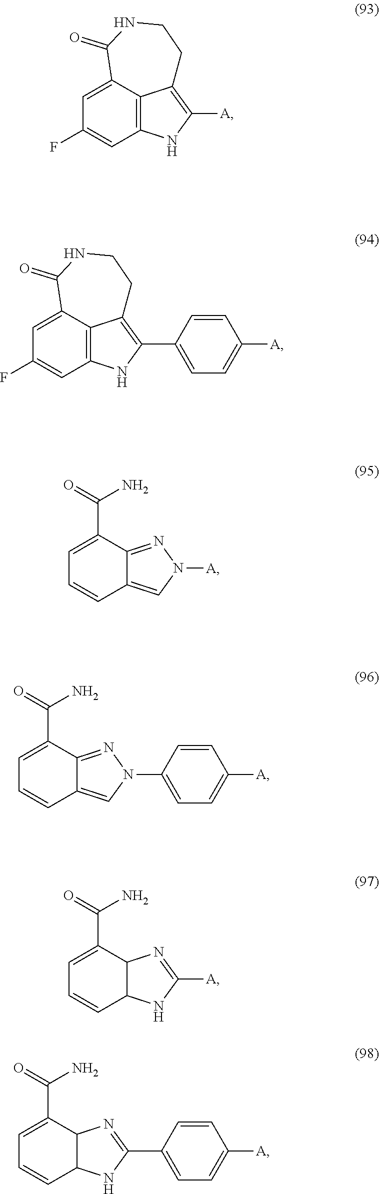 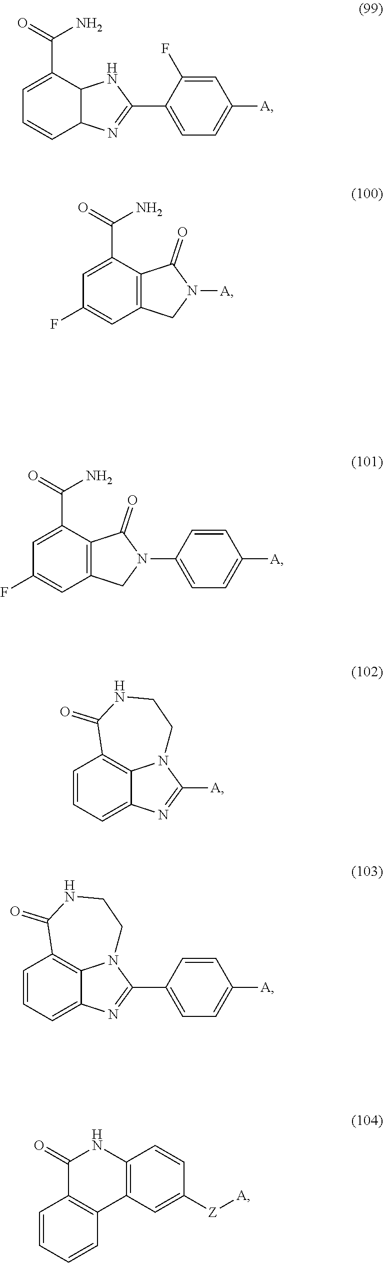 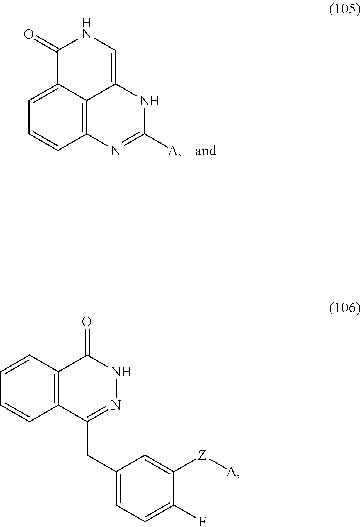
wherein Z may be present or absent and where present is independently selected from the group consisting of: O, S, NH, N(methyl), N(alkyl), and CH₂ and
wherein A is defined as above;
(v) in (e) the compound is selected from the group consisting of: 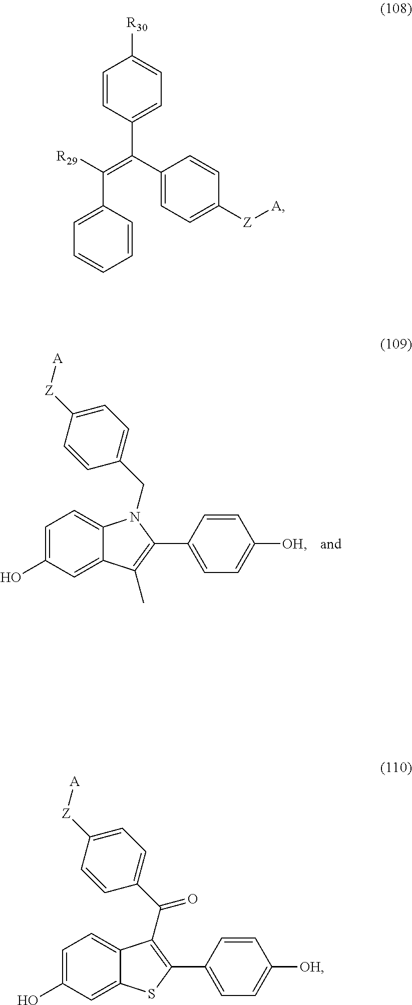
wherein each instance of R₂₉ is independently selected from the group consisting of: ethyl, Cl, and —CH₂—CH₂—Cl, and
wherein each instance of R₃₀ is independently selected from the group consisting of: H and OH
wherein Z may be present or absent and where present is independently selected from the group consisting of: O, S, NH, N(methyl), N(alkyl), and CH₂ and
wherein A is defined as above;
(vi) in (f) the compound is selected from the group consisting of: 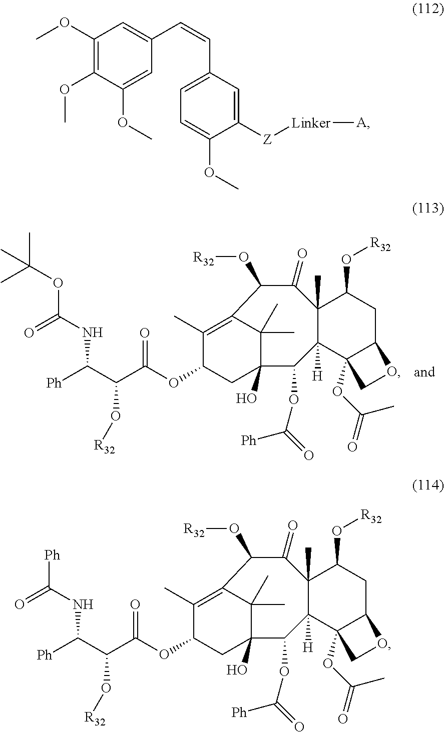
wherein each instance of Linker and A is as defined above,
wherein Z may be present or absent and where present is independently selected from the group consisting of: O, S, NH, N(methyl), and N(alkyl), and
wherein each instance of R₃₂, is independently selected from -Linker-A and H, provided that at least one instance of R₃₂ is -Linker-A;
(vii) in (g) the compound is selected from the group consisting of: 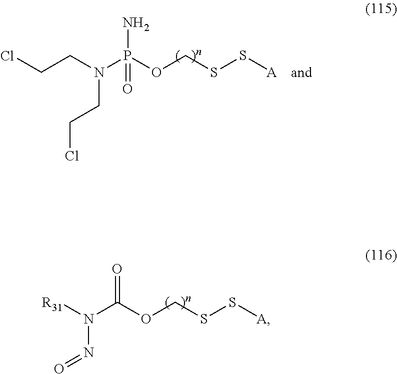
wherein each instance of n is an integer from 1 to 4,
wherein R₃₁ is selected from the group consisting of: methyl, alkyl, and —CH₂—CH₂—Cl, and
wherein each instance of A is defined as above.

12-13. (canceled)

14. The compound of claim 11, wherein at least one applies:
(1) in (ii) the compound is selected from the group consisting of: 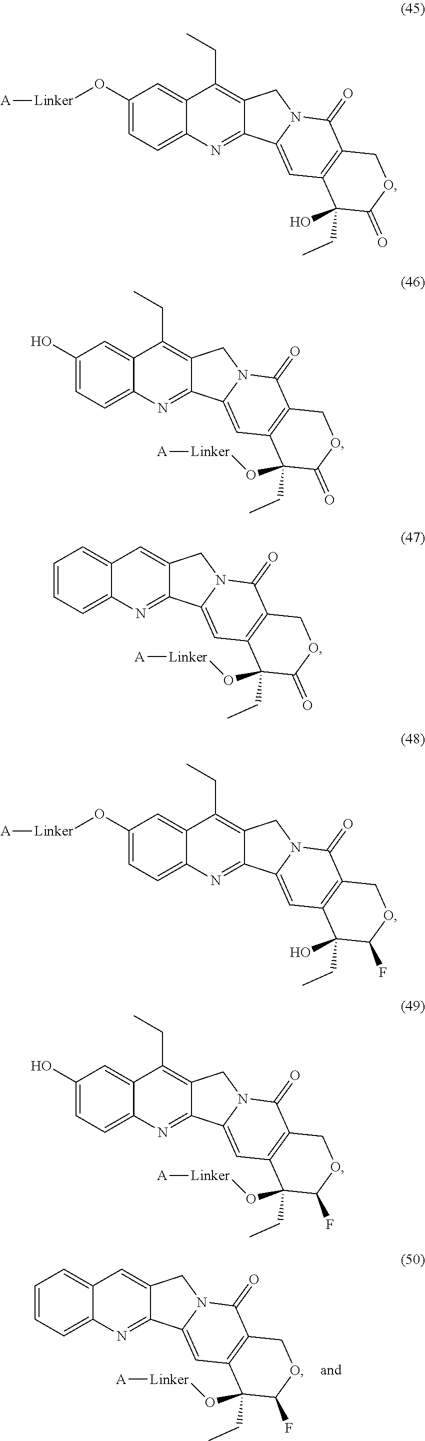 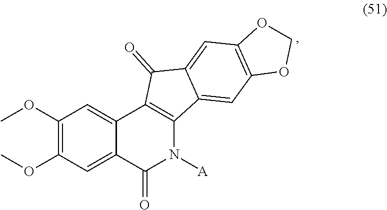
wherein each instance of Linker and A is defined as above,
(2) in (iii) the compound is selected from the group consisting of: 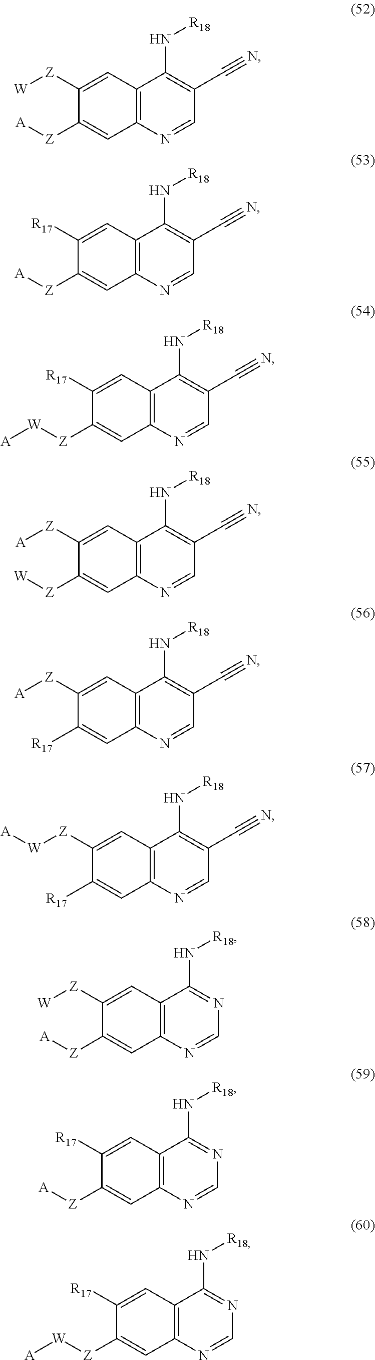 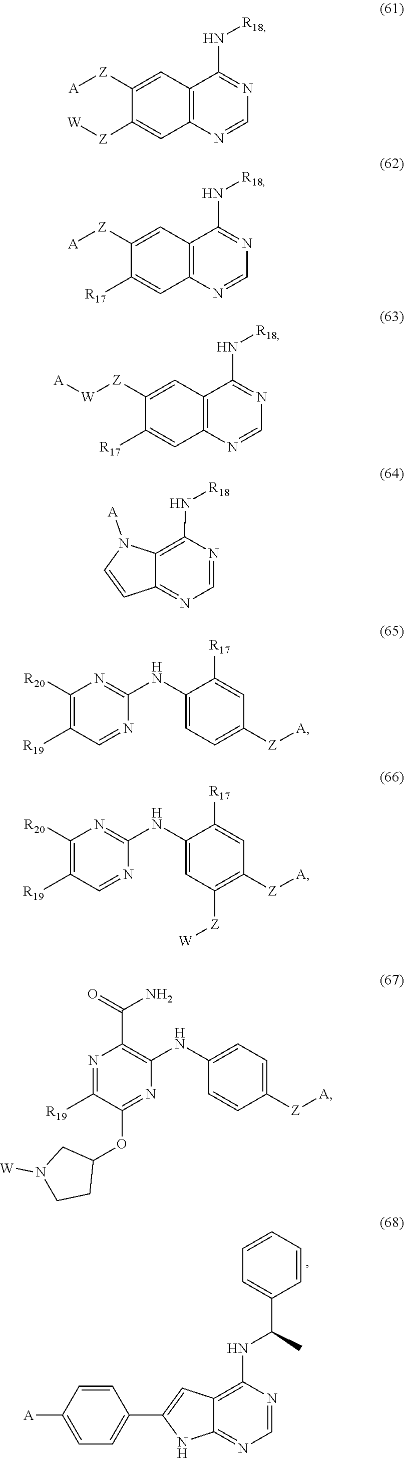 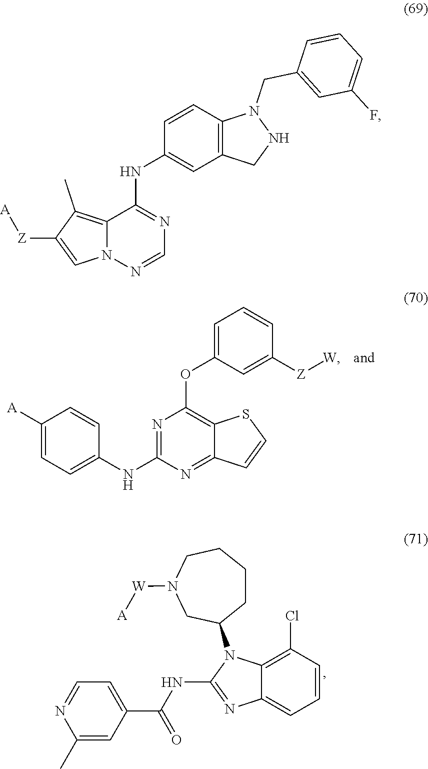
wherein each instance of R₁₇ is independently selected from the group consisting of: H, OH, —O—CH₃, —O—CH₂—CH₃, —O—CH₂—CH₂—O—CH₃, —O—CH₂—CH₂—OH, 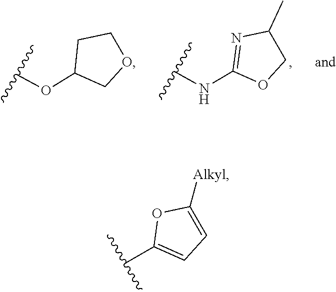
wherein each instance of R₁₈ is independently selected from the group consisting of: 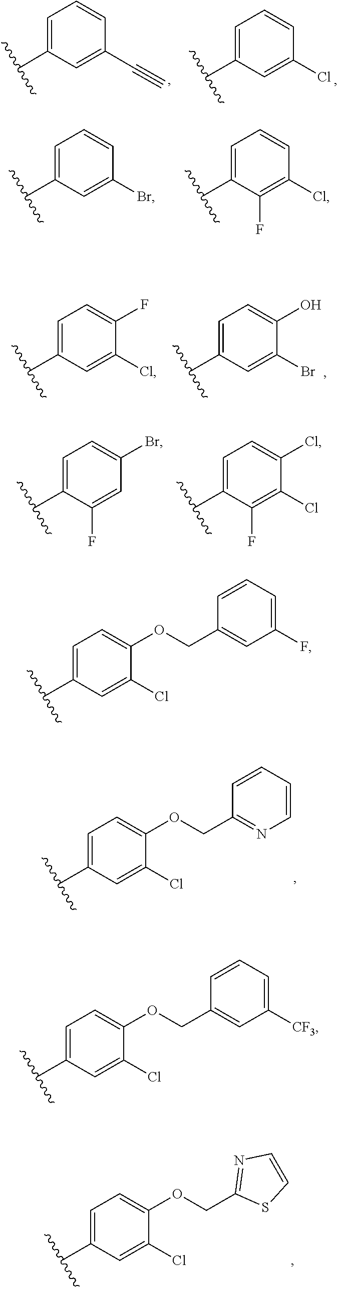 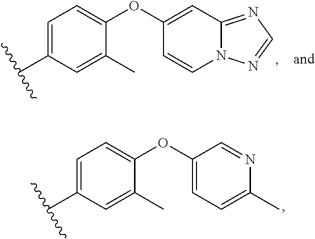 [SMILES: *c1ccc(Oc2ccn3ncnc3c2)c(C)c1 ; *c1ccc(Oc2ccc(C)nc2)c(C)c1]
wherein each instance of R₁₉ is independently selected from the group consisting of: H, F, Cl, Br, I, CF₃, CH₃, ethyl, and alkyl,
wherein each instance of R₂₀ is independently selected from the group consisting of: 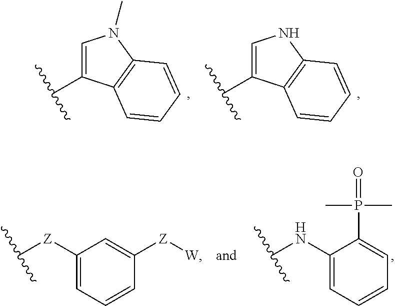
wherein each instance of A is defined as above,
wherein each instance of W is independently selected from the group consisting of: 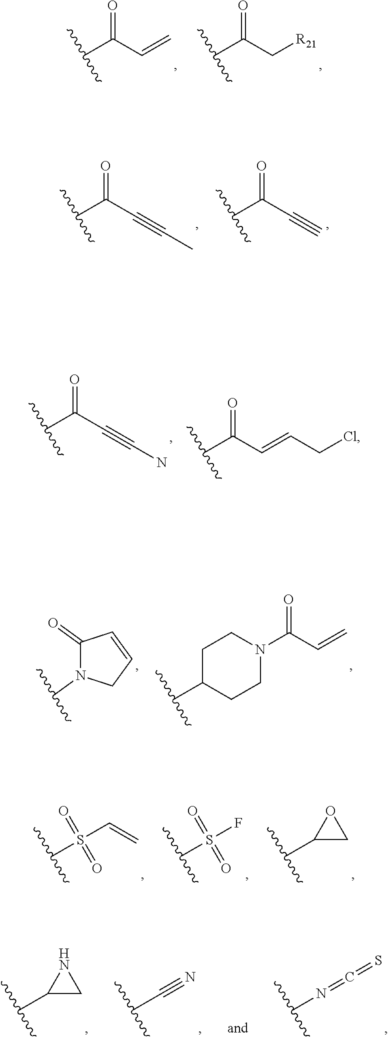
wherein each instance of R₂₁ is independently selected from the group consisting of: F, Cl, Br, I, and N₂;
wherein each instance of Z may be present or absent and where present is independently selected from the group consisting of: O, S, NH, N(methyl), N(alkyl), and CH₂,
wherein the covalent bond between A and W is made in place of a hydrogen on any CH₂ or CH₃ group in W;
(3) in (iii) the compound is selected from the group consisting of: 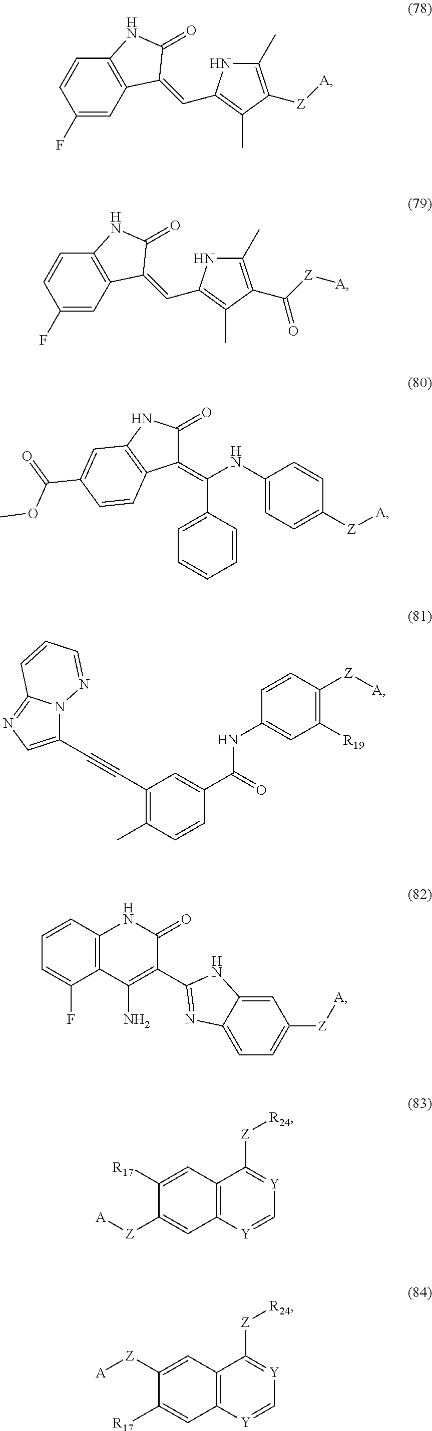 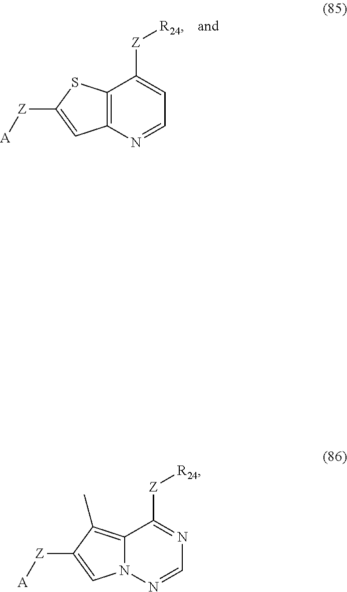
wherein each instance of R₂₄ is independently selected from the group consisting of: 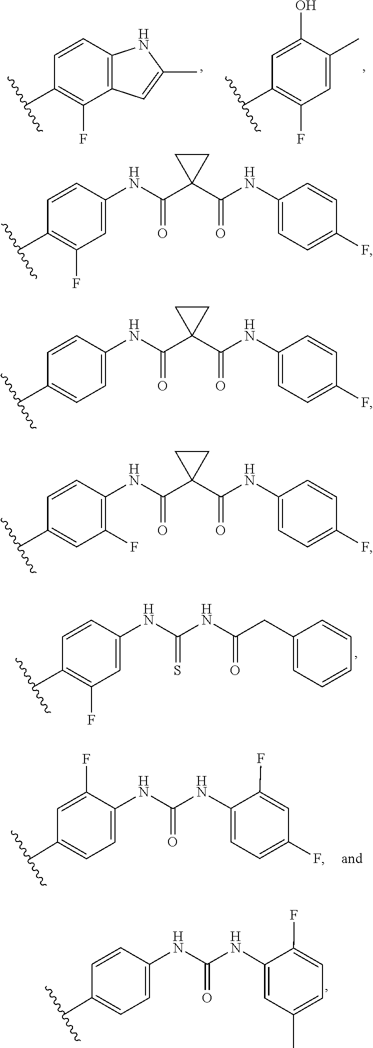
wherein each instance of R₁₇ is independently selected from the group consisting of: H, OH, —O—CH₃, —O—CH₂—CH₃, —O—CH₂—CH₂—O—CH₃, —O—CH₂—CH₂—OH, 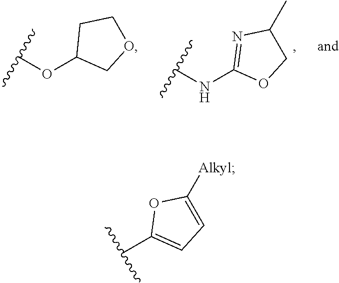
wherein each instance of R₁₉ is independently selected from the group consisting of: H, F, Cl, Br, I, CF₃, CH₃, ethyl, and alkyl,
wherein each instance of Y is independently selected from the group consisting of C and N;
wherein Z may be present or absent and where present is independently selected from the group consisting of O, S, NH, N(methyl), N(alkyl), and CH₂ and
wherein A is defined as above;
(4) in (iii) the compound is selected from the group consisting of: 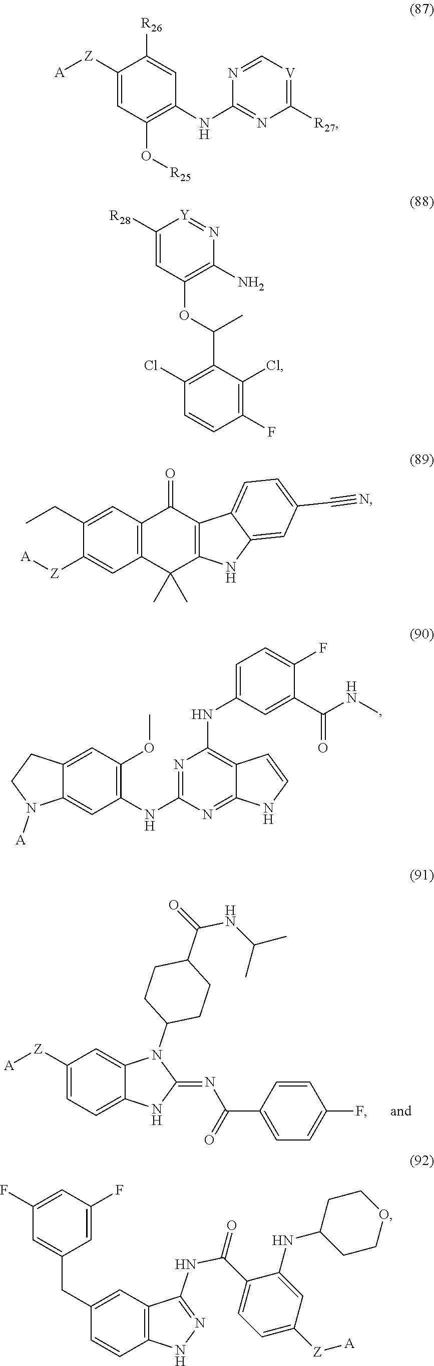
wherein each instance of R₂₅ is independently selected from the group consisting of: methyl and isopropyl,
wherein each instance of R₂₆ is independently selected from the group consisting of: H and methyl,
wherein each instance of R₂₇ is independently selected from the group consisting of: 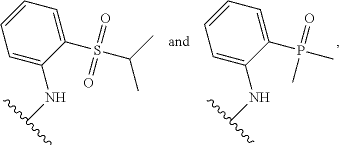 [SMILES: *Nc1ccccc1S(=O)(=O)C(C)C ; *Nc1ccccc1P(C)(C)=O]
wherein each instance of R₂₈ is independently selected from the group consisting of: 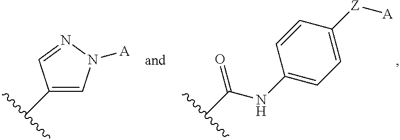
wherein each instance of V is independently selected from the group consisting of: N, CH and CCl;
wherein each instance of Y is independently selected from the group consisting of C and N;
wherein Z may be present or absent and where present is independently selected from the group consisting of: O, S, NH, N(methyl), N(alkyl), and CH₂ and
wherein A is defined as above.

15-27. (canceled)

28. A pharmaceutical composition comprising the compound of claim 1 and a pharmaceutically acceptable carrier.

29. The pharmaceutical composition of claim 28, further comprising at least one additional chemotherapeutic drug.

30. The pharmaceutical composition of claim 28, wherein the pharmaceutical composition is formulated for nasal, inhalational, topical, oral, buccal, rectal, pleural, peritoneal, vaginal, intramuscular, subcutaneous, transdermal, epidural, intratracheal, otic, intraocular, intrathecal, or intravenous administration.

31. A method for treating or ameliorating a cancer in a subject in need thereof, the method comprising administering to the subject a therapeutically effective amount of the compound of claim 1.

32. The method of claim 31, wherein the compound accumulates in a tumor cell to a greater degree than in a healthy cell in the body, and wherein the ratio of compound accumulation in the tumor cell with respect to the healthy cell is higher than for Drug alone.

33. The method of claim 31, wherein the cancer is at least one selected from the group consisting of melanoma, breast cancer, prostate cancer, ovarian cancer, uterine cancer, cervical cancer, skin cancer, pancreatic cancer, colorectal cancer, renal cancer, childhood solid tumors, soft-tissue sarcoma, non-hodgkins lymphoma, hepatocellular carcinoma, bladder cancer, testicular cancer, oropharyngeal cancer, head and neck cancer, and lung cancer.

34. The method of claim 31, further comprising procuring the compound of claim 1 for the subject.

35. The method of claim 1, further comprising administering to the subject additional cancer treatment.

36. The method of claim 35, wherein the additional cancer treatment is selected from the group consisting of radiation, surgical excision, immunotherapy, and antiproliferative chemotherapy.

37. A prepackaged pharmaceutical composition comprising the compound of claim 1 or the pharmaceutical composition comprising same and an instructional material for use thereof, wherein the instructional material comprises instructions for ameliorating or treating cancer in a subject.
